SEQUENCE LISTING (35 sequences)

SEQ ID NO: 1 - DNA, 2115 bp, Artificial Sequence
  Based on Glycine max sequence
atggggtctatgaatttgttaggtttttctctctctcctcacgaagaacacccttctagtcaagatcactctcaaacgacaccttctcgttttagcttcaaccctgatggatcaatctcaagcactgatgtagcaggaggctgctttgatctcacttctgactcaactcctcatttacttaaccttccttcttatggcatatacgaagcatttcacagaaacaatagtattaacaccactcaagattggaaggagaactacaacagccaaaatttgctattgggaacttcgtgcaataaacaaaacatgaaccaaaaccaacagcaacagccaaagcttgaaaacttcctcggtggacactcatttggcgaacatgagcaaacctacggtggtaactcagcctctacagattacatgtttcctgctcagccagtatcggctggtggtggtggtagtggtggtggcagtaacaataacaacaacagtaactccatagggttatccatgataaagacatggttgaggaaccaaccaccgaactcagaaaacatcaacaacaacaatgaaagtggtggcaatattagaagcagtgtgcagcaaactctatcactttccatgagtactggttcacaatcaagcacatcactgccccttctcactgctagtgtggataatggagagagttcttctgataacaaacaaccaaacacctcggctgcacttgattccacccaaaccggagccattgaaactgcacccagaaagtccattgacacttttggacagagaacttctatctaccgtggtgtaacaaggcataggtggacggggaggtacgaggctcacctgtgggataatagttgtagaagagagggacagactcgcaaaggaaggcaaggtggttatgataaagaagaaaaggcagctagagcctacgatttggcagcactaaaatactggggaacaaccacaacaacaaattttccaattagccactatgagaaagagttggaagaaatgaagcacatgactaggcaagagtacgttgcgtcattgagaaggaagagtagtgggttttctcgcggtgcatccatttatcgaggagtgacgagacaccaccaacatggaaggtggcaagcgaggattggaagagttgctggcaacaaggatctttacttgggaacttttagcacccaagaagaggcagcggaagcatatgatgtagcagcaatcaaattccgaggactaagtgctgttacaaactttgacatgagcagatatgacgtgaaaagcatacttgagagcaccactttgccaataggtggtgctgcaaagcgtttgaaggatatggagcaggttgaactgagtgtggataatggtcatagagcagatcaagtagatcatagtatcatcatgagttctcacctaactcaaggaatcaataacaactatgcaggagggggaacagcaactcatcataactggcacaatgctcatgcattccaccaacctcaaccttgcaccaccatgcactacccttatggacaaagaattaattggtgcaagcaagaacaacaagacaactctgatgcccctcactctttgtcttattcagatattcatcaacttcagctagggaacaatggaacacataacttctttcacacaaattcagggttgcaccctatgttgagcatggattctgcttccattgacaatagctcttcttctaactcggttgtttatgatggttatggaggtggtgggggctacaatgtgatgcctatgggaactactactgctgttgttgcaagtgatggtgatcaaaatccaagaagcaatcatggttttggtgataatgagataaaagcacttggttatgaaagtgtgtatggctctgcaactgattcttatcatgcacatgcaaggaacttgtattatcttactcaacagcaatcatcttctgttgatacagtgaaggctagtgcatatgatcaagggtctgcatgcaatacttgggttccaactgctattccaactcatgcacccagatcaactactagtatggctctctgccatggggctactacacccttctctttattgcatgaatag

SEQ ID NO: 2 - DNA, 891 bp, Artificial Sequence
  Based on Glycine max sequence
atgatggaacctcaacaacaacaacaacaagcacaagggagccaacaacaacaacaaaacgaggatggtggcagtggaaaaggggggtttctgagcaggcaaagtagtacacggtggactccaacaaacgaccagataagaatattgaaggaactttactacaacaatggaattagatccccgagtgcagagcagattcagaggatctctgctaggctgaggcagtacggtaagattgaaggcaagaatgtcttttattggttccagaaccacaaagctcgagaaaggcagaagaaaaggttcacttctgatcataatcataataatgtccccatgcaaagacccccaactaatccttctgctgcttggaaacctgatctagctgatcccattcacaccaccaagtattgtaacatctcttctactgcagggatctcttcggcatcatcttctgttgagatggttactgtgggacagatggggaattatgggtatggttctgtgcccatggagaaaagttttagggactgctcgatatcagctgggggtagcagtggccatgttggattaataaaccacaacttggggtgggttggtgtggacccatataattcctcaacctatgccaacttctttgacaaaataaggccaagtgatcaagaaacccttgaagaagaagcagagaacattggtgctactaagattgaaaccctccctttattccctatgcacggtgaggacatccatggctattgcaacctcaagtctaattcgtataactatgatggaaacggctggtatcatactgaagaagggttcaagaatgcttctcgtgcttccttggagctcagtctcaactcctacactcgcaggtctccagattatgcttaa

SEQ ID NO: 3 - DNA, 1134 bp, Artificial Sequence
  Based on Glycine max sequence
atggaaaacttttttgtgccatttttaaaaaaaaaccccaacccatcaatcaccactactggtggcaatggctcatcttcatcaaaccaaacaagccttgtacaaccaagcacatatcctcaaaatttcccttacaatactagtgtaaaacttaactttccagaacaaccttatttcattcctttgtatccctttccaacaggacaagttagcttttctaatcaaccctatggaatgccaaattcggaacttcaaggttcgagggcatgcatgaccaaagctacaagggagagatggagacaagtaagacaaaggagtaaaaattctactcttgtcgctcctaattcagttctagaaaggacaacaagagaacaatttgttcctaatggagggtcaaatgtgaggatcacagtcaaacaacacaatgcaaccaagttttttaacaccccaaacgggaagaagctagaagaaattttgacaaagaagttgaataatagtgatgttggcgtcctaggccgcattgtgctcccaaagagagaggctgaggataagcttccgacactgtggaagaaggaaggaatcaatattgtactaaaggatgtatattctgagattgaatggagcatcaaatacaagtactggactaataacaaaagcagaatgtatattcttgataatacaggggattttgttaaccattataaacttcaaacaggagatttcataaccctttacaaggacgagttgaaaaatctgtatgtgtcggctcgaaaggatcaagaaaatctagaagaatctaagtcctcgtcaaacacaggaatgtcacatgaaccagatgcatatttagcttacttgacgaaggaacttagccataaggggaaagcagaagctgccaacaaccttttgaacaatgttgaggaagaggcaccaaatcaagcaaatcaattacatcaattcatgccgatgaacaatattgttggggagggggcatcaaaccaagcaattcaagaagccgcaccagccgcacccgtcaatgttaatcaagaaaacaaagttgttgacgacgatgatgatgatatctatggtggccttgacaatattttcgaaattggaaatacttatcaaatttggtag

SEQ ID NO: 4 - DNA, 1038 bp, Artificial Sequence
  Based on Glycine max sequence
atgatgagtgcaagtgcaagaaataggtctcctttcacgcaaactcagtggcaagagcttgagcatcaagctcttgtttttaagtacatggttacaggaacacccatcccaccagatctcatctactctattaaaagaagtctagacacttcaatttcttcaaggctcttcccacatcatccaattgggtggggatgttttgaaatgggatttggcagaaaagtagacccagagccagggaggtgcagaagaacagatggcaagaaatggagatgctcaaaggaggcatatccagactccaagtactgtgaaagacacatgcacagaggcagaaaccgttcaagaaagcctgtggaagtttcttcagcaataagcaccgccacaaacacctcccaaacaatcccatcttcttatacccgaaacctttccttgaccaaccccaacatgacaccaccctcttccttccctttctctcctttgccctcttctatgcctattgagtcccaacccttttcccaatcctaccaaaactcttctctcaatcccttcttctactcccaatcaacctcctctagacccccagatgctgattttccaccccaagatgccaccacccaccagctattcatggactctgggtcttattcgcatgatgaaaagaattataggcatgttcatggaataagagaagatgtggatgagagagctttcttcccagaagcatcaggatcagctaggagctacactgaatcataccagcaactatcaatgagctcctacaagtcctattcaaactccaactttcagaacatcaatgatgccaccaccaacccaagacagcaagagcagcaacaacaacaacactgctttgttttggggacagacttcaaatcaacaagaccaactaaagagaaagaagctgagacagctacgggtcagagaccccttcaccgtttctttggggagtggccaccaaagaacacaacagattcatggctagatcttgcttccaactccagaatccaaaccgatgaatga

SEQ ID NO: 5 - DNA, 1128 bp, Artificial Sequence
  Based on Glycine max sequence
atggagggtagtagttgctctaatgacacttcttatttgttggcttttggagaaaacagtggtgggctatgcccaatgacgatgatgcctttggtaacttcccatcatgcaacaaatcctagtaatcctagtaataatactaataataatgaaaacacaaactgtctcttcattcccaactgcagtaacagttctggaactccttctatcatgctccacaacaacaacaacactgatgatgataacaacaaaaccagcactaacactgggttagggtactatttcatggagagtgaccaccatcaccgcaacaacaacaacaatggaagctcctcctcctcttcctcttctgctgtcaaggccaagatcatggctcatcctcactatcaccgtctcttggcagcttacgtcaattgtcagaaggttggagccccaccggaagtggtggcaaggttagaagaagcatgtgcttctgcagcgacaatggctggtgatgcagcagcagcagctggatcaagctgcataggtgaagatccagctttggatcagttcatggaggcttactgtgagatgctcaccaagtatgagcaagaactctccaaacccttaaaggaagccatgctcttccttcaaaggattgagtgccagttcaaaaatcttacaatttcttccaccgactttgcttgcaacgagggtgctgagaggaatggatcatctgaagaggatgttgatctacacaacatgatagatccccaggcagaggacagggaattaaagggtcagcttttgcgcaagtacagcggatacctgggcagtctgaagcaagaattcatgaagaagaggaagaaaggaaagctacctaaagaagcaaggcaacaattacttgaatggtggagcagacattacaaatggccttacccatccgagtcacagaagctggcccttgcagagtcgacaggtctggatcagaagcaaatcaacaactggtttattaatcaaaggaaacggcactggaagccttcagaggacatgcagtttgtggtgatggatccaagccatccacactattacatggataatgttctgggcaatccatttcccatggatctctcccatccaatgctctag

SEQ ID NO: 6 - DNA, 1407 bp, Artificial Sequence
  Based on Glycine max sequence
atgtccagcgccgccggagttcccgaccgcctcgcttcgcagccgcggggggctgccggcgcccctgccctcccgccgctcaagcgccaccttgccttcgtcacgaaaccgcccttcgccccgcccgatgagtaccacagcttctccagtgccgactcccgccgcgccgcggatgaagccgtcgtcgttagatctccgtacatgaagcggaagagtggaatgactgacagtgaaggggagtcacaagcacaaaagtggagtaacagcccaggatacactaatgttagtaatgtaacgaataatagtcccttcaaaactcctgtgtctgcaaaagggggaagggcacagaaggcaaaggcttccaaagaaggcagatcatgtcctccgacacccatgtcaaatgctggttccccttctcctcttactcctgctagcagctgtcgctatgacagttccttaggtctcttgacaaaaaagttcatcaatttggtcaaacatgcggaggatggtattcttgacctaaataaagcagcagaaactttggaggtgcaaaagaggaggatatatgacataactaatgttttggaaggcattggtctcattgaaaagaagctcaagaacagaatacattggaagggaattgaatcttctacgtctggtgaggtggatggtgatatctctgtgcttaaggcagaagttgagaaactttctttggaggagcagggattagatgatcaaataagggaaatgcaagaaaggctgaggaatttgagtgaaaatgaaaacaaccagaagtgccttttcgtgactgaagaagatattaagggcctgccttgcttccagaatgaaactttaatagcaattaaagctccgcatggaaccaccctggaagtccctgatcctgaggaagctgtagactatccgcagagaagatatagaatcattcttagaagcacaatgggccccattgatgtctaccttatcagtcaatttgaagagaaatttgaagaggttaatggtgctgagctccccatgatcccacttgcttccagttctggttccaatgagcaactaatgacggaaatggttcctgctgaatgcagcggaaaagaacttgaacctcaaactcagctctcttctcatgcattctctgatctaaatgcttcacaggagtttgctggtggcatgatgaagattgtcccttcagatgttgataatgatgcagattattggcttctatcagatgctgacgttagtataacagatatgtggagaacagattctactgttgattggaatggtatagacatgcttcatcctgattttggaatcatttcgaggcctcaaagtccatcatctgggcttgctgaagtgccatcaacaggagcaaactctattcagaagtga

SEQ ID NO: 7 - DNA, 729 bp, Artificial Sequence
  Based on Glycine max sequence
atgggtcgagggaaaatcgagatcaaaagaatcgacaatgctagcagcagacaagtcacgttctcgaagcggagaacagggttgttcaagaaggctcaggaactttccattctctgtgacgccgaggttgctgtcatagttttctccaacactggcaagctcttcgagttttccagttccggtatgaagcgaacactttcaagatacaacaaatgccttggttctacagatgctgctgtagcagaaattatgacacagaaggaagattctaagatggtggagattctaagagaggaaattgaaaagctagaaacaaagcaattacagttggtgggtaaggatctgacaggattgggtttaaaggaattgcaaaatttagagcagcaacttaatgaggggttattgtctgtcaaggcgagaaaggaggaattactcatggagcaactagagcaatctagagttcaggaacagcgggttatgttggagaatgaaactttgcgaagacagattgaggagcttcggtgtctgtttccacaatcagaaagcatggtcccattccaataccaacatactgaaagaaagaatacttttgtaaatactggcgccagatgtctcaacttggctaataactgtggaaatgagaaagggagttcagatacagcatttcatttggggttgcctgctggtgttcaagaggaaggcccccaagaaagaaaccttttcaaatga

SEQ ID NO: 8 - protein, 704 aa, Artificial Sequence
  Based on Glycine max sequence
MGSMNLLGFSLSPHEEHPSSQDHSQTTPSRFSFNPDGSISSTDVAGGCFDLTSDSTPHLLNLPSYGIYEAFHRNNSINTTQDWKENYNSQNLLLGTSCNKQNMNQNQQQQPKLENFLGGHSFGEHEQTYGGNSASTDYMFPAQPVSAGGGGSGGGSNNNNNSNSIGLSMIKTWLRNQPPNSENINNNNESGGNIRSSVQQTLSLSMSTGSQSSTSLPLLTASVDNGESSSDNKQPNTSAALDSTQTGAIETAPRKSIDTFGQRTSIYRGVTRHRWTGRYEAHLWDNSCRREGQTRKGRQGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELSVDNGHRADQVDHSIIMSSHLTQGINNNYAGGGTATHHNWHNAHAFHQPQPCTTMHYPYGQRINWCKQEQQDNSDAPHSLSYSDIHQLQLGNNGTHNFFHTNSGLHPMLSMDSASIDNSSSSNSVVYDGYGGGGGYNVMPMGTTTAVVASDGDQNPRSNHGFGDNEIKALGYESVYGSATDSYHAHARNLYYLTQQQSSSVDTVKASAYDQGSACNTWVPTAIPTHAPRSTTSMALCHGATTPFSLLHE

SEQ ID NO: 9 - protein, 296 aa, Artificial Sequence
  Based on Glycine max sequence
MMEPQQQQQQAQGSQQQQQNEDGGSGKGGFLSRQSSTRWTPTNDQIRILKELYYNNGIRSPSAEQIQRISARLRQYGKIEGKNVFYWFQNHKARERQKKRFTSDHNHNNVPMQRPPTNPSAAWKPDLADPIHTTKYCNISSTAGISSASSSVEMVTVGQMGNYGYGSVPMEKSFRDCSISAGGSSGHVGLINHNLGWVGVDPYNSSTYANFFDKIRPSDQETLEEEAENIGATKIETLPLFPMHGEDIHGYCNLKSNSYNYDGNGWYHTEEGFKNASRASLELSLNSYTRRSPDYA

SEQ ID NO: 10 - protein, 377 aa, Artificial Sequence
  Based on Glycine max sequence
MENFFVPFLKKNPNPSITTTGGNGSSSSNQTSLVQPSTYPQNFPYNTSVKLNFPEQPYFIPLYPFPTGQVSFSNQPYGMPNSELQGSRACMTKATRERWRQVRQRSKNSTLVAPNSVLERTTREQFVPNGGSNVRITVKQHNATKFFNTPNGKKLEEILTKKLNNSDVGVLGRIVLPKREAEDKLPTLWKKEGINIVLKDVYSEIEWSIKYKYWTNNKSRMYILDNTGDFVNHYKLQTGDFITLYKDELKNLYVSARKDQENLEESKSSSNTGMSHEPDAYLAYLTKELSHKGKAEAANNLLNNVEEEAPNQANQLHQFMPMNNIVGEGASNQAIQEAAPAAPVNVNQENKVVDDDDDDIYGGLDNIFEIGNTYQIW

SEQ ID NO: 11 - protein, 345 aa, Artificial Sequence
  Based on Glycine max sequence
MMSASARNRSPFTQTQWQELEHQALVFKYMVTGTPIPPDLIYSIKRSLDTSISSRLFPHHPIGWGCFEMGFGRKVDPEPGRCRRTDGKKWRCSKEAYPDSKYCERHMHRGRNRSRKPVEVSSAISTATNTSQTIPSSYTRNLSLTNPNMTPPSSFPFSPLPSSMPIESQPFSQSYQNSSLNPFFYSQSTSSRPPDADFPPQDATTHQLFMDSGSYSHDEKNYRHVHGIREDVDERAFFPEASGSARSYTESYQQLSMSSYKSYSNSNFQNINDATTNPRQQEQQQQQHCFVLGTDFKSTRPTKEKEAETATGQRPLHRFFGEWPPKNTTDSWLDLASNSRIQTDE

SEQ ID NO: 12 - protein, 375 aa, Artificial Sequence
  Based on Glycine max sequence
MEGSSCSNDTSYLLAFGENSGGLCPMTMMPLVTSHHATNPSNPSNNTNNNENTNCLFIPNCSNSSGTPSIMLHNNNNTDDDNNKTSTNTGLGYYFMESDHHHRNNNNNGSSSSSSSSAVKAKIMAHPHYHRLLAAYVNCQKVGAPPEVVARLEEACASAATMAGDAAAAAGSSCIGEDPALDQFMEAYCEMLTKYEQELSKPLKEAMLFLQRIECQFKNLTISSTDFACNEGAERNGSSEEDVDLHNMIDPQAEDRELKGQLLRKYSGYLGSLKQEFMKKRKKGKLPKEARQQLLEWWSRHYKWPYPSESQKLALAESTGLDQKQINNWFINQRKRHWKPSEDMQFVVMDPSHPHYYMDNVLGNPFPMDLSHPML

SEQ ID NO: 13 - protein, 468 aa, Artificial Sequence
  Based on Glycine max sequence
MSSAAGVPDRLASQPRGAAGAPALPPLKRHLAFVTKPPFAPPDEYHSFSSADSRRAADEAVVVRSPYMKRKSGMTDSEGESQAQKWSNSPGYTNVSNVTNNSPFKTPVSAKGGRAQKAKASKEGRSCPPTPMSNAGSPSPLTPASSCRYDSSLGLLTKKFINLVKHAEDGILDLNKAAETLEVQKRRIYDITNVLEGIGLIEKKLKNRIHWKGIESSTSGEVDGDISVLKAEVEKLSLEEQGLDDQIREMQERLRNLSENENNQKCLFVTEEDIKGLPCFQNETLIAIKAPHGTTLEVPDPEEAVDYPQRRYRIILRSTMGPIDVYLISQFEEKFEEVNGAELPMIPLASSSGSNEQLMTEMVPAECSGKELEPQTQLSSHAFSDLNASQEFAGGMMKIVPSDVDNDADYWLLSDADVSITDMWRTDSTVDWNGIDMLHPDFGIISRPQSPSSGLAEVPSTGANSIQK

SEQ ID NO: 14 - protein, 242 aa, Artificial Sequence
  Based on Glycine max sequence
MGRGKIEIKRIDNASSRQVTFSKRRTGLFKKAQELSILCDAEVAVIVFSNTGKLFEFSSSGMKRTLSRYNKCLGSTDAAVAEIMTQKEDSKMVEILREEIEKLETKQLQLVGKDLTGLGLKELQNLEQQLNEGLLSVKARKEELLMEQLEQSRVQEQRVMLENETLRRQIEELRCLFPQSESMVPFQYQHTERKNTFVNTGARCLNLANNCGNEKGSSDTAFHLGLPAGVQEEGPQERNLFK

SEQ ID NO: 15 - DNA, 1000 bp, Artificial Sequence
  Based on Glycine max sequence
aatattattaatatactcttaatatattggttaatgaaataaaattaattattgatttcttaattacttattcttgaagtatacagattcataaaatctcttcttacaatggacacaaaaactaagcatcttttcgtttacaatgtgtcattagcatcttcttaatcttcttaattaatgaatctctattagcgattacaatgtgtcattaacatcttattcgatagtactattaattgagattcctctcattcaaccacttttataaaaaaataaagttttaacaaaaaagaaaatcatagttcataatatctaactttatactttatgaaaaaaaagtaatgtatcacatatcacatcagaatttattttccatgaaacatgaaggcagtgatgcatcaatcagcacattagtgattttgtgtcacaagtcacaactgttcagaaaaagctcttagagtgaatcgtaacaccgtatcacaagggcgcattatatttttcaataccgcgagcaactagtagtactagtgtgtttggactaccacattaattacgaaatggtccccgtgtgtggatcttttcattagcccttgaagtaattttttttttctgattcaaagatttcaagtgccctagaatgtataagacgcgtcccatttctattgtgtgcgcgtgtgtggtgtgtacgtgcatatcagccagaagaaagagaaaataactcaaaatatagtaacttaaagtatactataaatgttctctcatctctatgctataaatgtttttttttcaattttttgagctcttcaagaatttgacccttctcctcctcctccttcttcttttctttcaaacctcctcatataaactagtactatatgcttcttcttcttcttctccttcatgcacaaactgctattttcaccctttatatatctatctactcctgaagattagattaccttgagggctttgtgctctctgtgtaatattcttcaatatc

SEQ ID NO: 16 - DNA, 1000 bp, Artificial Sequence
  Based on Glycine max sequence
tgaaatgcctatagaatatgcggaccaatgcacaacacaaaaaataaatagccctgatggaaagggaaattcgatctaaatctacatctcatcttttaataagtgtatgtacggaaagaggagagatataaaaaaaataaaataatagatataataaattacttatttgatgaaaaataaaagttaaaatataaaaagagaattgaagtaaaagtgagatggaaaaaaaaaatggatgtatcaccaattgaccataataactctatatgcttcatgcattggttgggacccatgaaatgcacaataagttcacaaatacatttttaccctccaattcatcaggtaagtacagaatatatatcttggtagcttgctgattcgacttaataattatagagtaagaatttaaaaaaaaaatgtatgtgtgtgtataggggccatgtctgatatctccatcaaaagaagaacctattgaactcccaaatcacaacccgcatcattccattgccattcattcattcattcagaaaatctactcttttttttttctttccttccatccaatatatcatttcatgcctcatttttctaccttttcccactgtctctgtgtgcaaatactttatttcacacatacctggtcatgccttttcgtccaagtaattcctgatagtaccctcactttctaagctctcttttgtcccttccctttttatgaacaccactctgtcaccctcagtccttctctctcagatatttatttatgattttctctctttatcactccatgtactatatgtgcctgtgcctcatctatcatctatcatctatcatctatcatcacctattataagtttataacccccctcaccctttcctccccttcataattcatgcagtagtaatctctcttctcacctatataccctctaatattctaattctctctcttgatccaacaaacaaacactaccattttgtttgttctgagtagtgatcc

SEQ ID NO: 17 - DNA, 1000 bp, Artificial Sequence
  Based on Glycine max sequence
acacttatttttttcttcaatcacattcacgtatattattatatattctataatatttgtatttattcaattcaattatttattatttttttatatttattaacatatataaatgataattaaaaacatattcaattcaataataatattatatattattatacactaattaataagtcacatttatgtgtatataccaattgactgtaatattatcttttagattttaataagtcacacacgcatgcataaagacgattttaatcagacatattcatgtatattatcatatactaattaataaatacctatgtgatattttcattgattgcttatgaaactctcaaccccacacatgaagccaaaaccatggccaaaccaaaaccccagccattttcacacctctatcttcccatagtcacttcctatattattatcctctcttcgtaactgcaattcatgttcctctaggcatcttacaaacacatggggcacacacctttctttggctttatgcaacacatgaagacaatgtccatcttgcataccatttataagtcagcaagtctcaactttatgataccataacgctcactttcactgcaatgacatttcatcttctcttgttttttctgcttcatccatctcaacactctcaatttttttttatattttgaacttgcaatttatgtgtttttgttcagtgcatttgattacaactcagatgagtattccaatgtcacaacgttccctccacttgttacccacttcaacatcttccttcctctctcttgtttccttttccttccttttctttattctcgttcacaatccttgcatttatttttgtcatacttttttttttatatttttgtttgcttaattggcactaccactgcacctaaacaacttcttataagagcctcatacacacacacactctctcaattcactcaacactcaaaacaaaaaccttgaagcctgttaatttctcaccaaa

SEQ ID NO: 18 - DNA, 1000 bp, Artificial Sequence
  Based on Glycine max sequence
attatcattgagttaaaactctaactcaagcatgaaaaaatacattaaagttttgtgtttttcaattaccataaagtttgatgaatattggttttgacgttttgtggttatggaaatgattaaggagaaaacatgtaaagggttatgatggcctattgacaagacggtggccaatagagagttaaaggccaaattgactgtaacccaaattccactgatgaaagtgagatgcttgggtttggggggtgaaatgaaaaaaggagaaaggagaaagcatcaatccgtggccaaaaaaagcaggattcagctctagccttggcctccaaatctatcaatgagataacgccacgcatgcttcaagccaaaaaagattaaaaatgacacgtacgagactttctcttattcaaaaagttactacaattgcaaagagagattgataatttgatatactaatggccactattgctcagcagcttacacttcacataaccggatggcatggcactgttttccatgaagtgatgtggagacagcaaaaccaaaggtgcatggactaacatgcatttgaatttaatttttcttcttttcctttgtacatttgtttatggatttctgtaaagatgttagagacaagggcagcaacaaaggcagctgcagagaaaaaacagaagcaacagaggtgcagtcattataaagagcagactcactcactcacccatcatccagcacattagagaaatagagaggaggtggcagcaaagccagaaagcatcatcagactctcagacccattagtattatccgtgcacaggagaagaatctctacccttgaaaaatatatataaaaataaaataataatgaccctccaaagtccaaattactatcaccccatctagagaatttatttcactctttcaaatcttatatcttcttgttcttcacttccccactattttagagagagacacacacactcttccttccttttgttgtctcaaa

SEQ ID NO: 19 - DNA, 2000 bp, Artificial Sequence
  Based on Glycine max sequence
tgcacatgcaatttaattgtgatatcattattatcactcatatgaagctattgctagctcaaatagtagtattaatttattattagaactttcaagaactaagcgtacgttcaagtatcaatcaatcaacacaatttgctcgataatgataacatactcgtatacacctagctcacataagttacggtattaaacatttataatctgacacaatttaatatcattatcgagctgttatcatatttaagttaaggatttctttaattagtatttttaagatattaattaaaaaaaataaaaaaatatttattgtgtaaatcaagataaaaaattatatctctcaataaaaatatttttactttaaatttcttaactaatattcttaaaacacttattaatatttatttttaggttaaaagtaaaagtatttataagaaacagtaatagaaaaattaaatatataatagttaataattaataatttgttattaaaatgacatcataccttactggctcttagaaaatcaattcttatagttgtagtactttttataacagaaaacattatatttcaaattgaagtgtactcaagaaaaaaaatgaaatgaagagtataaccgggagagggggacaatgggaagcgacaatgtgtacgtaacctgatggaggtgctttcactacggtattttacgggaagtgatgctacgctaggcctttattaattattatattagggacgagggatatcatatgggatatagagatgaactatggtgctggaaatagatcgagaaaaaaggggttgctgagaggaagagacattcggactgtcccacaaactttaccagctttatttactcacctgcagacgcgctttttccatggttaattatactgtatcgtattaaattagatcatactagtatactatatactaccataggaagagagagaagtaagcatcatcatatagtaaatattcatgtttagactttagtattaatagtaactaacgctaatgttaaaacactaaatacatctattttggagctaacaagaagaacaaattaggtttgataaattaaatccctaatgttctgttaaatgttggtacttgtttgtgggactagagaattttttaatcactgtggtgagaagatcgaggacaaatagggtgagaatattaaatgagtggagggattgccatcaaagtgtagagagagagagaaggaagggttgattttgattccgtgccccataaacataaacataaacataaaccatctcatctttctccattgatggccagtagtgggtaacttgtttttcttcctcgatttgatcgttccttctctctctctattgtgttttgttttatgccaggaatggcagcgtatcagtggcagtgcaggaaaagagagggagagttttcattgggaaggtaaaagcttttgtttgtagcagtgaaacctcgcccccttctcttcatcgctactagtagtaactcatcgttttctcggtgtgccccgcgtgcgctctgctgtgtcttctcactcacaccagaggtgtaaccgtgtaaccactagaatcatttattcattaatgctggcaacagtggcatggaaagaaagattaatttttccaaaggaaagaaaaaccctctgcaggctttgccagataagccaagtgggaaaaccaaaccctctattagtacttacttcatgtaactgactatagccaccactatcactatttaggattttctgtaaaaagcctgatactcttttaccataaaacccgggagagccctggaagacaaacatcttcattcagacttcataaaataaaatagagaagtgtttttttgtttttttggtttgttgtaattaaggctagctagtgagtgtgttctacaactgtagtgagctacagaaggtggtggtagtagtaggcaaaaaggataagacagtgagtgtgtatgttgttgacaagcaaaagcc

SEQ ID NO: 20 - DNA, 1000 bp, Artificial Sequence
  Based on Glycine max sequence
aattagtcttattgaatacttataatttaataagttaacttcccaattttagattatcaaatttctagtttcacggaacataacctattttcaaaaataatttaacataacacttaatttggtatactaacacacatgtacattcattaaaaaatagactaagtaattgataatatattacaaaattaaaacatataaactaattataaattattaaatatgattttatacctgtgctagacatgtggtatcacgctagtaattaataatatattaaaaattaaaataatataacaagttactactataaattataaaatataaatgtaatatcaatataagccacaagagttaaacttgtccatatgtataacttttaagtagttagaaaacttgttaaagatataaaatttattgacgatataaattttgtttacaccagtatcaatgcatatcaattaaatcctttttctattaattttaacatatacatcacattaatcacactaatgaaggtaagcaaagaatttaacaagtttttttttttttaaaatctaatataaactaaaaagtaaggcagcgaaaaaggaaataagataatttcatgataataatctaaaaatacaataaccccgtaccaaaaaaacatgtgtaattacaggaacacttaaaatttcttcttttattattattattttttttttcgcgcatgcagttccctccacatctatccgaaaccaaattccctccttccctcgttttctgctctcgcctcctctacgttccataacgccctctctctctctctctctctctctctcttttttttttttttttccaaacccttttcccctccctctcactttctctctctaaaccccactctttctctctctaaaaccctacactgtactctccttccttcggatccttctcccgtttccctccaatttccccccaattccgctggccccacctccgccccttttcccgcttcctc

SEQ ID NO: 21 - DNA, 270 bp, Artificial Sequence
  Based on Glycine max sequence
tctaaatgcccagagaacacaacacggagccatgcaaagttgccgtttccagcaaacctctctggttatttgaggtaaaacgctttgcagtctcgcaaatcgcaacaaccccttcgtcttctcagtaaaaggggtcttacttacttagtgtcttcgttcgtatcttcaaccctgaattcgcttctcctcccaaagcaccaccaccacctctaattaattcctcgttcagttgggcatgtttgcgcatttctgagagagcgagaaaataaa

SEQ ID NO: 22 - DNA, 327 bp, Artificial Sequence
  Based on herpes simplex virus sequence
ggagggtccggaggtgacgctttggatgatttcgatctcgatatgctcggctccgacgcccttgatgacttcgacctggatatgcttggaagcgacgctctcgatgacttcgatcttgacatgcttggtagtgatgccctggacgactttgacttggatatgctcgctcgggggtccgacgctttggatgacttcgatctggacatgctgggctcagacgcacttgacgacttcgacctcgacatgctgggatcagacgccctcgatgattttgatcttgacatgcttggaagtgacgcgttggacgattttgatctcgatatgctt

SEQ ID NO: 23 - DNA, 1020 bp, Artificial Sequence
  Based on Xanthomonas campestris sequence
ggagggtccggaggtctgttggaccctggtacgcctatggatgcggatttggtcgcgtctagtaccgttgtgtgggagcaagacgcggacccgtttgcaggaacagcagatgattttcccgcgtttaatgaagaagagttggcctggttgatggaacttcttcctcagggaggttcgggaggacttcttgaccccggcactccgatggatgccgacctcgtcgcatcctctactgtcgtttgggaacaggatgcagacccgttcgcaggcaccgcagatgatttccctgcctttaacgaagaggaactcgcttggctgatggaattgcttccgcaagcgagagggggttcaggcgggttgctcgatccgggtacaccgatggacgccgacttggttgcatcgtcaacagtcgtctgggaacaggacgcggacccctttgcgggcacagcggacgacttcccggcttttaatgaggaggaactcgcatggcttatggagcttttgccacagggtggttcaggtggtctacttgatcctgggactcctatggacgccgacttggtagctagctcaacagttgtttgggagcaagacgctgaccctttcgccggcactgcagacgattttcccgctttcaatgaagaagagctcgcctggctcatggagcttctgccccaggctagaggaggctcaggtggattgctggatccaggcaccccaatggacgcagatctcgtcgctagtagcactgtagtgtgggaacaggatgcagatccctttgctggcactgccgacgacttccccgcattcaacgaggaggaactggcttggcttatggaactcctccctcagggggggtccggcggcttgctggatcccggcactcccatggacgcagacctggttgcttctagtaccgtcgtctgggagcaagacgccgatccattcgcaggtaccgccgatgattttcctgcctttaatgaagaagagttggcatggttgatggagctccttcctcaa

SEQ ID NO: 24 - DNA, 1368 bp, Artificial Sequence
  Based on herpes simplex virus sequence and Xanthomonas campestris sequence
ggagggtccggaggtctgttggaccctggtacgcctatggatgcggatttggtcgcgtctagtaccgttgtgtgggagcaagacgcggacccgtttgcaggaacagcagatgattttcccgcgtttaatgaagaagagttggcctggttgatggaacttcttcctcagggaggttcgggaggacttcttgaccccggcactccgatggatgccgacctcgtcgcatcctctactgtcgtttgggaacaggatgcagacccgttcgcaggcaccgcagatgatttccctgcctttaacgaagaggaactcgcttggctgatggaattgcttccgcaagcgagagggggttcaggcgggttgctcgatccgggtacaccgatggacgccgacttggttgcatcgtcaacagtcgtctgggaacaggacgcggacccctttgcgggcacagcggacgacttcccggcttttaatgaggaggaactcgcatggcttatggagcttttgccacagggtggttcaggtggtctacttgatcctgggactcctatggacgccgacttggtagctagctcaacagttgtttgggagcaagacgctgaccctttcgccggcactgcagacgattttcccgctttcaatgaagaagagctcgcctggctcatggagcttctgccccaggctagaggaggctcaggtggattgctggatccaggcaccccaatggacgcagatctcgtcgctagtagcactgtagtgtgggaacaggatgcagatccctttgctggcactgccgacgacttccccgcattcaacgaggaggaactggcttggcttatggaactcctccctcagggggggtccggcggcttgctggatcccggcactcccatggacgcagacctggttgcttctagtaccgtcgtctgggagcaagacgccgatccattcgcaggtaccgccgatgattttcctgcctttaatgaagaagagttggcatggttgatggagctccttcctcaagcacgcggggggtctggtggtggtggatctggcggtgacgctttggatgatttcgatctcgatatgctcggctccgacgcccttgatgacttcgacctggatatgcttggaagcgacgctctcgatgacttcgatcttgacatgcttggtagtgatgccctggacgactttgacttggatatgctcgctcgggggtccgacgctttggatgacttcgatctggacatgctgggctcagacgcacttgacgacttcgacctcgacatgctgggatcagacgccctcgatgattttgatcttgacatgcttggaagtgacgcgttggacgattttgatctcgatatgctt

SEQ ID NO: 25 - protein, 106 aa, Artificial Sequence
  Based on herpes simplex virus sequence
GSDALDDFDLDMLGSDALDDFDLDMLGSDALDDFDLDMLGSDALDDFDLDMLARGSDALDDFDLDMLGSDALDDFDLDMLGSDALDDFDLDMLGSDALDDFDLDML

SEQ ID NO: 26 - protein, 340 aa, Artificial Sequence
  Based on Xanthomonas campestris sequence
GGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQ

SEQ ID NO: 27 - protein, 456 aa, Artificial Sequence
  Based on herpes simplex virus sequence and Xanthomonas campestris sequence
GGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGGGSGGDALDDFDLDMLGSDALDDFDLDMLGSDALDDFDLDMLGSDALDDFDLDMLARGSDALDDFDLDMLGSDALDDFDLDMLGSDALDDFDLDMLGSDALDDFDLDML

SEQ ID NO: 28 - DNA, 7092 bp, Artificial Sequence
  Synthetic construct based on Agrobacterium tumefaciens sequence, Aequorea victoria sequence, Brassica napus sequence, Xanthomonas sequence, and Flavobacterium okeanokoites sequence
cgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttcgaatacatctagatccaagtacatggcaaataatgattttattttgactgatagtgacctgttcgttgcaacaaattgatgagcaatgcttttttataatgccaactttgtacaaaaaagcaggcttaggtacctcgcgaatgcatctagatccaatgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgattacccatacgatgttccagattacgctatggctcctaagaagaagagaaaggttataacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccgcggttcccgggagatcttggagggggcggtagcggcggtggcgggagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcattggatcggatcccgggcccgtcgactgcagaggcctgcatgcaacaactttgtatacaaaagttgaacgagaaacgtaaaatgatataaatatcaatatattaaattagattttgcataaaaaacagactacataatactgtaaaacacaacatatccagtcactatgtcgattgtcttcatcggatcccatcccctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggc

SEQ ID NO: 29 - DNA, 4232 bp, Artificial Sequence
  Synthetic construct based on Agrobacterium tumefaciens sequence, Aequorea victoria sequence, Brassica napus sequence, Xanthomonas sequence, and Flavobacterium okeanokoites sequence
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgattacccatacgatgttccagattacgctatggctcctaagaagaagagaaaggttataacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagccgcggttcccgggagatcttggagggggcggtagcggcggtggcgggagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 30 - DNA, 7022 bp, Artificial Sequence
  Synthetic construct based on Agrobacterium tumefaciens sequence, Discosoma sp sequence, Brassica napus sequence, Xanthomonas sequence, and Flavobacterium okeanokoites sequence
cgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttcgaatacatctagatccaagtacatggcaaataatgattttattttgactgatagtgacctgttcgttgcaacaaattgatgagcaatgcttttttataatgccaactttgtatacaaaagttgtaggtacctcgcgaatgcatctagatccaatgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgataaggagactgccgctgccaagttcgagagacagcacatggacagcatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacgtacgtcgagcagcacgaggtggctgtggccagatactgcgacctccctagcaaactggggcacaaacttaatggagggggcggtagcggcggtggcgggagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagcattggatcggatcccgggcccgtcgactgcagaggcctgcatgcaaaccagctttcttgtacaaagttggcattataagaaagcattgcttatcaatttgttgcaacgaacaggtcactatcagtcaaaataaaatcattatttgtcgattgtcttcatcggatcccatcccctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggc

SEQ ID NO: 31 - DNA, 4190 bp, Artificial Sequence
  Synthetic construct based on Agrobacterium tumefaciens sequence, Discosoma sp sequence, Brassica napus sequence, Xanthomonas sequence, and Flavobacterium okeanokoites sequence
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctcgcccttacctgctagtcatgggcgatcctaaaaagaaacgtaaggtcatcgataaggagactgccgctgccaagttcgagagacagcacatggacagcatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacgtacgtcgagcagcacgaggtggctgtggccagatactgcgacctccctagcaaactggggcacaaacttaatggagggggcggtagcggcggtggcgggagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataactcgagaagggcgcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatggtcatagc

SEQ ID NO: 32 - DNA, 5149 bp, Artificial Sequence
  Synthetic construct based on Agrobacterium tumefaciens sequence, Aequorea victoria sequence, Arabidopsis thaliana sequence, Xanthomonas sequence, and Flavobacterium okeanokoites sequence
  Variation 3729, 3730: n is a, g, c, or t
tgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaacctggcttatcgaaattaatacgactcactatagggagcccggcagatctgatctcttgaactttccaagagttgaagaaaatcacagaaagccttagcacagagaagagagattgaagaagtcgacggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataaccatggagaggatatatatgtacatatgcaaagggatatcaagaccatctgtaatcttttgaagttttgtgaagctatagaagccaagcaagaattctaccagattacttcccaaataagtggtgtgaatgtaaattaataagagctacagaaacattgattggctcagtgtatgtgttgtattcatattcgttgttttattttatacggttgagaattgaataatgttgttgcatcaaatcactatgaaggacatttacagtcagctgctcgatcgaggcggccaacaacaaaaaaaaaaaaaaaaaaaaaaaaaaaaaaaaaaaaaaaaaaaaaaaaaaaaaaaaaaaaaaaagaaaagccaattgggatcnnagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttt

SEQ ID NO: 33 - DNA, 3001 bp, Artificial Sequence
  Synthetic construct based on Agrobacterium tumefaciens sequence, Aequorea victoria sequence, Arabidopsis thaliana sequence, Xanthomonas sequence, and Flavobacterium okeanokoites sequence
  Variation 2799, 2800: n is a, g, c, or t
taatacgactcactatagggagcccggcagatctgatctcttgaactttccaagagttgaagaaaatcacagaaagccttagcacagagaagagagattgaagaagtcgacggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataaccatggagaggatatatatgtacatatgcaaagggatatcaagaccatctgtaatcttttgaagttttgtgaagctatagaagccaagcaagaattctaccagattacttcccaaataagtggtgtgaatgtaaattaataagagctacagaaacattgattggctcagtgtatgtgttgtattcatattcgttgttttattttatacggttgagaattgaataatgttgttgcatcaaatcactatgaaggacatttacagtcagctgctcgatcgaggcggccaacaacaaaaaaaaaaaaaaaaaaaaaaaaaaaaaaaaaaaaaaaaaaaaaaaaaaaaaaaaaaaaaaaagaaaagccaattgggatcnnagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgctta

SEQ ID NO: 34 - DNA, 6381 bp, Artificial Sequence
  Synthetic construct based on Agrobacterium tumefaciens sequence, Discosoma sp sequence, Arabidopsis thaliana sequence, Xanthomonas sequence, and Flavobacterium okeanokoites sequence
  Variation 4961, 4962: n is a, g, c, or t
tgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttaacctggcttatcgaaattaatacgactcactatagggagcccggcagatctgatctcttgaactttccaagagttgaagaaaatcacagaaagccttagcacagagaagagagattgaagaagtcgacatgggcgatcctaaaaagaaacgtaaggtcatcgataaggagactgccgctgccaagttcgagagacagcacatggacagcatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacgtacgtcgagcagcacgaggtggctgtggccagatactgcgacctccctagcaaactggggcacaaacttaatggagggggcggtagcggcggtggcgggagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataaagaggatatatatgtacatatgcaaagggatatcaagaccatctgtaatcttttgaagttttgtgaagctatagaagccaagcaagaattctaccagattacttcccaaataagtggtgtgaatgtaaattaataagagctacgaaacattgattggctcagtgtatgtgttgtattcatattcgttgttttattttatacggttgagaattgaataatgttgttgcatcaaatcactatgaaggacatttacagtcagctgctcgatcgaggcggccaacaacaaaaaaaaaaaaaaaaaaaaaaaaaaaaaaaaaaaaaaaaaaaagaagagccaattgggatcnnagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttt

SEQ ID NO: 35 - DNA, 4017 bp, Artificial Sequence
  Synthetic construct based on Agrobacterium tumefaciens sequence, Discosoma sp sequence, Arabidopsis thaliana sequence, Xanthomonas sequence, and Flavobacterium okeanokoites sequence
taatacgactcactatagggagcccggcagatctgatctcttgaactttccaagagttgaagaaaatcacagaaagccttagcacagagaagagagattgaagaagtcgacatgggcgatcctaaaaagaaacgtaaggtcatcgataaggagactgccgctgccaagttcgagagacagcacatggacagcatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacgtacgtcgagcagcacgaggtggctgtggccagatactgcgacctccctagcaaactggggcacaaacttaatggagggggcggtagcggcggtggcgggagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataaagaggatatatatgtacatatgcaaagggatatcaagaccatctgtaatcttttgaagttttgtgaagctatagaagccaagcaagaattctaccagattacttcccaaataagtggtgtgaatgtaaattaataagagctacgaaacattgattggctcagtgtatgtgttgtattcatattcgttgttttattttatacggttgagaattgaataatgttgttgcatcaaatcactatgaaggacatttacagtcagctgctcgatcgaggcggccaacaacaaaaaaaaaaaaaaaaaaaaaaaaaaaaaaaaaaaaaaaaaaaagaaga

(19) United States
(12) Patent Application Publication
(10) Pub. No.: US 2023/0051935 A1
(43) Pub. Date: Feb. 16, 2023

(54) GENE EDITING USING A MESSENGER RIBONUCLEIC ACID CONSTRUCT

(71) Applicant: Calyxt, Inc., Roseville, MN (US)

(72) Inventor: SHAN Qiwei, Roseville, MN (US)

(73) Assignee: Calyxt, Inc., Roseville, MN (US)

(21) Appl. No.: 17/765,127

(22) PCT Filed: Sep. 30, 2020
(86) PCT No.: PCT/US2020/053469

§ 371 Date: Mar. 30, 2022

Related U.S. Application Data
(60) Provisional application No. 62/908,499, filed on Sep. 30, 2019.

(51) Int. Cl. C12N 15/82; C12N 9/22

(52) U.S. Cl. CPC C12N 15/8213; C12N 15/8209; C12N 9/22; C12N 2310/20

(57) ABSTRACT

Embodiments of the present disclosure are directed to a method that includes contacting a population of plant cells with a messenger ribonucleic acid (mRNA) construct including a sequence encoding a rare-cutting endonuclease and a detectable label, wherein the rare-cutting endonuclease is configured to induce a mutation at a target genomic locus. The method further includes screening the population of plant cells for the detectable label to identify target plant cells that are genetically transformed with the mRNA construct.

### INCORPORATION-BY-REFERENCE OF MATERIAL SUBMITTED ELECTRONICALLY

Incorporated by reference in its entirety is a computer-readable nucleotide/amino acid sequence listing, an ASCII text file which is 113 kb in size, submitted concurrently herewith, and identified as follows: “C1633108111_SequenceListing_ST25” and created on Sep. 29, 2020.

### BACKGROUND

Genome editing technologies using engineered nucleases, such as Transcription activator-like effector nucleases (TALEN), Clustered Regularly Interspaced Short Palindromic Repeats (CRISPR) and related CRISPER associated protein 9 (Cas9) or Cpf1 systems, have accelerated basic biology research, biotechnology, breeding, and gene therapy. Plant genome editing typically starts with transforming explant tissue with a deoxyribonucleic acid (DNA) genome editing vector either by Agrobacterium spp. or biolistic methods. Transformation is followed by tissue culture, including antibiotic or herbicide selection and regeneration of edited plantlets. The resulting primary generation plantlets are transgenic as exogenous nucleic acids are incorporated in the plant genome. For sexually reproducing plants, the transgene element can be segregated out in following generations by self-pollination or crossing with a wild-type plant. Such segregation efforts require significant time and resources to ultimately obtain plants without transgenes.

Scientists have tried several different methods to conduct genome editing without transgenic DNA integration. Non-transgenic approaches to gene editing are desirable for multiple reasons. Many plant species, especially root, tuber, and fruit bearing species including potato, strawberry, apple, grapes, and bananas are propagated asexually and can present a challenge for gene editing because exogenous nucleic acids cannot be removed by segregation. Previous approaches for non-transgenic gene editing are burdensome, require significant screening efforts to identify plants with the intended edits, and produce inconsistent results.

Accordingly, there remains a need for efficient techniques that allow for enrichment of gene edited events and that avoid exogenous DNA integration into the target cell genome.

### SUMMARY

The present disclosure is directed to overcoming the above-mentioned challenges and needs related to gene editing. This summary is provided to introduce a selection of concepts in a simplified form that are further described below in the Detailed Description.

In some embodiments, a method of gene editing comprises contacting a population of plant cells with a messenger ribonucleic acid (mRNA) construct including a sequence encoding a rare-cutting endonuclease and a detectable label. The rare-cutting endonuclease is configured to induce a mutation at a target genomic locus. The method further includes screening the population of plant cells for the detectable label to identify target plant cells that are genetically transformed with the mRNA construct.

In some embodiments, contacting the population of plant cells includes delivering the mRNA construct into the population of plant cells derived using at least one of polyethylene glycol (PEG) mediated transformation, electroporation, particle bombardment, and microinjection mediated protoplast transformation, as well as various combinations thereof.

In some embodiments, screening the population of plant cells for the detectable label includes isolating the target plant cells that have the detectable label from a remainder of the population of plant cells. In some embodiments, isolating the target cells includes using fluorescence activated cell sorting (FACS) with a nozzle having a diameter of at least 100 micrometers (um) and up to 200 um.

In some embodiments, the method further includes preparing the mRNA construct using in-vitro transcription, where the mRNA construct includes a TALEN mRNA including the sequence encoding the rare-cutting endonuclease and the detectable label.

In some embodiments, the rare-cutting endonuclease is a fusion protein and the sequence includes an endonuclease sequence encoding the rare-cutting endonuclease and a detectable label sequence encoding the detectable label. In some embodiments, the rare-cutting endonuclease includes a first half-TALEN that is labeled with a first detectable label and a second half-TALEN that is labeled with a second detectable label.

In some embodiments, the first detectable label and the second detectable label are different. In some embodiments, the first half-TALEN includes a first binding domain and a first endonuclease domain, and the first half-TALEN forms a first fusion protein with the first detectable label. In some embodiments, the second half-TALEN includes a second binding domain and a second endonuclease domain, and the second half-TALEN forms a second fusion protein with a second detectable label. The first detectable label and second detectable label can be label domains of the first and second fusion proteins, respectively. In some embodiments, the endonuclease domains and detectable label domains are separated by a flexible linker. In such embodiments, isolating the target plant cells from the population includes isolating the target plant cells that have or exhibit the first detectable label and the second detectable label.

In some embodiments, the detectable label sequence includes a fluorescent protein sequence. In some embodiments, the fluorescent protein is yellow fluorescent protein (YFP), red fluorescent protein (RFP), blue fluorescent protein (BFP), and the like.

In some embodiments, the rare-cutting endonuclease is conjugated to a detectable label. In some embodiments, the first half-TALEN is conjugated to a first detectable label and the second half-TALEN is conjugated to a second detectable label. In further embodiments, the first detectable label and the second detectable label are different. The detectable label can be a fluorophore, such as, Alexa Fluor 488, Alexa Fluor 647, Texas Red, FITC, or the like.

In some embodiments, the plant cells are plant protoplasts. In such embodiments, the method can further include culturing the target plant cells that are transformed with the mRNA construct and regenerating plants from the cultured target plant cells, where the regenerated plants express the mRNA construct.

Some embodiments are directed to a non-naturally occurring plant, generated by a genomic editing technique. In such embodiments, the genomic editing technique comprises contacting a population of plant cells with an mRNA construct that includes a sequence encoding a rare-cutting endonuclease and a detectable label. The rare-cutting endonuclease can be configured to induce a mutation at a target genomic locus. The genomic editing technique further includes screening the population of plant cells for the detectable label to identify target plant cells that are transformed with the mRNA construct, and regenerating a non-naturally occurring plant from the target plant cells. The mRNA construct can include an mRNA coding sequence including a rare-cutting endonuclease sequence encoding the rare-cutting endonuclease, and a detectable label sequence encoding the detectable label.

Some embodiments are directed to an mRNA construct comprising an mRNA coding sequence and a promoter sequence. The mRNA coding sequence includes a rare-cutting endonuclease sequence and a detectable label sequence. The promoter sequence is upstream from the mRNA coding sequence. The promoter sequence can be operatively linked to the rare-cutting endonuclease sequence.

In some embodiments, the mRNA construct further includes a first untranslated region (UTR) upstream from the mRNA coding sequence and downstream from the promoter sequence. In some embodiments, the mRNA construct further includes a second UTR downstream from the mRNA coding sequence.

In some embodiments, the rare-cutting endonuclease sequence includes a sequence encoding a TALEN. For example, the rare-cutting endonuclease sequence can encode a binding domain and an endonuclease domain of the TALEN.

In some embodiments, the detectable label includes a first detectable label and a second detectable label, and the rare-cutting endonuclease includes a first half-TALEN that is labeled with the first detectable label and a second half-TALEN that is labeled with the second detectable label. In some embodiments, the first detectable label and the second detectable label are different.

In some embodiments, the first half-TALEN includes a first binding domain and a first endonuclease domain that forms a first fusion protein with the first detectable label. In such embodiments, the second half-TALEN includes a second binding domain and a second endonuclease domain that forms a second fusion protein with a second detectable label. The first detectable label can be a first label domain of the first fusion protein and the second detectable label can be a second label domain of the second fusion protein. In some embodiments, the first detectable label and the second detectable label each include a fluorescent protein.

In some embodiments, the first half-TALEN is conjugated to the first detectable label, and the second half-TALEN is conjugated to the second detectable label.

In some embodiments, the rare-cutting endonuclease sequence and the detectable label sequence are separated by a flexible linker sequence.

In some embodiments, the detectable label sequence includes a detectably labeled nucleotide. In further embodiments, the detectably labeled nucleotide includes a fluorophore.

In some embodiments, the plant cells are plant protoplasts.

In some embodiments, the plant cells are, or are derived from, protoplasts, callus, immature embryos, somatic embryos, embryo axis, meristematic tissue, leaf tissue, stem tissue, or root tissue.

In some embodiments, the plant cells are dicotyledonous plant cells. In some embodiments, the dicotyledonous plant cells are soybean, canola, alfalfa, potato, and the like. In other embodiments, the plant cells are monocotyledonous plant cells. In some embodiments, the monocotyledonous plant cells are corn, wheat, oats, and the like.

### BRIEF DESCRIPTION OF THE DRAWINGS

Various example embodiments can be more completely understood in consideration of the following detailed description in connection with the accompanying drawings, in which:

FIG. 1 is a flow diagram illustrating an example method for gene editing a population of plant cells, consistent with the present disclosure.

FIGS. 2A-2B are diagrams illustrating example mRNA constructs, consistent with the present disclosure.

FIGS. 3A-3F are diagrams illustrating example mRNA coding sequences of mRNA constructs, such as the mRNA constructs illustrated by FIGS. 2A-2B, consistent with the present disclosure.

FIG. 4 is a flow diagram illustrating an example method for gene editing a population of plant cells, consistent with the present disclosure.

FIG. 5 is a flow diagram illustrating another example method for gene editing a population of plant cells, consistent with the present disclosure.

FIGS. 6A-8C illustrate example flow cytometry data demonstrating sorting of protoplasts transformed using the nucleic acid constructs of Table 2, consistent with the present disclosure.

FIGS. 9A-9D illustrate microscopy images of plant cells transformed using the nucleic acid constructs of Table 4, consistent with the present disclosure.

FIG. 10 illustrates detected deletions of plants transformed using the nucleic acid constructs of Table 4, consistent with the present disclosure.

### DETAILED DESCRIPTION

Aspects of the present disclosure are directed to a variety of methods, constructs, and plants involving and/or developed using non-DNA constructs that encode rare-cutting endonucleases and a detectable label. These methods include direct delivery of RNA and/or protein to the plant cells. Example embodiments include contacting a population of plant cells with an mRNA construct to transform the plant cells. The mRNA construct encodes the rare-cutting endonuclease and the detectable label, and the rare-cutting endonuclease can induce a mutation at a target genomic locus. The contacted population of plant cells can be screened for cells with the mutation at the target genomic locus. While the present invention is not necessarily limited to such applications, various aspects of the invention may be appreciated through a discussion of various embodiments using this context.

Accordingly, in the following description various specific details are set forth to describe specific embodiments presented herein. It should be apparent to one skilled in the art, however, that one or more other examples and/or variations of these embodiments can be practiced without all the specific details given below. In other instances, well known features have not been described in detail so as not to obscure the description of the embodiments herein. For ease of illustration, the same reference numerals can be used in different diagrams to refer to the same elements or additional instances of the same element.

Plant transformation and tissue culture present significant limitations to genome editing efforts and are costly in terms of time, labor and materials to develop and implement specialized protocols. Non-DNA gene editing, sometimes herein referred to as “DNA-free editing”, typically requires time-consuming and expensive dedicated protocols to generate and deliver reagents but can save time by not requiring incorporation of transgenic DNA. Methods consistent with embodiments of the present disclosure can include delivering an in vitro-purified mRNA construct into plant tissues or plant cells derived from plant tissues. The mRNA construct includes the non-DNA gene editing reagents, such as the encoded rare-cutting endonuclease, and a detectable label used to identify plant cells and/or plant tissue transformed by and/or including the mRNA construct. The plant cells transiently exposed to the non-DNA gene editing reagents can be screened to identify plant cells and/or plant tissue transformed by and/or that include the mRNA construct through physical means, such as FACS. The plant cells that contain the intended gene edit(s) can be separated from the remainder of the plant cell population. Example methods in accordance with the present disclosure can reduce the laborious process of screening for desired mutations or edits. In some embodiments, example methods directed to gene edits on sexually reproduced plants or other types of plants can avoid any requirement for imposed segregation and avoid transformants that include DNA integrations into the genome.

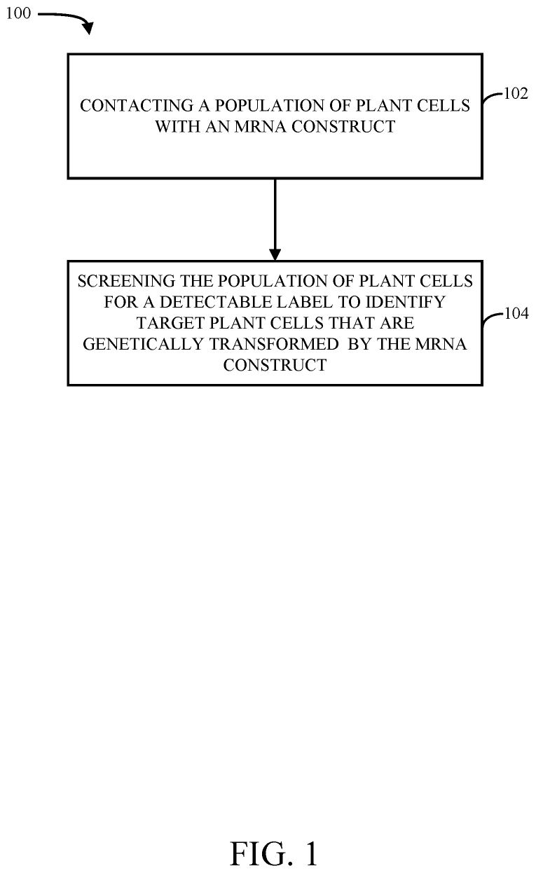

Turning now the figures, FIG. 1 is a flow diagram illustrating an example method 100 for gene editing a population of plant cells, consistent with the present disclosure. The plant cells can be derived from a variety of different types of plants and/or plant tissue. As non-limiting examples, the plant cells can include and/or can be derived from protoplasts, callus, immature embryos, somatic embryos, embryo axis, meristematic tissue, leaf tissue, stem tissue, root tissue, etc. The plants can include dicotyledonous plants and plant cells, such as soybean, canola, alfalfa, potato, and the like, as well as monocotyledonous plants and plant cells, such as corn, wheat, oats, and the like.

At 102, the method 100 includes contacting a population of plant cells with an mRNA construct. As used herein, an mRNA construct includes and/or refers a nucleic acid sequence including one or more binary vectors carrying genome editing reagents, a detectable label, and a promoter. The genome editing reagents can include or encode an endonuclease, such as a TALEN mRNA. For example, the mRNA construct includes a sequence encoding a rare-cutting endonuclease and a detectable label. The rare-cutting endonuclease can include a TALEN and related Fok1 protein, or CRISPR and related Cas9 or Cpf1, among other endonucleases. The detectable label can include a fluorescent protein, a fluorophore, or nucleotide bound to a fluorophore, among other types of labels. In some embodiments, the rare-cutting endonuclease is a TALEN that includes an endonuclease domain and a binding domain (sometimes referred to as a “TALE domain”). The binding domain can be configured to bind a target location and the endonuclease domain is configured to induce a mutation at a target genomic locus associated with the target location.

As used herein, a domain includes and/or refers to a conserved part of a protein sequence and tertiary structure of the protein that can form a three-dimensional structure. The domains can be encoded by the mRNA constructs, as further described below.

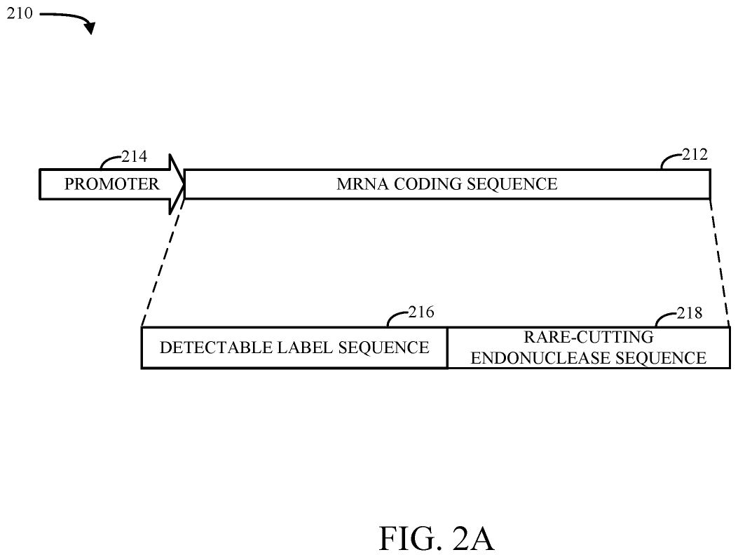

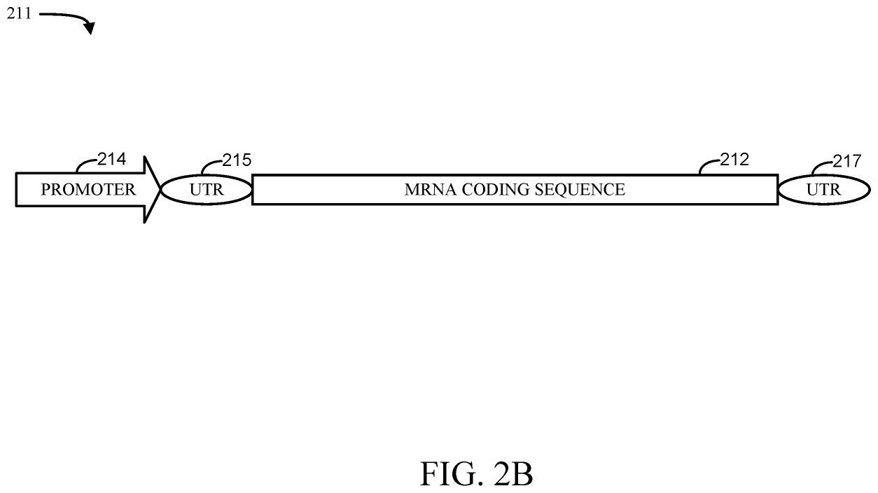

The mRNA construct can include a variety of nucleic acid segments, selected and arranged to facilitate transport of genome editing reagents in the plant cells. For instance, the mRNA construct can include a TALEN mRNA that includes the sequence encoding the rare-cutting endonuclease and the detectable label. In some embodiments, the mRNA construct includes an mRNA coding sequence, a UTR, and the promoter sequence. The UTR can be upstream from the mRNA coding sequence, such as a 5′ UTR. In some embodiments, the mRNA construct can include the mRNA coding sequence, the promoter sequence, and a UTR downstream from the mRNA coding sequence, such as a 3′ UTR. In various embodiments, the mRNA construct can include the mRNA coding sequence, a first UTR upstream from the mRNA coding sequence (e.g., a 5′ UTR), a second UTR downstream from the mRNA encoding sequence (e.g., a '3 UTR), and a promoter sequence that is upstream the first UTR. Example mRNA constructs are illustrated in FIGS. 2A-2B and discussed further herein.

Example mRNA constructs in accordance with the present disclosure can have a variety of forms, as further illustrated herein. In some embodiments, the detectable label can include a nucleotide of the mRNA construct that is labeled with a fluorophore. In some embodiments, a plurality of nucleotides of the mRNA construct are labeled with a fluorophore.

Contacting the population of plants cells with the mRNA construct can include delivering the mRNA construct into the population of plant cells. The mRNA construct can be delivered into the plant cells via different approaches including, but not limited to, PEG-mediated transformation, electroporation, particle bombardment, or microinjection mediated protoplast transformation, as well as combinations thereof. Specific examples of the delivery approaches are further described below.

In various embodiments, prior to contacting a population of plant cells with the mRNA construct at 102, the method 100 can include preparing the mRNA construct using in-vitro transcription. For example, the gene editing reagents can be prepared as a DNA vector that encodes the rare-cutting endonuclease and a promotor to stimulate transcription. In some embodiments, the DNA vector further encodes the detectable label. The gene editing reagents can be mixed with RNA nucleotides and polymerase in a tube and purified, resulting in transcription of the DNA vector to an mRNA construct. In some embodiments, rather than the DNA vector encoding the detectable label, one or more nucleotides of the mRNA construct can be labeled, such as with a fluorophore.

At 104, the method 100 includes screening the population of plant cells for the detectable label to identify target plant cells that are genetically transformed with the mRNA construct. Target plant cells, as used herein, include and/or refer to plant cells that express the mRNA construct and/or that otherwise exhibit or express the detectable label. The target plant cells can include the intended mutation at the target genomic locus. In some embodiments, the population of plant cells can be screened and target plant cells can be selected for expression of the mRNA construct via the detectable label. Screening the population of plant cells for the detectable label can include isolating target plant cells that have the detectable label from a remainder of the population of plant cells. Various embodiments include FACS based selection of transformed protoplasts. As further described below, isolating target cells can include using FACS with a nozzle having a diameter of at least 100 um and up to 200 um.

FACS applied to plant protoplasts can be difficult because maintaining live protoplasts after sorting is challenging, plant regeneration from protoplasts is difficult to perform, and debris generated during enzymatic treatment of plant tissue can clog the instrument and hinder the FACS process. For example, with no cell wall for protection, protoplasts are extremely fragile during transportation and sorting. Somewhat surprisingly, various embodiments of the present disclosure include implementing FACS protocols that successfully segregate transformed plant protoplasts and allow for plant regeneration. Method embodiments in accordance with the present disclosure can include a FACS based screening or selection of protoplasts using a 100-200 um diameter nozzle to reduce pressure on the protoplasts as compared to smaller nozzles, such as 85 um and 70 um nozzles. In some specific embodiments, the nozzle can have a diameter of between 100-150 um, between 100-130 um, or between 120-130 um. In more specific embodiments, the nozzle diameter is 120 um, 130 um, 150 um, or 200 um. The larger nozzle size can reduce sorting speed as compared to the smaller nozzles. For example, the larger nozzle size can reduce the sorting speed by about 2-5 million events per hour as compared to the smaller nozzles. However, larger nozzle size can provide increased stability and viability.

In some embodiments, the detectable label includes a first detectable label and a second detectable label. The rare-cutting endonuclease can include a first half-TALEN (e.g., left-half TALEN (LHT)) that is labeled with the first detectable label and a second half-TALEN that is labeled with the second detectable label (e.g., right-half TALEN (RHT)). In such embodiments, the method 100 can further include isolating the target plant cells that have the first detectable label and the second detectable label. In some embodiments, the first detectable label and second detectable label can be different labels. In other embodiments, the first detectable label and second detectable label can be the same. Although embodiments are not so limited, and the mRNA construct can encode and/or the rare-cutting endonuclease can be labeled with a single detectable label and/or more than two detectable labels. In some embodiments, the mRNA construct itself can be labeled with a fluorophore.

Accordingly, a number of embodiments are directed to the combination of non-DNA-mediated plant cell editing of protoplast plant cells, along with the selection of target cells receiving both half TALENs using FACS and fluorescent proteins or fluorophore labelling of the two TALENs. Such a combination can allow for a highly efficient method to overcome the obstacle of a non-DNA editing method, where use of traditional selectable markers cannot be employed. Plants regenerated from FACS selected protoplasts can enriched for the intended gene edits, thus reducing the screening efforts typically required with transient gene expression.

As described above, the individual half TALEN constructs can contain the detectable labels. For example, the individual half TALEN constructs can be fusion proteins that contain fluorescent protein domains, with or without intervening flexible linker domains. Example detectable labels, such as fluorescent proteins, can be incorporated into such a fusion protein. Non-limiting examples of fluorescent proteins include YFP, RFP, and BFP, among others. Although examples are not so limited, and other fluorescent proteins can be used, such as cyan-linker yellow (CLY).

In various embodiments, the first individual half TALEN construct has a fluorescent protein domain, such as YFP, attached at the N-terminus of the left half TALEN (LHT) separated with a peptide linker, such as GGGGSGGGGS. In such embodiments, the corresponding other individual half TALEN construct has a fluorescent protein, such as RFP attached at the N-terminus of the right half TALEN (RHT) separated with a flexible (peptide) linker, such as GGGGSGGGGS. To improve the mRNA stability and overall expression, UTR sequences, e.g., from the Arabidopsis gene At1G09740, can be added, flanking the TALEN coding sequences. These expression cassettes can be used for in-vitro transcription to obtain high-quality purified mRNA encoding the TALEN subunits, or for protein expression and purification in a bacterial or insect cell expression system using standard methods.

In some embodiments, instead of creating fusion proteins with detectable label domains, the purified nuclease proteins can be labeled by a conjugation-based method with a commercial labeling kit such as Alexa Fluor 488 Protein Labeling Kit (Thermo Fisher Scientific, Cat #A10235).

In some embodiments, the mRNA encoding the nuclease can itself be chemically labeled by incorporating labeled nucleotides into the mRNA during the in vitro transcription process. This incorporation-based labeling method can achieve uniformity and consistency in labeling the mRNA. For example, fluorophore-labeled ChromaTide™ (Thermo Fisher Scientific) uridine-5′-triphosphates (UTPs) can be enzymatically incorporated into RNA or probes. Cells transformed with the labeled mRNA can then be detected.

The present disclosure addresses contamination problems through use of antibiotics and fungicides in liquid media, frequent media changes after sorting, and cell sorter sterilization using bleach and ethanol. For example, embodiments in accordance with the present disclosure can avoid the use of antibiotics and/or fungicides as transformed cells are selected based on a detectable label, and not based on resistant gene expression to an antibiotic and/or fungicide. Table 3 as further illustrated herein is an example of FACS canola protoplasts with nucleic acid vectors that include a fluorescent protein, such as a fluorescent protein expression DNA vector.

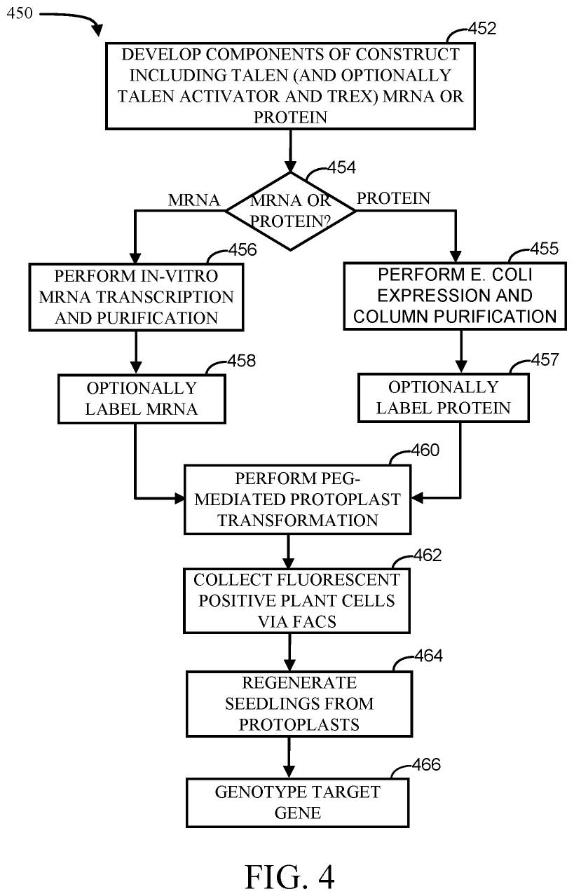

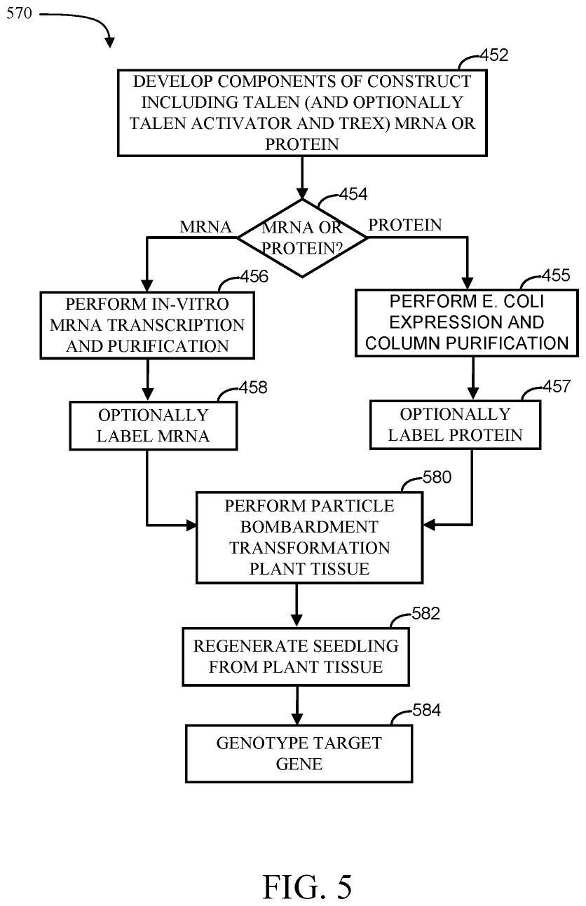

Various embodiments of the present disclosure are directed to a non-naturally occurring plant generated by the method 100 described by FIG. 1 and/or the methods 450, 570 described further herein by FIGS. 4-5. For example, the method 100 can further include culturing the identified target plant cells that are transformed with the mRNA construct, and regenerating plants from the cultured target plant cells, where the regenerated plants express the mRNA construct. The plants can be generated using example mRNA constructs, such as those illustrated by FIGS. 2A-2B.

In some embodiments and consistent with method 100, a non-naturally occurring plant can be generated by a genomic editing technique that includes using an mRNA construct. The mRNA construct can include a rare-cutting endonuclease sequence which encodes the rare cutting endonuclease and a detectable label sequence which encodes or includes the detectable label. The genomic editing technique can include contacting a population of plant cells with the mRNA construct, screening the population of plant cells for the detectable label to identify target plant cells that are transformed with the mRNA construct, and regenerating a non-naturally occurring plant from the identified target plant cells. Other example embodiments of the disclosure are directed to naturally occurring seed, reproductive tissue, or vegetative tissue generated by the method 100 of FIG. 1.

FIGS. 2A-2B are diagrams illustrating example mRNA constructs 210, 211, consistent with the present disclosure. As shown by FIG. 2A, the mRNA construct 210 includes an mRNA coding sequence 212 and a promoter sequence 214 upstream from the mRNA coding sequence 214. As non-limiting examples, the promoter can include a nopaline synthase promoter (NosPro) or a T7 promoter, among others. Other example promoters can include Sp6 promoter, a T3 promoter, Ubi promoter, a CaMV35S promoter, an ADHI promoter, and ADH1 promoter, a GDS promoter, a TEF1 promoter, a Gall promoter, a CaMKlla promoter, a T7lac promoter, an araBAD promoter, a trp promoter, a lac promoter, a Ptac promoter, among others.

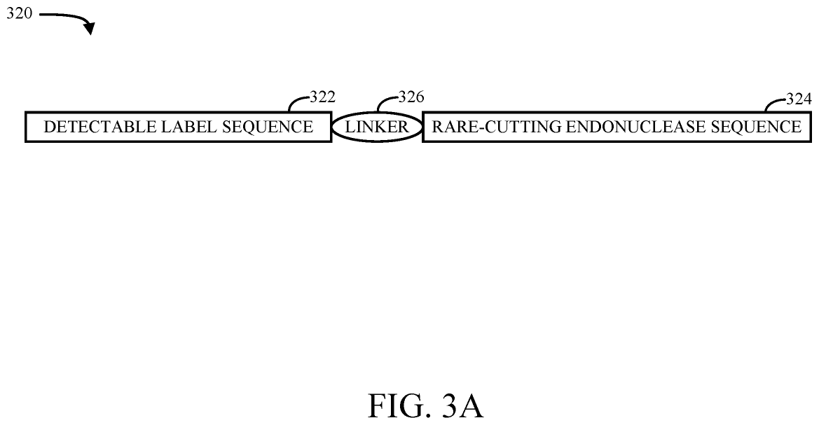

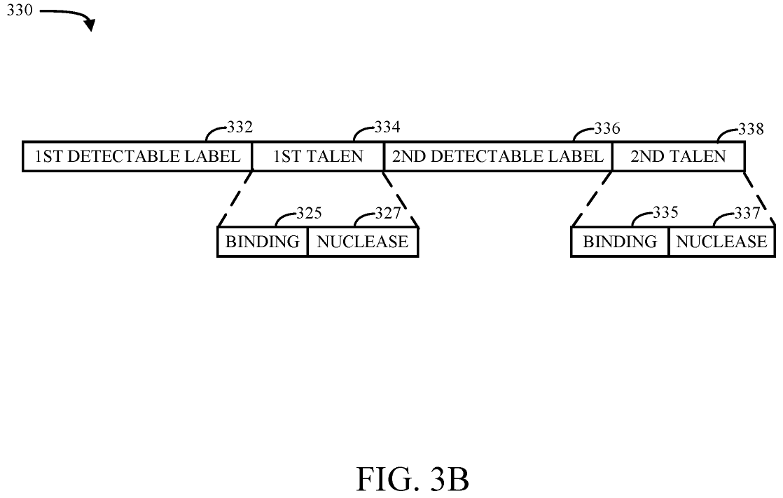

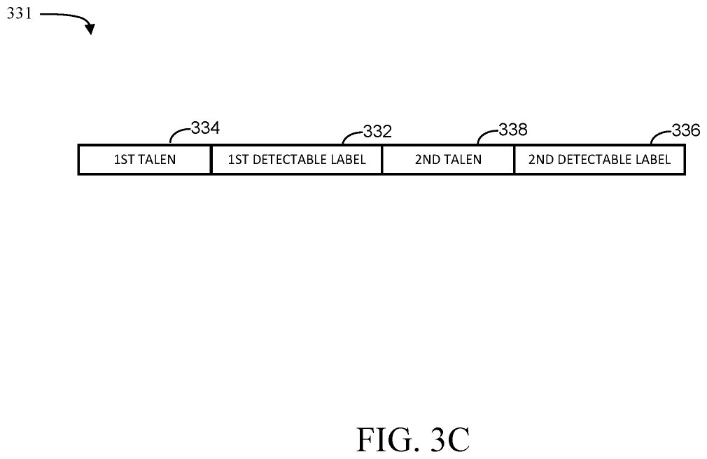

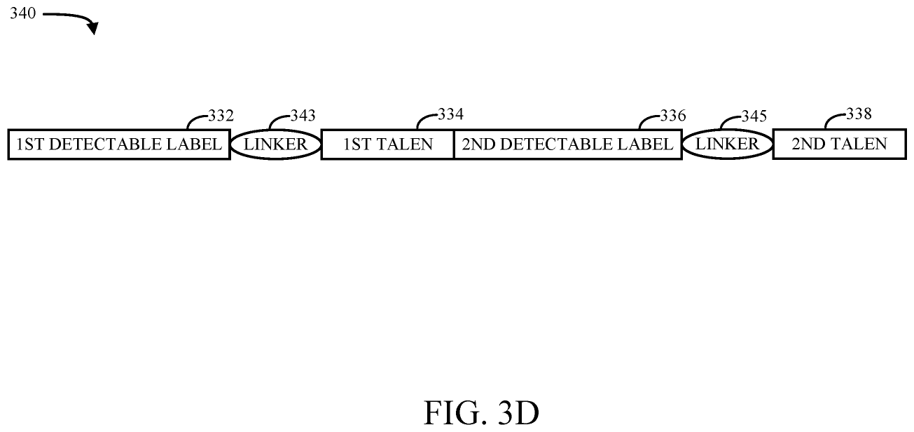

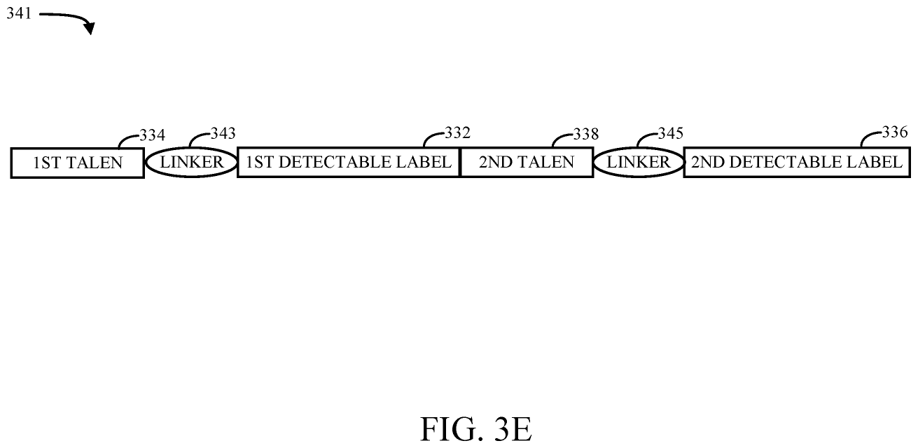

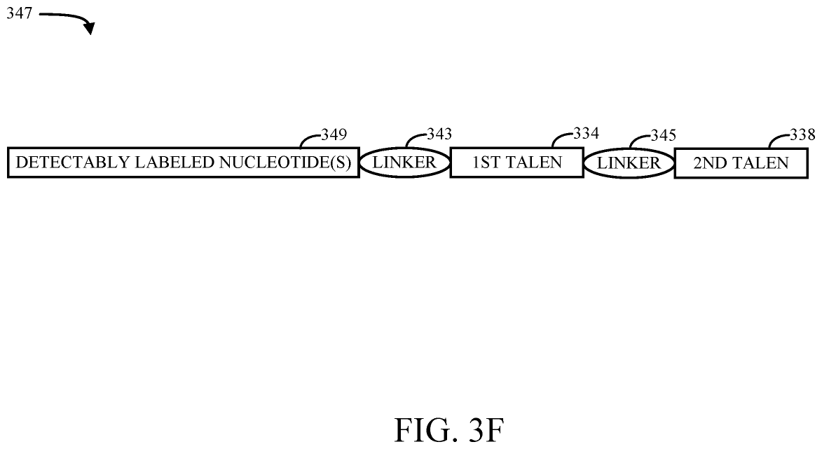

The mRNA coding sequence 212 can include a detectable label sequence 216 and a rare-cutting endonuclease sequence 218. As further illustrated by FIGS. 3A-3F, the rare-cutting endonuclease sequence 218 can include a sequence encoding a TALEN. In some embodiments, the rare-cutting endonuclease sequence 218 can encode a binding domain and endonuclease domain. The binding domain can be configured to bind to a target sequence. The rare-cutting endonuclease domain can be configured to induce a mutation at a target genomic locus associated with the target location. However, embodiments are not so limited. The detectable label sequence 216 encodes or includes the detectable label, such as a fluorescence protein sequence, a fluorophore, and/or a nucleotide (e.g., an RNA nucleotide) that is labeled with a fluorophore, as further described herein.

In the embodiments illustrated by FIG. 2A, the detectable label sequence 216 is upstream from the rare-cutting endonuclease sequence 218. However, embodiments are not so limited, and the rare-cutting endonuclease sequence 218 can be upstream from the detectable label sequence 216. As may be appreciated, upstream can include a location proximal to and/or closer to the 5′ end of the mRNA construct 210 and/or mRNA coding sequence 212 as compared to the referenced sequence. Conversely, downstream can include a location proximal to and/or closer to the 3′ end of the mRNA construct 210 and/or mRNA coding sequence 212 as compared to the referenced sequence. As used herein, a sequence with adjectives listed in front, such as the detectable label sequence 216 and the rare-cutting endonuclease sequence 218, includes or refers to a nucleotide sequence that encodes or is the adjectives (e.g., encodes or is the detectable label).

In some embodiments and as shown by the mRNA construct 211 of FIG. 2B, the promoter sequence 214 can be upstream from the mRNA coding sequence 212, and at least one UTR 215, 217 can be downstream from the promoter sequence 214 and upstream from the mRNA coding sequence 212. For example, the mRNA coding sequence 211 of FIG. 2B includes a first UTR 215 upstream from the mRNA coding sequence 212, and the promoter sequence 214 is upstream the first UTR 215. In some embodiments, the mRNA construct 211 includes a second UTR 217 that is downstream from the mRNA coding sequence 212. However, embodiments are not so limited, and additional and/or different mRNA constructs are contemplated. For example, the mRNA construct can include no UTR and/or a single UTR as described above.

As further shown and described by FIGS. 3A-3F, the mRNA coding sequence of example mRNA constructs can have a variety of forms. In a number of embodiments, the detectable label sequence 216 and the rare-cutting endonuclease sequence 218 can form a fusion protein when translated. In some embodiments, the detectable label sequence 216 includes a nucleotide of the mRNA construct that is detectably labeled, such as with a fluorophore.

FIGS. 3A-3F are diagrams illustrating example mRNA coding sequences of mRNA constructs, such as the mRNA constructs illustrated by FIGS. 2A-2B, consistent with the present disclosure. Each of the mRNA coding sequences illustrated by FIGS. 3A-3F include the detectable label sequence 216 and the rare-cutting endonuclease sequence 218 as illustrated by FIGS. 2A-2B.

In some embodiments and as shown by FIG. 3A, the mRNA coding sequence 320 can include the detectable label sequence 322 and the rare-cutting endonuclease sequence 324 which are separated by a flexible linker sequence 326. The flexible linker sequence 326 can include a plurality of nucleotides. For example, the flexible linker sequence 326 can encode a flexible peptide linker. As shown by FIG. 3A, the detectable label sequence 322 can be upstream from the rare-cutting endonuclease sequence 324, however embodiments are not so limited and the detectable label sequence 322 can be downstream from the rare-cutting endonuclease sequence 324.

FIG. 3B illustrates an example mRNA coding sequence 330 that includes a first half-TALEN sequence 334 and a second half-TALEN sequence 338, which can encode a LHT and a RHT. In some examples, the detectable label sequence can include a first detectable label sequence 332 that labels the first half-TALEN (e.g., the first half-TALEN sequence 334) and a second detectable label sequence 336 that labels the second half-TALEN (e.g., the second half-TALEN sequence 338). As previously described, the first detectable label encoded by the first detectable label sequence 332 and the second detectable label encoded by the second detectable label sequence 336 can be different, such as sequences encoding different florescent proteins and/or fluorophores.

Each of the first half-TALEN sequence 334 and second half-TALEN sequence 338 can encode a binding domain 325, 335 and an endonuclease domain 327, 337. In some embodiments, the half-TALEN sequences 334, 338 and the detectable label sequences 332, 336 can form and/or encode a first fusion protein and a second fusion protein. For example, the first half-TALEN sequence 334 can encode a first binding domain 325 and a first endonuclease domain 327 that form a first fusion protein with the first detectable label encoded by the first detectable label sequence 332 when translated. The second half-TALEN sequence 338 can encode a second binding domain 335 and a second endonuclease domain 337 that form a second fusion protein with the second detectable label encoded by the second detectable label sequence 336 when translated.

The mRNA coding sequence 330 of FIG. 3B illustrates the detectable label sequences 332, 336 upstream from the TALEN sequences 334, 338, respectively. However, embodiments are not so limited. For example, FIG. 3C illustrates an example mRNA coding sequence 331, which is similar to the mRNA coding sequence 330 but with the first half-TALEN sequence 334 upstream of the first detectable label sequence 332 and the second half-TALEN sequence 338 upstream of the second detectable label sequence 336.

As previously described, the rare-cutting endonuclease sequence and detectable label sequence can be separated by a flexible linker sequence which encodes or includes a flexible linker. FIG. 3D illustrates an example of an mRNA construct 340 which is similar to the mRNA coding sequence 330 of FIG. 3D with the addition of flexible linker sequences 343, 345 between the detectable label sequences 332, 336 and the half-TALEN sequences 334, 338. For example, the mRNA construct 340 includes a first detectable label sequence 332 and a first half-TALEN sequence 334 that are separated by a first flexible linker sequence 343. The mRNA construct 340 can further include a second detectable label sequence 336 and a second half-TALEN sequence 338 that are separated by a second flexible linker sequence 345. Although not illustrated, the first half-TALEN sequence 334 and the second detectable label sequence 336 can be separated by a third flexible linker sequence.

The mRNA coding sequence 340 of FIG. 3C illustrates the detectable label sequences 332, 336 upstream from the TALEN sequences 334, 338, respectively. However, embodiments are not so limited. For example, FIG. 3E illustrates an example mRNA coding sequence 341, which is similar to the mRNA coding sequence 340 but with the first half-TALEN sequence 334 upstream of the first detectable label sequence 332 and the second half-TALEN sequence 338 upstream of the second detectable label sequence 336. Similarly, although not illustrated, the first detectable label sequence 332 and the second half-TALEN sequence 338 can be separated by a third flexible linker sequence.

FIG. 3F illustrates an example mRNA coding sequence 347 in which the detectable label sequence includes a detectably labeled nucleotide 349. As shown, the mRNA coding sequence 347 includes the detectably labeled nucleotide 349 which is upstream from the first half-TALEN sequence 334 and the second half-TALEN sequence 338. For example, detectably labeled nucleotide 349 can include a nucleotide of the mRNA construct that is bound to a fluorophore or other detectable label. Although embodiments are not so limited and the detectably labeled nucleotide 349 can be downstream of the second half-TALEN sequence 338. In some embodiments, at least one flexible linker sequence 343, 345 can separate the detectably labeled nucleotide 349 from the first half-TALEN sequence 334 and/or separate the first half-TALEN sequence 334 from the second half-TALEN sequence 338. As may be appreciated, the detectably labeled nucleotide 349 can include a plurality of detectably labeled nucleotides, which can increase the signal strength of the detectable label as compared to a single detectably labeled nucleotide.

Different example approaches for enriching and/or screening the plant cells for the intended gene edit(s) are now described. Enriching and/or screening the plant cells can increase the representation of plant cells likely to contain the intended genomic edit.

FIG. 4 is a flow diagram illustrating an example method 450 for gene editing a population of plant cells, consistent with the present disclosure. At 452, the method 450 can include developing components of the construct (e.g., mRNA or protein). For an mRNA construct, the components can include the TALEN vector, such as a sequence including a TALEN, a Fusion Protein (FP)-TALEN, a detectable label, a TALE-activator and/or Trex2. Similar components can be prepared for a protein construct. The components can be prepared separately by different techniques. At 454, the method can include identifying whether the construct is an mRNA construct or protein construct. As may be appreciated, step 454 may not occur but is shown to illustrate that different method steps can occur for the developing an mRNA construct or a protein construct. In response to a determination that the construct is an mRNA construct, the method 450 at 456 includes performing in-vitro mRNA transcription and purification, as previous described. At 458, the method 450 optionally includes labeling the mRNA construct with chemical dyes, such as to increase a signal strength of the detectable label and/or to label the nucleotide(s) to include or form the detectable label(s). In some embodiments, in response to determining the construct is a protein construct, at 455, the method 450 includes performing E. coli expression of the protein and column purification. At 457, the method can optionally include labeling the protein construct with chemical dyes, similar to the mRNA construct as described above.

At 460, the method 450 includes performing PEG-mediated protoplast transformation using the mRNA construct or protein construct. After a period of time, such as around twenty-four hours, at 462, the protoplasts can be sorted with FACs for fluorescent positive cells. At 464, the method 450 can further include collecting the positive cells by culturing on liquid and solid mediums and regenerating into plants. At 466, the plants can be screened by genotyping for the mutation of the target gene.

In some specific embodiments, the PEG-mediated transformation can start with the isolation of protoplasts from healthy plant tissues that are regenerable, for example, canola young leaf blade, wheat immature embryos, or soybean somatic embryos, embryo axis etc. Next, the tissues can be digested in buffer with enzymes such as cellulose, macerozyme (and/or) pectolyase. After a few hours of digestion, round and intact protoplasts can be isolated in a first buffer, such as mannitol magnesium (MMG), for transformation. The mRNA/protein reagents (e.g., the mRNA construct) can be added into a tube with protoplasts and polyethylene glycol, such as 40% PEG4000. The tube is mixed and incubated, such as for 20-30 minutes. The protoplasts can be washed with a second buffer (e.g., W5 buffer) and transferred into a third buffer (e.g., M8P buffer). The TALENs can be fused with a detectable label, such as a fluorescent protein. After incubation (such as for 16-36 hours), the fluorescent signal can be detected under microscope and/or FACS. If the mRNA construct or protein are labeled with chemical dyes, the mRNA construct or protein can be sorted after transformation. Fluorescent positive cells are collected and transferred into regeneration medium. The protoplasts can be cultured in several rounds of liquid medium, then moved to callus inducing medium (CIM), shoot inducing medium (SIM) and rooting medium (RM).

Although FIG. 4 illustrates use of PEG-mediated transformation, embodiments are not so limited. In some embodiments, fluorescently labeled TALEN constructs (mRNA and/or protein constructs) are delivered into plant protoplast cells or other tissues using other methods such as electroporation, bombardment, or microinjection mediated protoplast transformation. For larger plant tissues with cell walls such as embryos, bombardment (or biolistics) with gold particles coated with mRNA can be used as delivery methods. Following delivery of the fluorescently labeled endonucleases, e.g., mRNA constructs encoding the endonucleases, FACS can be used to select fluorescent colored positive protoplast cells. In embodiments where two differentially-labeled half TALEN constructs are used, FACS can be used to select dual fluorescent colored positive protoplast cells. And, the selected protoplasts can be regenerated into whole plants, as described above.

For particle bombardment transformation, the mRNA constructs or proteins can be coated onto particles, such as gold particles. To coat the mRNA or protein(s) on the gold particles, different volumes of mRNA or protein solution are mixed with a fixed amount of gold suspension by pipetting.

Ammonium acetate and 2-propanol can be used to precipitate the mRNA TALEN onto gold particles. For example, the following protocol can be used:

2 microliters (μl) of TALEN mRNA 1 μl Left half TALEN at 1 micrograms (μg)/μl, and 1 μl Right half TALEN at 1 μg/μl) and

1 μl of TALE-activator (1 μg/μl),

1 μl Ammonium acetate (5 moles (M)),

20 μl 2-propanol, and

5 μl gold nanoparticles (40 milligrams (mg)/milliliter (ml) for single delivery.

For protein bombardment, the following example protocol can be used:

2 μl of TALEN protein (1 μl Left half TALEN at 2 μg/μl, and 1 μl Right half TALEN at 2 μg/μl),

1 μl of TALE-activator (2 μg/μl), and

5 μl gold nanoparticles (40 mg/ml) for one delivery.

A PDS-1000/He gene gun (Bio-Rad) can be used according to general settings. Various embodiments include at least substantially the same features and attributes, include Bio-Rad settings, as discussed within Kikkert, et al. Plant Cell, Tissue and Organ Culture, volume 33, pages 221-226 (1993), which is hereby incorporated by reference in its entirety for its general teachings related to Bio-Rad the specific teachings related to example general settings for Bio-Rad.

Although embodiments are not so limited, and various particle bombardment transformation protocols can be used.

In some embodiments, the detectably labeled endonuclease or the detectably labeled mRNA construct encoding the nuclease can be co-delivered with an in vitro purified exonuclease or mRNA encoding the exonuclease. An example exonuclease is Trex2. Co-delivery of an exonuclease (or an encoding mRNA) and the mRNA construct can increase the efficiency of non-homologous end joining (NHEJ)-mediated deletions at the endonuclease target cutting site, thus further increasing the likelihood and/or the efficiency of the deletion. Some embodiments include the triple co-delivery of the endonuclease reagent (e.g., TALEN), an exonuclease (e.g., Trex2), and a TALE-activator (as further described herein) to further increase efficiency (e.g., frequency) in inducing deletions.

FIG. 5 is a flow diagram illustrating another example method for gene editing a population of plant cells, consistent with the present disclosure. The method 570 can include steps 452, 454, 456, 458, 455, and 457 as previously described by method 450, and which are not repeated herein. At 580, the method 570 includes delivering the mRNA or protein construct by performing particle bombardment transformation. At 582, the plant tissues can be cultured on solid mediums and regenerated into plants. And, at 584, the plants can be screened by genotyping for the mutation of the target gene.

In some embodiments, in addition to contacting the population of target cells with an mRNA or protein construct including a sequence encoding the rare-cutting endonuclease, the method 570 (or method 550) further includes contacting the population of target cells with an agent that confers a selective advantage on transiently transformed cells. By conferring a selective advantage, co-administration of the additional agent promotes enhanced growth and proliferation of cells that are transformed with the non-DNA gene editing reagents (see, e.g., Table 3, which indicates this effect). In some embodiments, the agent that confers a selective advantage includes a TALE activator. The TALE activator can include a TALE DNA binding domain (e.g., a TALEN reagent) and an activator agent. Example activator agents include TALE-VP128, 6TAD and a 6TAD-VP128 fusion. Example activator agents include nucleotide and amino acid sequences set forth in SEQ ID NOs: 22-27. The TALE DNA binding domain (e.g., a TALEN reagent) and the TALE-activator together target genes that promote morphogenic traits. These morphogenic traits can include hormone regulators that regulate cell division. Example target regulator proteins include BBM, WUS, LEC2, GRFS, STM, E2Fa and AGL15 (SEQ ID NOs: 1-7 for example encoding nucleotide sequence and SEQ ID NOs: 8-14 for example protein sequences). The TALE DNA binding component can be configured to specifically bind the promoter sequences of the target regulator gene. For example, the TALE DNA-binding domain can be configured to selectively bind to a promoter of BBM, WUS, LEC2, GRFS, STM, E2Fa and AGL15, such as a promoter sequence with at least 90% sequence identity to one of the sequences set forth in SEQ ID NOs: 15-21. The combination of the activator agent and the promoter sequence-specific TALE DNA-binding domain facilitate the ability of the associated TALE activator to promote enhanced expression of the target regulator gene in cells that are also transformed with the non-DNA gene editing reagent. The TALE DNA binding domain and associated activator agent (e.g., the TALE activator) can be delivered in the form of an mRNA construct or a protein, so that the method and the product produced thereby remain non-transgenic and/or DNA-free.

For example, SEQ ID NOs: 1-7 can include coding sequences (CDSs) for BBM, WUS, LEC2, GRFS, STM, E2Fa and AGL15 and SEQ ID NOs: 8-14 can include the protein sequences for BBM, WUS, LEC2, GRFS, STM, E2Fa, and AGL15, which can be derived from SEQ ID NOs: 1-7 and can include protein CDSs. SEQ ID NOs: 15-21 can include nucleic acid sequences of promoters for BBM, WUS, LEC2, GRFS, STM, E2Fa and AGL15. SEQ ID NOs: 22-24 can include CDSs for the activator genes VP128, 6TAD and a 6TAD-VP128 fusion and SEQ ID NOs: 25-27 can include the protein sequences for VP128, 6TAD and a 6TAD-VP128 fusion, which can be derived from SEQ ID NOs: 22-24 and can include the protein CDSs.

As with FIG. 4, in various embodiments, the method 570 includes co-delivery of a TALEN and an in vitro purified exonuclease, such as a Trex2 mRNA or protein. Co-delivery of an exonuclease increases the efficiency of NHEJ mediated deletions at the endonuclease target cutting site, thus further increasing the likelihood/efficiency of the deletion. Further example embodiments include the triple co-delivery of the endonuclease reagent (e.g., TALEN), an exonuclease such as Trex2, and a TALE-activator, to further increase efficiency (frequency) in inducing deletions.

For convenience, certain terms employed in the specification, examples, and appended claims are provided here. The definitions are provided to aid in describing particular embodiments and are not intended to limit the claimed invention, as the scope of the invention is limited only by the claims.

The use of the term “or” in the claims and specification is used to mean “and/or” unless explicitly indicated to refer to alternatives only or the alternatives are mutually exclusive, although the disclosure supports a definition that refers to only alternatives and “and/or.”

The words “a” and “an,” when used in conjunction with the word “comprising” or “including” in the claims or specification, denotes one or more, unless specifically noted.

Unless the context clearly requires otherwise, throughout the description and the claims, the words “include”, “including”, “comprise,” “comprising,” and the like, are to be construed in an open and inclusive sense as opposed to a closed, exclusive or exhaustive sense. For example, the term “comprising” can be read to indicate “including, but not limited to.” The term “consists essentially of” or grammatical variants thereof indicate that the recited subject matter can include additional elements not recited in the claim, but which do not materially affect the basic and novel characteristics of the claimed subject matter.

Words using the singular or plural number also include the plural and singular number, respectively. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being typical. The term polypeptide or protein as used herein encompasses any amino acid sequence and includes modified sequences such as glycoproteins. The term polypeptide, unless noted otherwise, is specifically intended to cover naturally occurring proteins, as well as those that are recombinantly or synthetically produced.

One of skill will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
i. Alanine (A), Serine (S), Threonine (T),
ii. Aspartic acid (D), Glutamic acid (E),
iii. Asparagine (N), Glutamine (Q),
iv. Arginine (R), Lysine (K),
v. Isoleucine (I), Leucine (L), Methionine (M), Valine (V), and
vi. Phenylalanine (F), Tyrosine (Y), Tryptophan (W).

The term “nucleic acid” refers to a DNA or RNA nucleic acid and sequences of nucleic acids in either single- or double-stranded form, and unless otherwise limited, encompasses known analogs of natural nucleotides that hybridize to nucleic acids in manner similar to naturally occurring nucleotides. Unless otherwise indicated, a particular nucleic acid sequence includes the complementary sequence thereof.

Reference to sequence identity addresses the degree of similarity of two polymeric sequences, such as protein sequences or nucleic acid sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window can include additions or deletions (e.g., gaps) as compared to the reference sequence (which does not include additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Various embodiments are implemented in accordance with the underlying provisional application, U.S. Provisional Application No. 62/908,499, filed on Sep. 30, 2019 and entitled “DNA-Free Gene Editing”, to which benefit is claimed and is fully incorporated herein by reference. For instance, embodiments herein and/or in the provisional application may be combined in varying degrees (including wholly). Embodiments discussed in the Provisional Application are not intended, in any way, to be limiting to the overall technical disclosure, or to any part of the claimed invention unless specifically noted.

While illustrative embodiments have been illustrated and described, it will be appreciated that various changes can be made therein without departing from the scope of the invention.

### Experimental Embodiments

Various experimental embodiments were directed to designing different nucleic acid plasmid vectors, sometimes herein referred to as vectors for ease of reference and which can include the previously described nucleic acid constructs or a portion thereof, such as a DNA or mRNA construct. The vectors include a rare-cutting endonuclease and a detectable label. Specific experiments were designed to show the addition of a detectable label to the plasmid vectors, sorting of transformed protoplasts using FACS, identification and sorting of cells via a detectable label and using FACs, and genetic editing by the plasmid vectors that include the rare-cutting endonuclease and the detectable label. A number of experiments conducted are described herein.

An experiment was conducted to illustrate different nucleic acid vector designs. The different vectors are shown below in Table 1. The nucleic acid constructs in Table 1 include DNA constructs. However, as may be appreciated, the various DNA vectors can be transcribed to form an mRNA construct using the above-described in-vitro transcription techniques. The constructs include TALEN nucleic acid constructs.

TABLE 1
Constructs

| Name | Composition | Description |
|---|---|---|
| pCLS1 | NosPro-YFP-2xGGGGS- | Over expression LHT tethered with |
| | TALEN backbone | YFP, Bsal sites for TALE GG |
| | | cloning |
| pCLS2 | NosPro-RFP-2xGGGGS- | Over expression RHT tethered with |
| | TALEN backbone | RFP, Bsal sites for TALE GG |
| | | cloning |
| pCLS3 | NosPro-YFP-2xGGGGS- | Over expression LHT tethered with |
| | T03(BnFAD2)-L | YFP targeting BnFAD2 |
| pCLS4 | NosPro-RFP-2xGGGGS- | Over expression RHT tethered with |
| | T03(BnFAD2)-R | RFP targeting BnFAD2 |
| pCLS5 | T7-5'UTR-TALEN backbone | in vitro transcription LHT, Bsal |
| | L-3'UTR-PolyA | sites for TALE GG cloning |
| pCLS6 | T7-5'UTR-TALEN backbone | in vitro transcription RHT, Bsal |
| | R-3'UTR-PolyA | sites for TALE GG cloning |
| pCLS7 | T7-5'UTR-YFP-2xGGGGS- | In vitro transcription LHT tethered |
| | TALEN backbone | with YFP, Bsal sites for TALE GG |
| | L-3'UTR-PolyA | cloning |
| pCLS8 | T7-5'UTR-RFP-2xGGGGS- | In vitro transcription RHT tethered |
| | TALEN backbone | with RFP, Bsal sites for TALE GG |
| | R-3'UTR-PolyA | cloning |
| pCLS9 | T7-YFP-PolyA | In vitro transcription YFP |
| pCLS10 | T7-5'UTR-YFP-3'UTR-PolyA | In vitro transcription YFP mRNA |
| pCLS11 | T7-5'UTR-Trex2-3'UTR-PolyA | In vitro transcription TREX2 |
| | | mRNA |
| pCLS12 | T7-5'UTR-T03(BnFAD2)- | In vitro transcription LHT |
| | L-3'UTR-PolyA | targeting BnFAD2 |
| pCLS13 | T7-5'UTR-T03(BnFAD2)- | In vitro transcription RHT |
| | R-3'UTR-PolyA | targeting BnFAD2 |
| pCLS14 | T7-5'UTR-YFP-2xGGGGS- | In vitro transcription LHT tethered |
| | T03(BnFAD2)-L-3'UTR-PolyA | with YFP targeting BnFAD2 |
| pCLS15 | T7-5'UTR-RFP-2xGGGGS- | In vitro transcription RHT tethered |
| | T03(BnFAD2)-R-3'UTR-PolyA | with RFP targeting BnFAD2 |
| pCLS16 | NosPro-T03(BnFAD2)-L | Control Group: Over expression |
| | | LHT targeting BnFAD2 |
| pCLS17 | NosPro-T03(BnFAD2)-R | Control Group: Over expression |
| | | RHT targeting BnFAD2 |
| pCLS18 | T7-5'UTR-TALE-VP128- | In vitro transcription TALE-VP128 |
| | 3'UTR-PolyA | mRNA |
| pCLS19 | T7-5'UTR-TALE-6TAD- | In vitro transcription TALE-6TAD |
| | 3'UTR-PolyA | mRNA |
| pCLS20 | T7-5'UTR-TALE-6TAD- | In vitro transcription TALE-6TAD- |
| | VP128-3'UTR-PolyA | VP128 fusion mRNA |

The constructs in Table 1 that were generated in the experimental embodiments are described in detail below. The vectors pCLS3 and pCLS4 are vectors that were generated and that include a TALEN that targets the gene BnFAD2 and which are tethered to fluorescent proteins. Vector pCLS3 includes a promoter NosPro, a fluorescent protein YFP, a linker sequence 2xGGGGS, and a LHT tethered to the YFP and that targets the gene BnFAD2. Vector pCLS4 includes a promoter NosPro, a fluorescent protein RFP, a linker sequence 2xGGGGS, and a RHT tethered to the YFP and that targets the gene BnFAD2. The vectors pCLS3 and pCLS4 are complete TALEN constructs. In experimental embodiments, vectors pCLS3 and pCLS4 were used to demonstrate TALEN activity for a TALEN-Fluorescent fusion protein. Vectors pCLS14 and pCLS15 are vectors that were generated and that can be used for in-vitro transcription to generate an mRNA construct encoding a TALEN-fluorescent fusion protein. Vector pCLS14 includes a promoter T7, a 5′ UTR, a fluorescent protein YFP, a linker sequence 2xGGGGS, a LHT tethered to the YFP and that targets the gene BnFAD2, a 3′ UTR, and a poly-A tail. Vector pCLS15 includes a promoter T7, a 5′ UTR, a fluorescent protein RFP, a linker sequence 2xGGGGS, a RHT tethered to the RFP and that targets the gene BnFAD2, a 3′ UTR, and a poly-A tail. Vectors pCLS16 and pCLS17 were generated and used as controls in various experimental embodiments. Vector pCLS16 includes a promoter NosPro and a LHT that targets the gene BnFAD2. Vector pCLS17 includes a promoter NosPro and a RHT that targets the gene BnFAD2.

A full map sequence of vector pCLS3 is set forth in SEQ ID NO: 28 and an expression cassette from vector pCLS3 is set forth in SEQ ID NO: 29. A full map sequence of vector pCLS4 is set forth in SEQ ID NO: 30 and an expression cassette from vector pCLS4 is set forth in SEQ ID NO: 31. A full map sequence of vector pCLS14 is set forth in SEQ ID NO: 32 and an expression cassette from vector pCLS14 is set forth in SEQ ID NO: 33. A full map sequence of vector pCLS15 is set forth in SEQ ID NO: 34 and an expression cassette from vector pCLS15 is set forth in SEQ ID NO: 35. For example, the promoters, NosPro and T7, are based on Agrobacterium tumefaciens sequence (e.g., an Agrobacterium tumefaciens Ti plasmid), YFP is based on Aequorea victoria sequence, RFP is based on Discosoma sp sequence, and the UTRs and/or polyA tail are based on Arabidopsis thaliana sequence. The TALENs (e.g., T03(BnFAD2)-L and T03(BnFAD2)-R) are based on Brassica napus sequence, Xanthomonas sequence, and Flavobacterium okeanokoites sequence. The TALENS include a TALE effector based on Xanthomonas sequence that is further based on and targets Brassica napus sequence (e.g., targets a gene) and a Fok1 based on Xanthomonas sequence.

The remaining example constructs of Table 1 are described below. Vector pCLS1 includes a promoter NosPro, a fluorescent protein YFP, a linker sequence 2xGGGGS, and a TALEN backbone for a LHT. Vector pCLS2 includes a promoter NosPro, a fluorescent protein RFP, a linker sequence 2xGGGGS, and a TALEN backbone for a RHT. The vectors pCLS1 and pCLS2 include entry level vectors having Bsal cutting sites for TALE GG cloning. Bsal is a type II restriction endonuclease and a non-limiting example of a Bsal cutting site includes GGTCTCN′NNNN. Vectors pCLS5-pCLS13 include entry level vectors and/or portions of vectors which can be used for in-vitro transcription to generate an mRNA construct. For example, vector pCLS5 includes a promoter T7, a 5′ UTR, a TALEN backbone for a LHT, a ‘3 UTR, and a poly-A tail. Vector pCLS6 includes a promoter T7, a 5’ UTR, a TALEN backbone for a RHT, a ‘3 UTR, and a poly-A tail. Vector pCLS7 includes a promoter T7, a 5’ UTR, a fluorescent protein YFP, a linker sequence 2xGGGGS, a TALEN backbone for a LHT, a ‘3 UTR, and a poly-A tail. Vector pCLS8 includes a promoter T7, a 5’ UTR, a fluorescent protein RFP, a linker sequence 2xGGGGS, a TALEN backbone for a RHT, a ‘3 UTR, and a poly-A tail. Vector pCLS9 includes a promoter T7, a fluorescent protein YFP, and a poly-A tail. Vector pCLS10 includes a promoter T7, a 5’ UTR, a fluorescent protein YFP, a ‘3 UTR, and a poly-A tail. Vector pCLS11 includes a promoter T7, a 5’ UTR, Trex2, a ‘3 UTR, and a poly-A tail. Vector pCLS12 includes a promoter T7, a 5’ UTR, a LHT that targets the gene BnFAD2, a 3′ UTR, and a poly-A tail. Vector pCLS13 includes a promoter T7, a 5′ UTR, a RHT that targets the gene BnFAD2, a 3′ UTR, and a poly-A tail. Embodiments are not limited to targeting of a specific gene, such as BnFAD2.

Various embodiments are directed to constructs that include activator agents, such as illustrated by vectors pCLS18- pCLS20. For example, vector pCLS18 includes a promoter T7, a ‘5 UTR, a TALEN, an activator agent VP128, a 3’ UTR, and a poly-A tail. Vector pCLS19 includes a promoter T7, a ‘5 UTR, a TALEN, an activator agent 6TAD, a 3’ UTR, and a poly-A tail. Vector pCLS20 includes a promoter T7, a ‘5 UTR, a TALEN, a first activator agent VP128, a second activator agent 6TAD, a 3’ UTR, and a poly-A tail.

Another example experiment was conducted to illustrate transformation of protoplasts with detectable labels. More specifically, canola protoplasts were transformed using the nucleic acid constructs illustrated in Table 2. As shown in Table 2, the constructs were DNA constructs that encoded fluorescent proteins used to label the canola protoplast. Table 3 illustrates example results of sorting the transformed canola protoplasts by the florescent proteins using FACS.

TABLE 2
DNA vectors

| | Plasmid | | | | Conc. | Vol. | Protopl. |
|---|---|---|---|---|---|---|---|
| Sample | Vector | Description | Type | Quant. | (ng/ul) | (ul) | # |
| 1 | neg ctrl | | DNA | 0 | 0 | 0 | 200K |
| 2 | pCLS21 | VaUBI3_YFP_nosT | DNA | 30 ul | 2329 | 12.88 | 200K |
| 3 | pCLS22 | MtEF1A_YFP_nosT | DNA | 30 ul | 5726 | 5.24 | 200K |
| 4 | pCLS23 | CaMV35S_RFP_nosT | DNA | 30 ul | 3738 | 8.02 | 200K |
| 5 | pCLS21 | YFP & RFP | DNA | 30 ug | | 12.88 | 200K |
| | & pCLS23 | | | each | | 8.02 | |
| 6 | neg ctrl | | DNA | 0 | | 0 | 200K |
| 7 | pCLS21 | VaUBI3_YFP_nosT | DNA | 30 ug | | 12.88 | 200K |
| 8 | pCLS22 | MtEF1A_YFP_nosT | DNA | 30 ug | | 5.24 | 200K |
| 9 | pCLS23 | CaMV35S_RFP_nosT | DNA | 30 ug | | 8.02 | 200K |
| 10 | pCLS21 | YFP & RFP | DNA | 30 ug | | 12.88 | 200K |
| | & pCLS23 | | | each | | 8.02 | |

TABLE 3
FACS canola protoplasts with fluorescent
protein expression DNA vector

| | | Total | | |
|---|---|---|---|---|
| | | positive | Processed | Positive |
| Sample | Label | cells | cells | ratio (%) |
| Sample 2 | YFP | 3939 | 39628 | 9.939941456 |
| Sample 4 | RFP | 5763 | 70048 | 8.227215624 |
| Sample 5 | YFP & RFP | 12221 | 66907 | 18.26565232 |

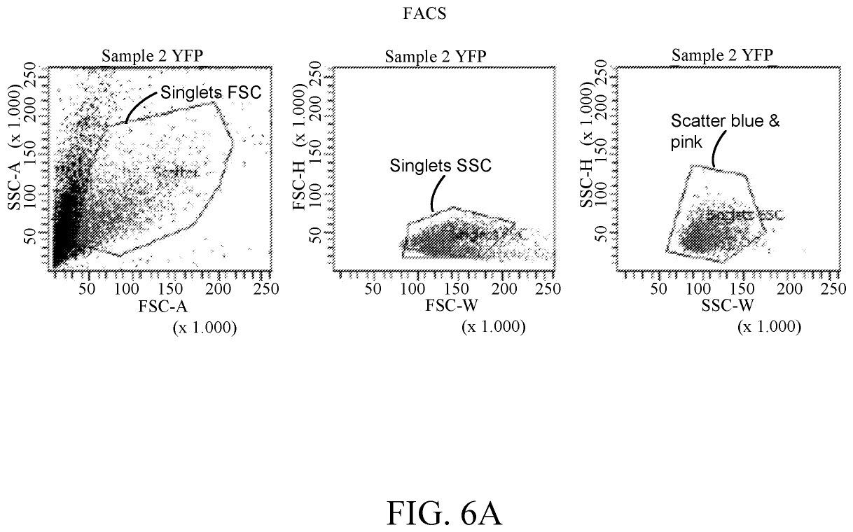

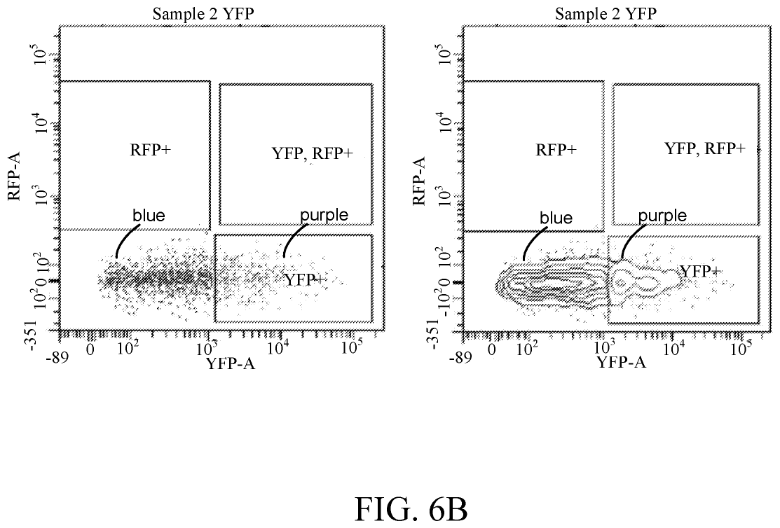

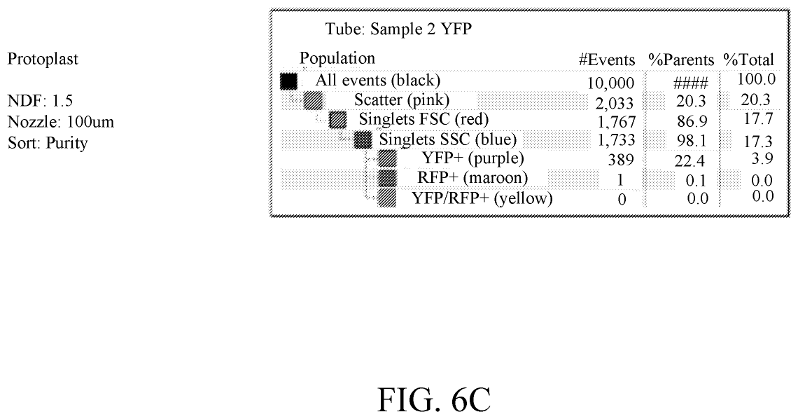

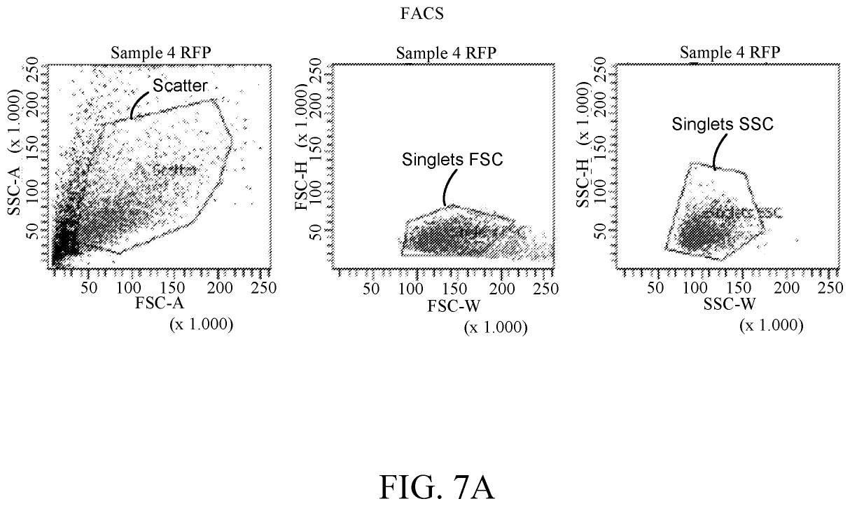

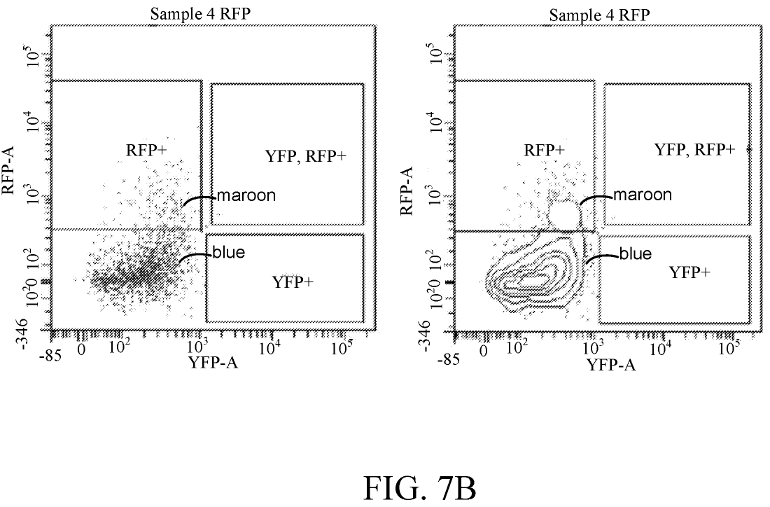

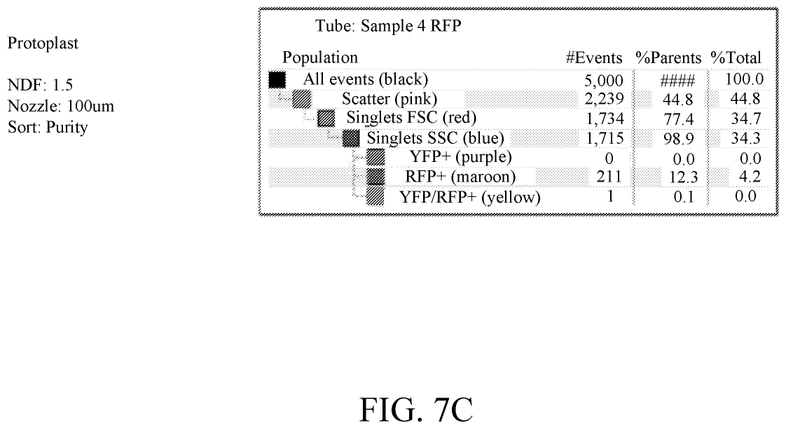

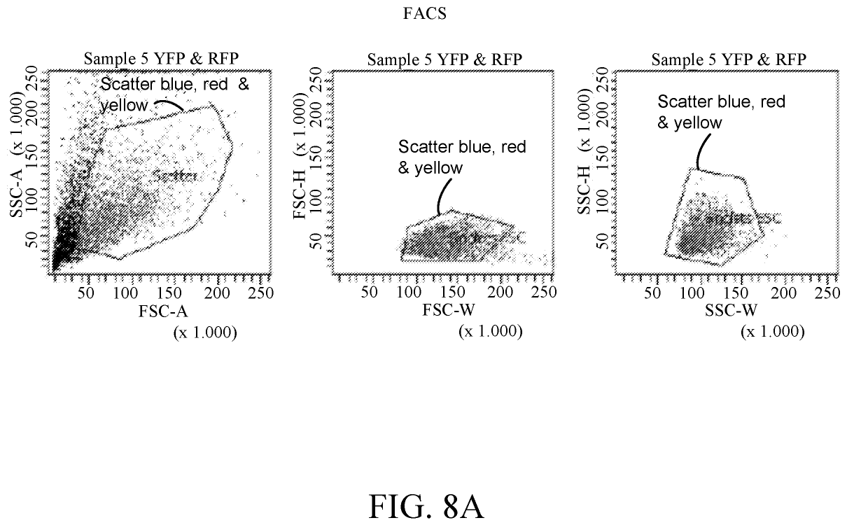

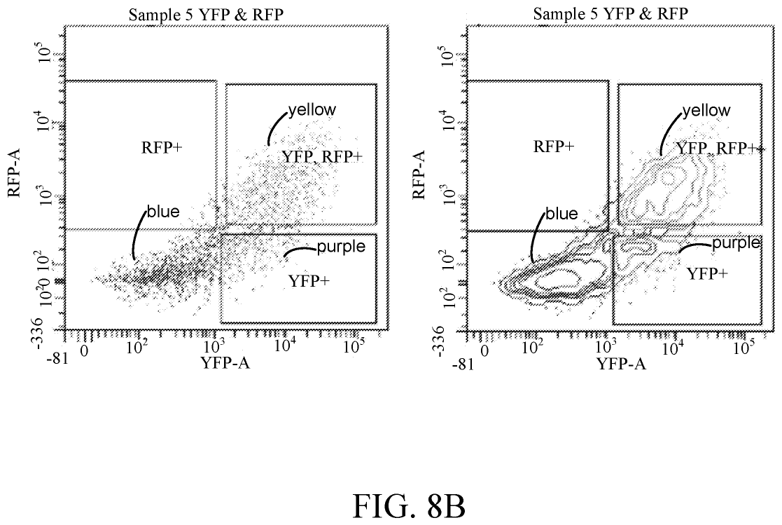

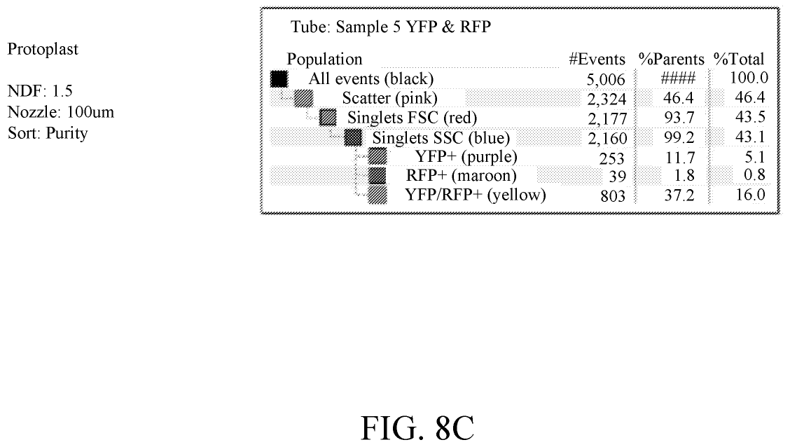

FIGS. 6A-8C illustrate example flow cytometry data demonstrating sorting of protoplasts transformed using the nucleic acid constructs of Table 2, consistent with the present disclosure. For example, FIGS. 6A-8C show raw data from flow cytometry experiments demonstrating the ability to sort plant protoplasts using fluorescence. FIGS. 6A-6C show raw flow cytometry data from experimental results of sorting Sample 2 in Table 2 which included canola protoplasts transformed to express YFP using vector pCLS21. FIGS. 7A-7C show raw flow cytometry data from experimental results of sorting Sample 4 in Table 2 which included canola protoplasts transformed to express RFP using vector pCLS23. FIGS. 8A-8C show raw flow cytometry data from experimental results of sorting Sample 5 in Table 2 which included canola protoplasts transformed to express YFP and RFP using vectors pCLS21 and pCLS23.

A further example experiment was conducted to show protoplast transformed with a nucleic acid construct that has a rare-cutting endonuclease and a detectable label. For example, canola protoplasts were transformed using plasmid vectors illustrated by Table 4.

TABLE 4
Canola Protoplasts Transformation

| | Plasmid | | Conc | Vol | Plasmid | | Conc | Vol |
|---|---|---|---|---|---|---|---|---|
| Sample | 1 | Descrip | (ng/ul) | (ul) | 2 | Descrip | (ng/ul) | (ul) |
| A | pCLS3 | NosPro-YFP-2xGGGGS- | 3897 | 5.2 | pCLS4 | NosPro-RFP-2xGGGGS- | 2498 | 8 |
| | | T03(BnFAD2)- | | | | T03(BnFAD2)-R | | |
| | | LBnFAD2_T03-L1 | | | | | | |
| B | pCLS16 | BnFAD2_T03-L1 | 6072 | 3.3 | pCLS17 | BnFAD2_T03-R1 | 4130 | 4.8 |
| C | pCLS3 | NosPro-YFP-2xGGGGS- | 3897 | 5.2 | pCLS4 | NosPro-RFP-2xGGGGS- | 2498 | 8 |
| | | T03(BnFAD2)-L | | | | T03(BnFAD2)-R | | |
| D | pCLS16 | BnFAD2_T03-L1 | 6072 | 3.3 | pCLS17 | BnFAD2_T03-R1 | 4130 | 4.8 |
| E | pCLS21 | VaUBI3_YFP_nosT | | | pCLS23 | CaMV35S_RFP_nosT | | |
| F | pCLS21 | VaUBI3_YFP_nosT | | | | | | |

Table 4 illustrates example nucleic acid constructs used to transform canola protoplasts. The constructs generated included previously described vectors pCLS3, pCLS4, pCLS16, pCLS17, p pCLS21, and pCLS23. Each of the plasmid vectors 1 and 2 (e.g., referred to as “Plasmid 1” and “Plasmid 2”) of Samples A-F included DNA and a quantity of 20 ug. Samples A-E of Table 4 included a 200,000 protoplasts. Samples A-D were prepared using the same Illumina sequence for analysis. Samples E-F were used as controls. The vectors were used to transform canola protoplasts to compare the gene editing efficiency of fluorescently labeled TALEN nucleic acid constructs as compared to constructs without fluorescent labels. As described above, vectors pCLS3 and pCLS4 included the fluorescent proteins YFP and RFP, and vectors pCLS16 and pCLS17 did not. Vectors pCLS21 and pCLS23 were used as controls and included fluorescent labels.

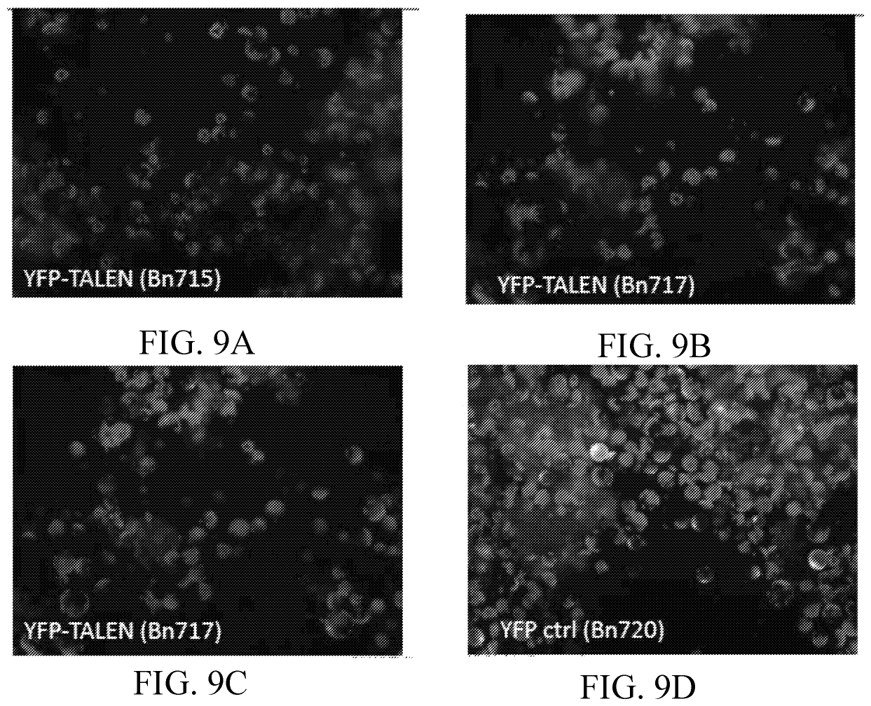

FIGS. 9A-9D illustrate microscopy images of plant cells transformed using the nucleic acid constructs of Table 4, consistent with the present disclosure. FIG. 9A illustrates a microscopy image of canola plant cells from Sample A of Table 4. Sample A included canola protoplasts transformed using vectors pCLS3 and pCLS4. FIGS. 9B-9C illustrate microscopy images of canola plant cells from Sample C of Table 4. Sample C included canola protoplasts transformed using vectors pCLS3 and pCLS4. FIG. 9D illustrates a microscopy image of canola plant cells from Sample F of Table 4. Sample F included a control group of canola protoplasts transformed using vector pCLS21. The images of FIGS. 9A-9C demonstrate expression of YFP-TALEN fusion protein located in the nucleus of protoplasts.

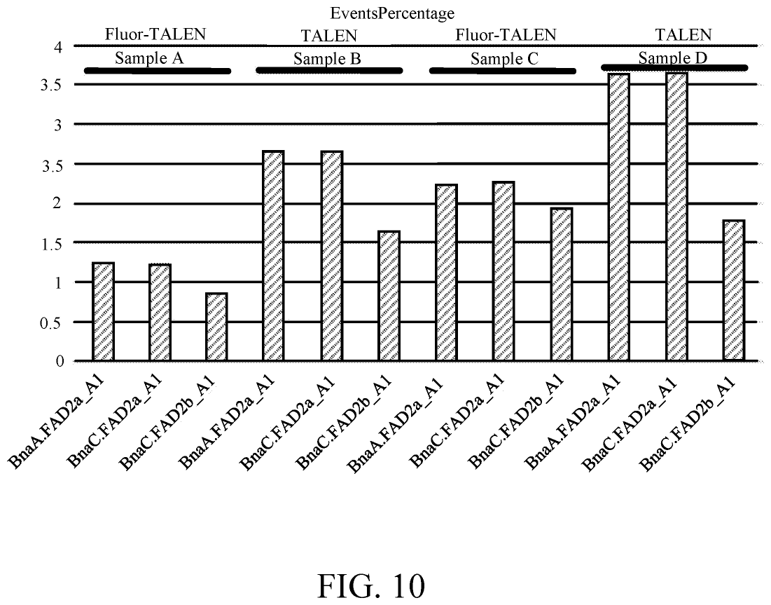

FIG. 10 illustrates detected deletions of plants transformed using the nucleic acid constructs of Table 4, consistent with the present disclosure. The gene editing efficiencies of Samples A, B, C, and D from Table 4 were compared. Samples A and C included canola protoplasts transformed with constructs encoding TALENs fused to florescent protein (e.g., fusion proteins). Samples B and D included canola protoplasts transformed with constructs encoding TALENs without a detectable label. The TALENs in all Samples A-D targeted the gene BnFAD2. The graph of FIG. 10 illustrates results of a NHEJ mutation assay used to detect deletions in a population of protoplast cells that were transformed with the TALEN or Fluor-TALEN vector plasmids. As shown, Samples A and C resulted in detected deletions representative of activity of TALENS without detectable labels, such as Samples B and D.

The above described experimental embodiments demonstrate detectable labels being expressed by protoplasts, successfully sorting protoplasts expressing the detectable labels via FACS, and TALEN activity resulting from protoplasts expressing the detectable labels. Embodiments in accordance with the present disclosure are not limited to that demonstrated by the experimental embodiments and can include a variety of different types of constructs including different types of endonucleases, detectable labels, target genes, and mutations.

### SEQUENCE LISTING FREE TEXT

SEQ ID NOs: 1-21 are each based on Glycine max sequence. SEQ ID NOs: 22 and 25 are each based on herpes simplex virus sequence. SEQ ID NOs: 23 and 26 are each on based on Xanthomonas campestris sequence. SEQ ID NOs: 24 and 27 are each based on herpes simplex virus sequence and Xanthomonas campestris sequence. SEQ ID NOs: 28 and 29 are each a synthetic construct based on Agrobacterium tumefaciens sequence, Aequorea victoria sequence, Brassica napus sequence, Xanthomonas sequence, and Flavobacterium okeanokoites sequence. SEQ ID NOs: 30 and 31 are each a synthetic construct based on Agrobacterium tumefaciens sequence, Discosoma sp sequence, Brassica napus sequence, Xanthomonas sequence, and Flavobacterium okeanokoites sequence. SEQ ID NOs: 32 and 33 are each a synthetic construct based on Agrobacterium tumefaciens sequence, Aequorea victoria sequence, Arabidopsis thaliana sequence, Xanthomonas sequence, and Flavobacterium okeanokoites sequence. SEQ ID NOs: 34 and 35 are each a synthetic construct based on Agrobacterium tumefaciens sequence, Discosoma sp sequence, Arabidopsis thaliana sequence, Xanthomonas sequence, and Flavobacterium okeanokoites sequence.

### SEQUENCE LISTING

(GmBBM1 CDS)
SEQ ID NO: 1
ATGGGGTCTATGAATTTGTTAGGTTTTTCTCTCTCTCCTCACGAAGAACA
CCCTTCTAGTCAAGATCACTCTCAAACGACACCTTCTCGTTTTAGCTTCA
ACCCTGATGGATCAATCTCAAGCACTGATGTAGCAGGAGGCTGCTTTGAT
CTCACTTCTGACTCAACTCCTCATTTACTTAACCTTCCTTCTTATGGCAT
ATACGAAGCATTTCACAGAAACAATAGTATTAACACCACTCAAGATTGGA
AGGAGAACTACAACAGCCAAAATTTGCTATTGGGAACTTCGTGCAATAAA
CAAAACATGAACCAAAACCAACAGCAACAGCCAAAGCTTGAAAACTTCCT
CGGTGGACACTCATTTGGCGAACATGAGCAAACCTACGGTGGTAACTCAG
CCTCTACAGATTACATGTTTCCTGCTCAGCCAGTATCGGCTGGTGGTGGT
GGTAGTGGTGGTGGCAGTAACAATAACAACAACAGTAACTCCATAGGGTT
ATCCATGATAAAGACATGGTTGAGGAACCAACCACCGAACTCAGAAAACA
TCAACAACAACAATGAAAGTGGTGGCAATATTAGAAGCAGTGTGCAGCAA
ACTCTATCACTTTCCATGAGTACTGGTTCACAATCAAGCACATCACTGCC
CCTTCTCACTGCTAGTGTGGATAATGGAGAGAGTTCTTCTGATAACAAAC
AACCAAACACCTCGGCTGCACTTGATTCCACCCAAACCGGAGCCATTGAA
ACTGCACCCAGAAAGTCCATTGACACTTTTGGACAGAGAACTTCTATCTA
CCGTGGTGTAACAAGGCATAGGTGGACGGGGAGGTACGAGGCTCACCTGT
GGGATAATAGTTGTAGAAGAGAGGGACAGACTCGCAAAGGAAGGCAAGGT
GGTTATGATAAAGAAGAAAAGGCAGCTAGAGCCTACGATTTGGCAGCACT
AAAATACTGGGGAACAACCACAACAACAAATTTTCCAATTAGCCACTATG
AGAAAGAGTTGGAAGAAATGAAGCACATGACTAGGCAAGAGTACGTTGCG
TCATTGAGAAGGAAGAGTAGTGGGTTTTCTCGCGGTGCATCCATTTATCG
AGGAGTGACGAGACACCACCAACATGGAAGGTGGCAAGCGAGGATTGGAA
GAGTTGCTGGCAACAAGGATCTTTACTTGGGAACTTTTAGCACCCAAGAA
GAGGCAGCGGAAGCATATGATGTAGCAGCAATCAAATTCCGAGGACTAAG
TGCTGTTACAAACTTTGACATGAGCAGATATGACGTGAAAAGCATACTTG
AGAGCACCACTTTGCCAATAGGTGGTGCTGCAAAGCGTTTGAAGGATATG
GAGCAGGTTGAACTGAGTGTGGATAATGGTCATAGAGCAGATCAAGTAGA
TCATAGTATCATCATGAGTTCTCACCTAACTCAAGGAATCAATAACAACT
ATGCAGGAGGGGGAACAGCAACTCATCATAACTGGCACAATGCTCATGCA
TTCCACCAACCTCAACCTTGCACCACCATGCACTACCCTTATGGACAAAG
AATTAATTGGTGCAAGCAAGAACAACAAGACAACTCTGATGCCCCTCACT
CTTTGTCTTATTCAGATATTCATCAACTTCAGCTAGGGAACAATGGAACA
CATAACTTCTTTCACACAAATTCAGGGTTGCACCCTATGTTGAGCATGGA
TTCTGCTTCCATTGACAATAGCTCTTCTTCTAACTCGGTTGTTTATGATG
GTTATGGAGGTGGTGGGGGCTACAATGTGATGCCTATGGGAACTACTACT
GCTGTTGTTGCAAGTGATGGTGATCAAAATCCAAGAAGCAATCATGGTTT
TGGTGATAATGAGATAAAAGCACTTGGTTATGAAAGTGTGTATGGCTCTG
CAACTGATTCTTATCATGCACATGCAAGGAACTTGTATTATCTTACTCAA
CAGCAATCATCTTCTGTTGATACAGTGAAGGCTAGTGCATATGATCAAGG
GTCTGCATGCAATACTTGGGTTCCAACTGCTATTCCAACTCATGCACCCA
GATCAACTACTAGTATGGCTCTCTGCCATGGGGCTACTACACCCTTCTCT
TTATTGCATGAATAG
(GmWUS CDS)
SEQ ID NO: 2
ATGATGGAACCTCAACAACAACAACAACAAGCACAAGGGAGCCAACAACA
ACAACAAAACGAGGATGGTGGCAGTGGAAAAGGGGGGTTTCTGAGCAGGC
AAAGTAGTACACGGTGGACTCCAACAAACGACCAGATAAGAATATTGAAG
GAACTTTACTACAACAATGGAATTAGATCCCCGAGTGCAGAGCAGATTCA
GAGGATCTCTGCTAGGCTGAGGCAGTACGGTAAGATTGAAGGCAAGAATG
TCTTTTATTGGTTCCAGAACCACAAAGCTCGAGAAAGGCAGAAGAAAAGG
TTCACTTCTGATCATAATCATAATAATGTCCCCATGCAAAGACCCCCAAC
TAATCCTTCTGCTGCTTGGAAACCTGATCTAGCTGATCCCATTCACACCA
CCAAGTATTGTAACATCTCTTCTACTGCAGGGATCTCTTCGGCATCATCT
TCTGTTGAGATGGTTACTGTGGGACAGATGGGGAATTATGGGTATGGTTC
TGTGCCCATGGAGAAAAGTTTTAGGGACTGCTCGATATCAGCTGGGGGTA
GCAGTGGCCATGTTGGATTAATAAACCACAACTTGGGGTGGGTTGGTGTG
GACCCATATAATTCCTCAACCTATGCCAACTTCTTTGACAAAATAAGGCC
AAGTGATCAAGAAACCCTTGAAGAAGAAGCAGAGAACATTGGTGCTACTA
AGATTGAAACCCTCCCTTTATTCCCTATGCACGGTGAGGACATCCATGGC
TATTGCAACCTCAAGTCTAATTCGTATAACTATGATGGAAACGGCTGGTA
TCATACTGAAGAAGGGTTCAAGAATGCTTCTCGTGCTTCCTTGGAGCTCA
GTCTCAACTCCTACACTCGCAGGTCTCCAGATTATGCTTAA
(GmLEC2 CDS)
SEQ ID NO: 3
ATGGAAAACTTTTTTGTGCCATTTTTAAAAAAAAACCCCAACCCATCAAT
CACCACTACTGGTGGCAATGGCTCATCTTCATCAAACCAAACAAGCCTTG
TACAACCAAGCACATATCCTCAAAATTTCCCTTACAATACTAGTGTAAAA
CTTAACTTTCCAGAACAACCTTATTTCATTCCTTTGTATCCCTTTCCAAC
AGGACAAGTTAGCTTTTCTAATCAACCCTATGGAATGCCAAATTCGGAAC
TTCAAGGTTCGAGGGCATGCATGACCAAAGCTACAAGGGAGAGATGGAGA
CAAGTAAGACAAAGGAGTAAAAATTCTACTCTTGTCGCTCCTAATTCAGT
TCTAGAAAGGACAACAAGAGAACAATTTGTTCCTAATGGAGGGTCAAATG
TGAGGATCACAGTCAAACAACACAATGCAACCAAGTTTTTTAACACCCCA
AACGGGAAGAAGCTAGAAGAAATTTTGACAAAGAAGTTGAATAATAGTGA
TGTTGGCGTCCTAGGCCGCATTGTGCTCCCAAAGAGAGAGGCTGAGGATA
AGCTTCCGACACTGTGGAAGAAGGAAGGAATCAATATTGTACTAAAGGAT
GTATATTCTGAGATTGAATGGAGCATCAAATACAAGTACTGGACTAATAA
CAAAAGCAGAATGTATATTCTTGATAATACAGGGGATTTTGTTAACCATT
ATAAACTTCAAACAGGAGATTTCATAACCCTTTACAAGGACGAGTTGAAA
AATCTGTATGTGTCGGCTCGAAAGGATCAAGAAAATCTAGAAGAATCTAA
GTCCTCGTCAAACACAGGAATGTCACATGAACCAGATGCATATTTAGCTT
ACTTGACGAAGGAACTTAGCCATAAGGGGAAAGCAGAAGCTGCCAACAAC
CTTTTGAACAATGTTGAGGAAGAGGCACCAAATCAAGCAAATCAATTACA
TCAATTCATGCCGATGAACAATATTGTTGGGGAGGGGGCATCAAACCAAG
CAATTCAAGAAGCCGCACCAGCCGCACCCGTCAATGTTAATCAAGAAAAC
AAAGTTGTTGACGACGATGATGATGATATCTATGGTGGCCTTGACAATAT
TTTCGAAATTGGAAATACTTATCAAATTTGGTAG
(GmGRF5 CDS)
SEQ ID NO: 4
ATGATGAGTGCAAGTGCAAGAAATAGGTCTCCTTTCACGCAAACTCAGTG
GCAAGAGCTTGAGCATCAAGCTCTTGTTTTTAAGTACATGGTTACAGGAA
CACCCATCCCACCAGATCTCATCTACTCTATTAAAAGAAGTCTAGACACT
TCAATTTCTTCAAGGCTCTTCCCACATCATCCAATTGGGTGGGGATGTTT
TGAAATGGGATTTGGCAGAAAAGTAGACCCAGAGCCAGGGAGGTGCAGAA
GAACAGATGGCAAGAAATGGAGATGCTCAAAGGAGGCATATCCAGACTCC
AAGTACTGTGAAAGACACATGCACAGAGGCAGAAACCGTTCAAGAAAGCC
TGTGGAAGTTTCTTCAGCAATAAGCACCGCCACAAACACCTCCCAAACAA
TCCCATCTTCTTATACCCGAAACCTTTCCTTGACCAACCCCAACATGACA
CCACCCTCTTCCTTCCCTTTCTCTCCTTTGCCCTCTTCTATGCCTATTGA
GTCCCAACCCTTTTCCCAATCCTACCAAAACTCTTCTCTCAATCCCTTCT
TCTACTCCCAATCAACCTCCTCTAGACCCCCAGATGCTGATTTTCCACCC
CAAGATGCCACCACCCACCAGCTATTCATGGACTCTGGGTCTTATTCGCA
TGATGAAAAGAATTATAGGCATGTTCATGGAATAAGAGAAGATGTGGATG
AGAGAGCTTTCTTCCCAGAAGCATCAGGATCAGCTAGGAGCTACACTGAA
TCATACCAGCAACTATCAATGAGCTCCTACAAGTCCTATTCAAACTCCAA
CTTTCAGAACATCAATGATGCCACCACCAACCCAAGACAGCAAGAGCAGC
AACAACAACAACACTGCTTTGTTTTGGGGACAGACTTCAAATCAACAAGA
CCAACTAAAGAGAAAGAAGCTGAGACAGCTACGGGTCAGAGACCCCTTCA
CCGTTTCTTTGGGGAGTGGCCACCAAAGAACACAACAGATTCATGGCTAG
ATCTTGCTTCCAACTCCAGAATCCAAACCGATGAATGA
(GmSTM CDS)
SEQ ID NO: 5
ATGGAGGGTAGTAGTTGCTCTAATGACACTTCTTATTTGTTGGCTTTTGG
AGAAAACAGTGGTGGGCTATGCCCAATGACGATGATGCCTTTGGTAACTT
CCCATCATGCAACAAATCCTAGTAATCCTAGTAATAATACTAATAATAAT
GAAAACACAAACTGTCTCTTCATTCCCAACTGCAGTAACAGTTCTGGAAC
TCCTTCTATCATGCTCCACAACAACAACAACACTGATGATGATAACAACA
AAACCAGCACTAACACTGGGTTAGGGTACTATTTCATGGAGAGTGACCAC
CATCACCGCAACAACAACAACAATGGAAGCTCCTCCTCCTCTTCCTCTTC
TGCTGTCAAGGCCAAGATCATGGCTCATCCTCACTATCACCGTCTCTTGG
CAGCTTACGTCAATTGTCAGAAGGTTGGAGCCCCACCGGAAGTGGTGGCA
AGGTTAGAAGAAGCATGTGCTTCTGCAGCGACAATGGCTGGTGATGCAGC
AGCAGCAGCTGGATCAAGCTGCATAGGTGAAGATCCAGCTTTGGATCAGT
TCATGGAGGCTTACTGTGAGATGCTCACCAAGTATGAGCAAGAACTCTCC
AAACCCTTAAAGGAAGCCATGCTCTTCCTTCAAAGGATTGAGTGCCAGTT
CAAAAATCTTACAATTTCTTCCACCGACTTTGCTTGCAACGAGGGTGCTG
AGAGGAATGGATCATCTGAAGAGGATGTTGATCTACACAACATGATAGAT
CCCCAGGCAGAGGACAGGGAATTAAAGGGTCAGCTTTTGCGCAAGTACAG
CGGATACCTGGGCAGTCTGAAGCAAGAATTCATGAAGAAGAGGAAGAAAG
GAAAGCTACCTAAAGAAGCAAGGCAACAATTACTTGAATGGTGGAGCAGA
CATTACAAATGGCCTTACCCATCCGAGTCACAGAAGCTGGCCCTTGCAGA
GTCGACAGGTCTGGATCAGAAGCAAATCAACAACTGGTTTATTAATCAAA
GGAAACGGCACTGGAAGCCTTCAGAGGACATGCAGTTTGTGGTGATGGAT
CCAAGCCATCCACACTATTACATGGATAATGTTCTGGGCAATCCATTTCC
CATGGATCTCTCCCATCCAATGCTCTAG
(GmE2FA CDS)
SEQ ID NO: 6
ATGTCCAGCGCCGCCGGAGTTCCCGACCGCCTCGCTTCGCAGCCGCGGGG
GGCTGCCGGCGCCCCTGCCCTCCCGCCGCTCAAGCGCCACCTTGCCTTCG
TCACGAAACCGCCCTTCGCCCCGCCCGATGAGTACCACAGCTTCTCCAGT
GCCGACTCCCGCCGCGCCGCGGATGAAGCCGTCGTCGTTAGATCTCCGTA
CATGAAGCGGAAGAGTGGAATGACTGACAGTGAAGGGGAGTCACAAGCAC
AAAAGTGGAGTAACAGCCCAGGATACACTAATGTTAGTAATGTAACGAAT
AATAGTCCCTTCAAAACTCCTGTGTCTGCAAAAGGGGGAAGGGCACAGAA
GGCAAAGGCTTCCAAAGAAGGCAGATCATGTCCTCCGACACCCATGTCAA
ATGCTGGTTCCCCTTCTCCTCTTACTCCTGCTAGCAGCTGTCGCTATGAC
AGTTCCTTAGGTCTCTTGACAAAAAAGTTCATCAATTTGGTCAAACATGC
GGAGGATGGTATTCTTGACCTAAATAAAGCAGCAGAAACTTTGGAGGTGC
AAAAGAGGAGGATATATGACATAACTAATGTTTTGGAAGGCATTGGTCTC
ATTGAAAAGAAGCTCAAGAACAGAATACATTGGAAGGGAATTGAATCTTC
TACGTCTGGTGAGGTGGATGGTGATATCTCTGTGCTTAAGGCAGAAGTTG
AGAAACTTTCTTTGGAGGAGCAGGGATTAGATGATCAAATAAGGGAAATG
CAAGAAAGGCTGAGGAATTTGAGTGAAAATGAAAACAACCAGAAGTGCCT
TTTCGTGACTGAAGAAGATATTAAGGGCCTGCCTTGCTTCCAGAATGAAA
CTTTAATAGCAATTAAAGCTCCGCATGGAACCACCCTGGAAGTCCCTGAT
CCTGAGGAAGCTGTAGACTATCCGCAGAGAAGATATAGAATCATTCTTAG
AAGCACAATGGGCCCCATTGATGTCTACCTTATCAGTCAATTTGAAGAGA
AATTTGAAGAGGTTAATGGTGCTGAGCTCCCCATGATCCCACTTGCTTCC
AGTTCTGGTTCCAATGAGCAACTAATGACGGAAATGGTTCCTGCTGAATG
CAGCGGAAAAGAACTTGAACCTCAAACTCAGCTCTCTTCTCATGCATTCT
CTGATCTAAATGCTTCACAGGAGTTTGCTGGTGGCATGATGAAGATTGTC
CCTTCAGATGTTGATAATGATGCAGATTATTGGCTTCTATCAGATGCTGA
CGTTAGTATAACAGATATGTGGAGAACAGATTCTACTGTTGATTGGAATG
GTATAGACATGCTTCATCCTGATTTTGGAATCATTTCGAGGCCTCAAAGT
CCATCATCTGGGCTTGCTGAAGTGCCATCAACAGGAGCAAACTCTATTCA
GAAGTGA
(GmAGL15 CDS)
SEQ ID NO: 7
ATGGGTCGAGGGAAAATCGAGATCAAAAGAATCGACAATGCTAGCAGCAG
ACAAGTCACGTTCTCGAAGCGGAGAACAGGGTTGTTCAAGAAGGCTCAGG
AACTTTCCATTCTCTGTGACGCCGAGGTTGCTGTCATAGTTTTCTCCAAC
ACTGGCAAGCTCTTCGAGTTTTCCAGTTCCGGTATGAAGCGAACACTTTC
AAGATACAACAAATGCCTTGGTTCTACAGATGCTGCTGTAGCAGAAATTA
TGACACAGAAGGAAGATTCTAAGATGGTGGAGATTCTAAGAGAGGAAATT
GAAAAGCTAGAAACAAAGCAATTACAGTTGGTGGGTAAGGATCTGACAGG
ATTGGGTTTAAAGGAATTGCAAAATTTAGAGCAGCAACTTAATGAGGGGT
TATTGTCTGTCAAGGCGAGAAAGGAGGAATTACTCATGGAGCAACTAGAG
CAATCTAGAGTTCAGGAACAGCGGGTTATGTTGGAGAATGAAACTTTGCG
AAGACAGATTGAGGAGCTTCGGTGTCTGTTTCCACAATCAGAAAGCATGG
TCCCATTCCAATACCAACATACTGAAAGAAAGAATACTTTTGTAAATACT
GGCGCCAGATGTCTCAACTTGGCTAATAACTGTGGAAATGAGAAAGGGAG
TTCAGATACAGCATTTCATTTGGGGTTGCCTGCTGGTGTTCAAGAGGAAG
GCCCCCAAGAAAGAAACCTTTTCAAATGA
(GmBBM1 Protein)
SEQ ID NO: 8
MGSMNLLGFSLSPHEEHPSSQDHSQTTPSRFSFNPDGSISSTDVAGGCFD
LTSDSTPHLLNLPSYGIYEAFHRNNSINTTQDWKENYNSQNLLLGTSCNK
QNMNQNQQQQPKLENFLGGHSFGEHEQTYGGNSASTDYMFPAQPVSAGGG
GSGGGSNNNNNSNSIGLSMIKTWLRNQPPNSENINNNNESGGNIRSSVQQ
TLSLSMSTGSQSSTSLPLLTASVDNGESSSDNKQPNTSAALDSTQTGAIE
TAPRKSIDTFGQRTSIYRGVTRHRWTGRYEAHLWDNSCRREGQTRKGRQG
GYDKEEKAARAYDLAALKYWGTTTTTNFPISHYEKELEEMKHMTRQEYVA
SLRRKSSGFSRGASIYRGVTRHHQHGRWQARIGRVAGNKDLYLGTFSTQE
EAAEAYDVAAIKFRGLSAVTNFDMSRYDVKSILESTTLPIGGAAKRLKDM
EQVELSVDNGHRADQVDHSIIMSSHLTQGINNNYAGGGTATHHNWHNAHA
FHQPQPCTTMHYPYGQRINWCKQEQQDNSDAPHSLSYSDIHQLQLGNNGT
HNFFHTNSGLHPMLSMDSASIDNSSSSNSVVYDGYGGGGGYNVMPMGTTT
AVVASDGDQNPRSNHGFGDNEIKALGYESVYGSATDSYHAHARNLYYLTQ
QQSSSVDTVKASAYDQGSACNTWVPTAIPTHAPRSTTSMALCHGATTPFS
LLHE
(GmWUS Protein)
SEQ ID NO: 9
MMEPQQQQQQAQGSQQQQQNEDGGSGKGGFLSRQSSTRWTPTNDQIRILK
ELYYNNGIRSPSAEQIQRISARLRQYGKIEGKNVFYWFQNHKARERQKKR
FTSDHNHNNVPMQRPPTNPSAAWKPDLADPIHTTKYCNISSTAGISSASS
SVEMVTVGQMGNYGYGSVPMEKSFRDCSISAGGSSGHVGLINHNLGWVGV
DPYNSSTYANFFDKIRPSDQETLEEEAENIGATKIETLPLFPMHGEDIHG
YCNLKSNSYNYDGNGWYHTEEGFKNASRASLELSLNSYTRRSPDYA
(GmLEC2 Protein)
SEQ ID NO: 10
MENFFVPFLKKNPNPSITTTGGNGSSSSNQTSLVQPSTYPQNFPYNTSVK
LNFPEQPYFIPLYPFPTGQVSFSNQPYGMPNSELQGSRACMTKATRERWR
QVRQRSKNSTLVAPNSVLERTTREQFVPNGGSNVRITVKQHNATKFFNTP
NGKKLEEILTKKLNNSDVGVLGRIVLPKREAEDKLPTLWKKEGINIVLKD
VYSEIEWSIKYKYWTNNKSRMYILDNTGDFVNHYKLQTGDFITLYKDELK
NLYVSARKDQENLEESKSSSNTGMSHEPDAYLAYLTKELSHKGKAEAANN
LLNNVEEEAPNQANQLHQFMPMNNIVGEGASNQAIQEAAPAAPVNVNQEN
KVVDDDDDDIYGGLDNIFEIGNTYQIW
(GmGRF5 Protein)
SEQ ID NO: 11
MMSASARNRSPFTQTQWQELEHQALVFKYMVTGTPIPPDLIYSIKRSLDT
SISSRLFPHHPIGWGCFEMGFGRKVDPEPGRCRRTDGKKWRCSKEAYPDS
KYCERHMHRGRNRSRKPVEVSSAISTATNTSQTIPSSYTRNLSLTNPNMT
PPSSFPFSPLPSSMPIESQPFSQSYQNSSLNPFFYSQSTSSRPPDADFPP
QDATTHQLFMDSGSYSHDEKNYRHVHGIREDVDERAFFPEASGSARSYTE
SYQQLSMSSYKSYSNSNFQNINDATTNPRQQEQQQQQHCFVLGTDFKSTR
PTKEKEAETATGQRPLHRFFGEWPPKNTTDSWLDLASNSRIQTDE
(GmSTM Protein)
SEQ ID NO: 12
MEGSSCSNDTSYLLAFGENSGGLCPMTMMPLVTSHHATNPSNPSNNTNNN
ENTNCLFIPNCSNSSGTPSIMLHNNNNTDDDNNKTSTNTGLGYYFMESDH
HHRNNNNNGSSSSSSSSAVKAKIMAHPHYHRLLAAYVNCQKVGAPPEVVA
RLEEACASAATMAGDAAAAAGSSCIGEDPALDQFMEAYCEMLTKYEQELS
KPLKEAMLFLQRIECQFKNLTISSTDFACNEGAERNGSSEEDVDLHNMID
PQAEDRELKGQLLRKYSGYLGSLKQEFMKKRKKGKLPKEARQQLLEWWSR
HYKWPYPSESQKLALAESTGLDQKQINNWFINQRKRHWKPSEDMQFVVMD
PSHPHYYMDNVLGNPFPMDLSHPML
(GmE2FA Protein)
SEQ ID NO: 13
MSSAAGVPDRLASQPRGAAGAPALPPLKRHLAFVTKPPFAPPDEYHSFSS
ADSRRAADEAVVVRSPYMKRKSGMTDSEGESQAQKWSNSPGYTNVSNVTN
NSPFKTPVSAKGGRAQKAKASKEGRSCPPTPMSNAGSPSPLTPASSCRYD
SSLGLLTKKFINLVKHAEDGILDLNKAAETLEVQKRRIYDITNVLEGIGL
IEKKLKNRIHWKGIESSTSGEVDGDISVLKAEVEKLSLEEQGLDDQIREM
QERLRNLSENENNQKCLFVTEEDIKGLPCFQNETLIAIKAPHGTTLEVPD
PEEAVDYPQRRYRIILRSTMGPIDVYLISQFEEKFEEVNGAELPMIPLAS
SSGSNEQLMTEMVPAECSGKELEPQTQLSSHAFSDLNASQEFAGGMMKIV
PSDVDNDADYWLLSDADVSITDMWRTDSTVDWNGIDMLHPDFGIISRPQS
PSSGLAEVPSTGANSIQK
(GmAGL15 Protein)
SEQ ID NO: 14
MGRGKIEIKRIDNASSRQVTFSKRRTGLFKKAQELSILCDAEVAVIVFSN
TGKLFEFSSSGMKRTLSRYNKCLGSTDAAVAEIMTQKEDSKMVEILREEI
EKLETKQLQLVGKDLTGLGLKELQNLEQQLNEGLLSVKARKEELLMEQLE
QSRVQEQRVMLENETLRRQIEELRCLFPQSESMVPFQYQHTERKNTFVNT
GARCLNLANNCGNEKGSSDTAFHLGLPAGVQEEGPQERNLFK
(GmBBM1 Promoter)
SEQ ID NO: 15
AATATTATTAATATACTCTTAATATATTGGTTAATGAAATAAAATTAATT
ATTGATTTCTTAATTACTTATTCTTGAAGTATACAGATTCATAAAATCTC
TTCTTACAATGGACACAAAAACTAAGCATCTTTTCGTTTACAATGTGTCA
TTAGCATCTTCTTAATCTTCTTAATTAATGAATCTCTATTAGCGATTACA
ATGTGTCATTAACATCTTATTCGATAGTACTATTAATTGAGATTCCTCTC
ATTCAACCACTTTTATAAAAAAATAAAGTTTTAACAAAAAAGAAAATCAT
AGTTCATAATATCTAACTTTATACTTTATGAAAAAAAAGTAATGTATCAC
ATATCACATCAGAATTTATTTTCCATGAAACATGAAGGCAGTGATGCATC
AATCAGCACATTAGTGATTTTGTGTCACAAGTCACAACTGTTCAGAAAAA
GCTCTTAGAGTGAATCGTAACACCGTATCACAAGGGCGCATTATATTTTT
CAATACCGCGAGCAACTAGTAGTACTAGTGTGTTTGGACTACCACATTAA
TTACGAAATGGTCCCCGTGTGTGGATCTTTTCATTAGCCCTTGAAGTAAT
TTTTTTTTTCTGATTCAAAGATTTCAAGTGCCCTAGAATGTATAAGACGC
GTCCCATTTCTATTGTGTGCGCGTGTGTGGTGTGTACGTGCATATCAGCC
AGAAGAAAGAGAAAATAACTCAAAATATAGTAACTTAAAGTATACTATAA
ATGTTCTCTCATCTCTATGCTATAAATGTTTTTTTTTCAATTTTTTGAGC
TCTTCAAGAATTTGACCCTTCTCCTCCTCCTCCTTCTTCTTTTCTTTCAA
ACCTCCTCATATAAACTAGTACTATATGCTTCTTCTTCTTCTTCTCCTTC
ATGCACAAACTGCTATTTTCACCCTTTATATATCTATCTACTCCTGAAGA
TTAGATTACCTTGAGGGCTTTGTGCTCTCTGTGTAATATTCTTCAATATC
(GmWUS Promoter)
SEQ ID NO: 16
TGAAATGCCTATAGAATATGCGGACCAATGCACAACACAAAAAATAAATA
GCCCTGATGGAAAGGGAAATTCGATCTAAATCTACATCTCATCTTTTAAT
AAGTGTATGTACGGAAAGAGGAGAGATATAAAAAAAATAAAATAATAGAT
ATAATAAATTACTTATTTGATGAAAAATAAAAGTTAAAATATAAAAAGAG
AATTGAAGTAAAAGTGAGATGGAAAAAAAAAATGGATGTATCACCAATTG
ACCATAATAACTCTATATGCTTCATGCATTGGTTGGGACCCATGAAATGC
ACAATAAGTTCACAAATACATTTTTACCCTCCAATTCATCAGGTAAGTAC
AGAATATATATCTTGGTAGCTTGCTGATTCGACTTAATAATTATAGAGTA
AGAATTTAAAAAAAAAATGTATGTGTGTGTATAGGGGCCATGTCTGATAT
CTCCATCAAAAGAAGAACCTATTGAACTCCCAAATCACAACCCGCATCAT
TCCATTGCCATTCATTCATTCATTCAGAAAATCTACTCTTTTTTTTTTCT
TTCCTTCCATCCAATATATCATTTCATGCCTCATTTTTCTACCTTTTCCC
ACTGTCTCTGTGTGCAAATACTTTATTTCACACATACCTGGTCATGCCTT
TTCGTCCAAGTAATTCCTGATAGTACCCTCACTTTCTAAGCTCTCTTTTG
TCCCTTCCCTTTTTATGAACACCACTCTGTCACCCTCAGTCCTTCTCTCT
CAGATATTTATTTATGATTTTCTCTCTTTATCACTCCATGTACTATATGT
GCCTGTGCCTCATCTATCATCTATCATCTATCATCTATCATCACCTATTA
TAAGTTTATAACCCCCCTCACCCTTTCCTCCCCTTCATAATTCATGCAGT
AGTAATCTCTCTTCTCACCTATATACCCTCTAATATTCTAATTCTCTCTC
TTGATCCAACAAACAAACACTACCATTTTGTTTGTTCTGAGTAGTGATCC
(GmLEC2 Promoter)
SEQ ID NO: 17
ACACTTATTTTTTTCTTCAATCACATTCACGTATATTATTATATATTCTA
TAATATTTGTATTTATTCAATTCAATTATTTATTATTTTTTTATATTTAT
TAACATATATAAATGATAATTAAAAACATATTCAATTCAATAATAATATT
ATATATTATTATACACTAATTAATAAGTCACATTTATGTGTATATACCAA
TTGACTGTAATATTATCTTTTAGATTTTAATAAGTCACACACGCATGCAT
AAAGACGATTTTAATCAGACATATTCATGTATATTATCATATACTAATTA
ATAAATACCTATGTGATATTTTCATTGATTGCTTATGAAACTCTCAACCC
CACACATGAAGCCAAAACCATGGCCAAACCAAAACCCCAGCCATTTTCAC
ACCTCTATCTTCCCATAGTCACTTCCTATATTATTATCCTCTCTTCGTAA
CTGCAATTCATGTTCCTCTAGGCATCTTACAAACACATGGGGCACACACC
TTTCTTTGGCTTTATGCAACACATGAAGACAATGTCCATCTTGCATACCA
TTTATAAGTCAGCAAGTCTCAACTTTATGATACCATAACGCTCACTTTCA
CTGCAATGACATTTCATCTTCTCTTGTTTTTTCTGCTTCATCCATCTCAA
CACTCTCAATTTTTTTTTATATTTTGAACTTGCAATTTATGTGTTTTTGT
TCAGTGCATTTGATTACAACTCAGATGAGTATTCCAATGTCACAACGTTC
CCTCCACTTGTTACCCACTTCAACATCTTCCTTCCTCTCTCTTGTTTCCT
TTTCCTTCCTTTTCTTTATTCTCGTTCACAATCCTTGCATTTATTTTTGT
CATACTTTTTTTTTTATATTTTTGTTTGCTTAATTGGCACTACCACTGCA
CCTAAACAACTTCTTATAAGAGCCTCATACACACACACACTCTCTCAATT
CACTCAACACTCAAAAGAAAAACCTTGAAGCCTGTTAATTTCTCACCAAA
(GmGRF5 Promoter)
SEQ ID NO: 18
ATTATCATTGAGTTAAAACTCTAACTCAAGCATGAAAAAATACATTAAAG
TTTTGTGTTTTTCAATTACCATAAAGTTTGATGAATATTGGTTTTGACGT
TTTGTGGTTATGGAAATGATTAAGGAGAAAACATGTAAAGGGTTATGATG
GCCTATTGACAAGACGGTGGCCAATAGAGAGTTAAAGGCCAAATTGACTG
TAACCCAAATTCCACTGATGAAAGTGAGATGCTTGGGTTTGGGGGGTGAA
ATGAAAAAAGGAGAAAGGAGAAAGCATCAATCCGTGGCCAAAAAAAGCAG
GATTCAGCTCTAGCCTTGGCCTCCAAATCTATCAATGAGATAACGCCACG
CATGCTTCAAGCCAAAAAAGATTAAAAATGACACGTACGAGACTTTCTCT
TATTCAAAAAGTTACTACAATTGCAAAGAGAGATTGATAATTTGATATAC
TAATGGCCACTATTGCTCAGCAGCTTACACTTCACATAACCGGATGGCAT
GGCACTGTTTTCCATGAAGTGATGTGGAGACAGCAAAACCAAAGGTGCAT
GGACTAACATGCATTTGAATTTAATTTTTCTTCTTTTCCTTTGTACATTT
GTTTATGGATTTCTGTAAAGATGTTAGAGACAAGGGCAGCAACAAAGGCA
GCTGCAGAGAAAAAACAGAAGCAACAGAGGTGCAGTCATTATAAAGAGCA
GACTCACTCACTCACCCATCATCCAGCACATTAGAGAAATAGAGAGGAGG
TGGCAGCAAAGCCAGAAAGCATCATCAGACTCTCAGACCCATTAGTATTA
TCCGTGCACAGGAGAAGAATCTCTACCCTTGAAAAATATATATAAAAATA
AAATAATAATGACCCTCCAAAGTCCAAATTACTATCACCCCATCTAGAGA
ATTTATTTCACTCTTTCAAATCTTATATCTTCTTGTTCTTCACTTCCCCA
CTATTTTAGAGAGAGACACACACACTCTTCCTTCCTTTTGTTGTCTCAAA
(GmSTM Promoter)
SEQ ID NO: 19
TGCACATGCAATTTAATTGTGATATCATTATTATCACTCATATGAAGCTA
TTGCTAGCTCAAATAGTAGTATTAATTTATTATTAGAACTTTCAAGAACT
AAGCGTACGTTCAAGTATCAATCAATCAACACAATTTGCTCGATAATGAT
AACATACTCGTATACACCTAGCTCACATAAGTTACGGTATTAAACATTTA
TAATCTGACACAATTTAATATCATTATCGAGCTGTTATCATATTTAAGTT
AAGGATTTCTTTAATTAGTATTTTTAAGATATTAATTAAAAAAAATAAAA
AAATATTTATTGTGTAAATCAAGATAAAAAATTATATCTCTCAATAAAAA
TATTTTTACTTTAAATTTCTTAACTAATATTCTTAAAACACTTATTAATA
TTTATTTTTAGGTTAAAAGTAAAAGTATTTATAAGAAACAGTAATAGAAA
AATTAAATATATAATAGTTAATAATTAATAATTTGTTATTAAAATGACAT
CATACCTTACTGGCTCTTAGAAAATCAATTCTTATAGTTGTAGTACTTTT
TATAACAGAAAACATTATATTTCAAATTGAAGTGTACTCAAGAAAAAAAA
TGAAATGAAGAGTATAACCGGGAGAGGGGGACAATGGGAAGCGACAATGT
GTACGTAACCTGATGGAGGTGCTTTCACTACGGTATTTTACGGGAAGTGA
TGCTACGCTAGGCCTTTATTAATTATTATATTAGGGACGAGGGATATCAT
ATGGGATATAGAGATGAACTATGGTGCTGGAAATAGATCGAGAAAAAAGG
GGTTGCTGAGAGGAAGAGACATTCGGACTGTCCCACAAACTTTACCAGCT
TTATTTACTCACCTGCAGACGCGCTTTTTCCATGGTTAATTATACTGTAT
CGTATTAAATTAGATCATACTAGTATACTATATACTACCATAGGAAGAGA
GAGAAGTAAGCATCATCATATAGTAAATATTCATGTTTAGACTTTAGTAT
TAATAGTAACTAACGCTAATGTTAAAACACTAAATACATCTATTTTGGAG
CTAACAAGAAGAACAAATTAGGTTTGATAAATTAAATCCCTAATGTTCTG
TTAAATGTTGGTACTTGTTTGTGGGACTAGAGAATTTTTTAATCACTGTG
GTGAGAAGATCGAGGACAAATAGGGTGAGAATATTAAATGAGTGGAGGGA
TTGCCATCAAAGTGTAGAGAGAGAGAGAAGGAAGGGTTGATTTTGATTCC
GTGCCCCATAAACATAAACATAAACATAAACCATCTCATCTTTCTCCATT
GATGGCCAGTAGTGGGTAACTTGTTTTTCTTCCTCGATTTGATCGTTCCT
TCTCTCTCTCTATTGTGTTTTGTTTTATGCCAGGAATGGCAGCGTATCAG
TGGCAGTGCAGGAAAAGAGAGGGAGAGTTTTCATTGGGAAGGTAAAAGCT
TTTGTTTGTAGCAGTGAAACCTCGCCCCCTTCTCTTCATCGCTACTAGTA
GTAACTCATCGTTTTCTCGGTGTGCCCCGCGTGCGCTCTGCTGTGTCTTC
TCACTCACACCAGAGGTGTAACCGTGTAACCACTAGAATCATTTATTCAT
TAATGCTGGCAACAGTGGCATGGAAAGAAAGATTAATTTTTCCAAAGGAA
AGAAAAACCCTCTGCAGGCTTTGCCAGATAAGCCAAGTGGGAAAACCAAA
CCCTCTATTAGTACTTACTTCATGTAACTGACTATAGCCACCACTATCAC
TATTTAGGATTTTCTGTAAAAAGCCTGATACTCTTTTACCATAAAACCCG
GGAGAGCCCTGGAAGACAAACATCTTCATTCAGACTTCATAAAATAAAAT
AGAGAAGTGTTTTTTTGTTTTTTTGGTTTGTTGTAATTAAGGCTAGCTAG
TGAGTGTGTTCTACAACTGTAGTGAGCTACAGAAGGTGGTGGTAGTAGTA
GGCAAAAAGGATAAGACAGTGAGTGTGTATGTTGTTGACAAGCAAAAGCC
(GmE2FA Promoter)
SEQ ID NO: 20
AATTAGTCTTATTGAATACTTATAATTTAATAAGTTAACTTCCCAATTTT
AGATTATCAAATTTCTAGTTTCACGGAACATAACCTATTTTCAAAAATAA
TTTAACATAACACTTAATTTGGTATACTAACACACATGTACATTCATTAA
AAAATAGACTAAGTAATTGATAATATATTACAAAATTAAAACATATAAAC
TAATTATAAATTATTAAATATGATTTTATACCTGTGCTAGACATGTGGTA
TCACGCTAGTAATTAATAATATATTAAAAATTAAAATAATATAACAAGTT
ACTACTATAAATTATAAAATATAAATGTAATATCAATATAAGCCACAAGA
GTTAAACTTGTCCATATGTATAACTTTTAAGTAGTTAGAAAACTTGTTAA
AGATATAAAATTTATTGACGATATAAATTTTGTTTACACCAGTATCAATG
CATATCAATTAAATCCTTTTTCTATTAATTTTAACATATACATCACATTA
ATCACACTAATGAAGGTAAGCAAAGAATTTAACAAGTTTTTTTTTTTTTA
AAATCTAATATAAACTAAAAAGTAAGGCAGCGAAAAAGGAAATAAGATAA
TTTCATGATAATAATCTAAAAATACAATAACCCCGTACCAAAAAAACATG
TGTAATTACAGGAACACTTAAAATTTCTTCTTTTATTATTATTATTTTTT
TTTTCGCGCATGCAGTTCCCTCCACATCTATCCGAAACCAAATTCCCTCC
TTCCCTCGTTTTCTGCTCTCGCCTCCTCTACGTTCCATAACGCCCTCTCT
CTCTCTCTCTCTCTCTCTCTCTTTTTTTTTTTTTTTTCCAAACCCTTTTC
CCCTCCCTCTCACTTTCTCTCTCTAAACCCCACTCTTTCTCTCTCTAAAA
CCCTACACTGTACTCTCCTTCCTTCGGATCCTTCTCCCGTTTCCCTCCAA
TTTCCCCCCAATTCCGCTGGCCCCACCTCCGCCCCTTTTCCCGCTTCCTC
(GmAGL15 Promoter)
SEQ ID NO: 21
TCTAAATGCCCAGAGAACACAACACGGAGCCATGCAAAGTTGCCGTTTCC
AGCAAACCTCTCTGGTTATTTGAGGTAAAACGCTTTGCAGTCTCGCAAAT
CGCAACAACCCCTTCGTCTTCTCAGTAAAAGGGGTCTTACTTACTTAGTG
TCTTCGTTCGTATCTTCAACCCTGAATTCGCTTCTCCTCCCAAAGCACCA
CCACCACCTCTAATTAATTCCTCGTTCAGTTGGGCATGTTTGCGCATTTC
TGAGAGAGCGAGAAAATAAA
(VP128 CDS)
SEQ ID NO: 22
GGAGGGTCCGGAGGTGACGCTTTGGATGATTTCGATCTCGATATGCTCGG
CTCCGACGCCCTTGATGACTTCGACCTGGATATGCTTGGAAGCGACGCTC
TCGATGACTTCGATCTTGACATGCTTGGTAGTGATGCCCTGGACGACTTT
GACTTGGATATGCTCGCTCGGGGGTCCGACGCTTTGGATGACTTCGATCT
GGACATGCTGGGCTCAGACGCACTTGACGACTTCGACCTCGACATGCTGG
GATCAGACGCCCTCGATGATTTTGATCTTGACATGCTTGGAAGTGACGCG
TTGGACGATTTTGATCTCGATATGCTT
(6TAD CDS)
SEQ ID NO: 23
GGAGGGTCCGGAGGTCTGTTGGACCCTGGTACGCCTATGGATGCGGATTT
GGTCGCGTCTAGTACCGTTGTGTGGGAGCAAGACGCGGACCCGTTTGCAG
GAACAGCAGATGATTTTCCCGCGTTTAATGAAGAAGAGTTGGCCTGGTTG
ATGGAACTTCTTCCTCAGGGAGGTTCGGGAGGACTTCTTGACCCCGGCAC
TCCGATGGATGCCGACCTCGTCGCATCCTCTACTGTCGTTTGGGAACAGG
ATGCAGACCCGTTCGCAGGCACCGCAGATGATTTCCCTGCCTTTAACGAA
GAGGAACTCGCTTGGCTGATGGAATTGCTTCCGCAAGCGAGAGGGGGTTC
AGGCGGGTTGCTCGATCCGGGTACACCGATGGACGCCGACTTGGTTGCAT
CGTCAACAGTCGTCTGGGAACAGGACGCGGACCCCTTTGCGGGCACAGCG
GACGACTTCCCGGCTTTTAATGAGGAGGAACTCGCATGGCTTATGGAGCT
TTTGCCACAGGGTGGTTCAGGTGGTCTACTTGATCCTGGGACTCCTATGG
ACGCCGACTTGGTAGCTAGCTCAACAGTTGTTTGGGAGCAAGACGCTGAC
CCTTTCGCCGGCACTGCAGACGATTTTCCCGCTTTCAATGAAGAAGAGCT
CGCCTGGCTCATGGAGCTTCTGCCCCAGGCTAGAGGAGGCTCAGGTGGAT
TGCTGGATCCAGGCACCCCAATGGACGCAGATCTCGTCGCTAGTAGCACT
GTAGTGTGGGAACAGGATGCAGATCCCTTTGCTGGCACTGCCGACGACTT
CCCCGCATTCAACGAGGAGGAACTGGCTTGGCTTATGGAACTCCTCCCTC
AGGGGGGGTCCGGCGGCTTGCTGGATCCCGGCACTCCCATGGACGCAGAC
CTGGTTGCTTCTAGTACCGTCGTCTGGGAGCAAGACGCCGATCCATTCGC
AGGTACCGCCGATGATTTTCCTGCCTTTAATGAAGAAGAGTTGGCATGGT
TGATGGAGCTCCTTCCTCAA
(6TAD-VP128 CDS)
SEQ ID NO: 24
GGAGGGTCCGGAGGTCTGTTGGACCCTGGTACGCCTATGGATGCGGATTT
GGTCGCGTCTAGTACCGTTGTGTGGGAGCAAGACGCGGACCCGTTTGCAG
GAACAGCAGATGATTTTCCCGCGTTTAATGAAGAAGAGTTGGCCTGGTTG
ATGGAACTTCTTCCTCAGGGAGGTTCGGGAGGACTTCTTGACCCCGGCAC
TCCGATGGATGCCGACCTCGTCGCATCCTCTACTGTCGTTTGGGAACAGG
ATGCAGACCCGTTCGCAGGCACCGCAGATGATTTCCCTGCCTTTAACGAA
GAGGAACTCGCTTGGCTGATGGAATTGCTTCCGCAAGCGAGAGGGGGTTC
AGGCGGGTTGCTCGATCCGGGTACACCGATGGACGCCGACTTGGTTGCAT
CGTCAACAGTCGTCTGGGAACAGGACGCGGACCCCTTTGCGGGCACAGCG
GACGACTTCCCGGCTTTTAATGAGGAGGAACTCGCATGGCTTATGGAGCT
TTTGCCACAGGGTGGTTCAGGTGGTCTACTTGATCCTGGGACTCCTATGG
ACGCCGACTTGGTAGCTAGCTCAACAGTTGTTTGGGAGCAAGACGCTGAC
CCTTTCGCCGGCACTGCAGACGATTTTCCCGCTTTCAATGAAGAAGAGCT
CGCCTGGCTCATGGAGCTTCTGCCCCAGGCTAGAGGAGGCTCAGGTGGAT
TGCTGGATCCAGGCACCCCAATGGACGCAGATCTCGTCGCTAGTAGCACT
GTAGTGTGGGAACAGGATGCAGATCCCTTTGCTGGCACTGCCGACGACTT
CCCCGCATTCAACGAGGAGGAACTGGCTTGGCTTATGGAACTCCTCCCTC
AGGGGGGGTCCGGCGGCTTGCTGGATCCCGGCACTCCCATGGACGCAGAC
CTGGTTGCTTCTAGTACCGTCGTCTGGGAGCAAGACGCCGATCCATTCGC
AGGTACCGCCGATGATTTTCCTGCCTTTAATGAAGAAGAGTTGGCATGGT
TGATGGAGCTCCTTCCTCAAGCACGCGGGGGGTCTGGTGGTGGTGGATCT
GGCGGTGACGCTTTGGATGATTTCGATCTCGATATGCTCGGCTCCGACGC
CCTTGATGACTTCGACCTGGATATGCTTGGAAGCGACGCTCTCGATGACT
TCGATCTTGACATGCTTGGTAGTGATGCCCTGGACGACTTTGACTTGGAT
ATGCTCGCTCGGGGGTCCGACGCTTTGGATGACTTCGATCTGGACATGCT
GGGCTCAGACGCACTTGACGACTTCGACCTCGACATGCTGGGATCAGACG
CCCTCGATGATTTTGATCTTGACATGCTTGGAAGTGACGCGTTGGACGAT
TTTGATCTCGATATGCTT
(VP128 Protein)
SEQ ID NO: 25
GSDALDDFDLDMLGSDALDDFDLDMLGSDALDDFDLDMLGSDALDDFDLD
MLARGSDALDDFDLDMLGSDALDDFDLDMLGSDALDDFDLDMLGSDALDD
FDLDML
(6TAD Protein)
SEQ ID NO: 26
GGSGGLLDPGTPMDADLVASSTVVWEQDADPFAGTADDFPAFNEEELAWL
MELLPQGGSGGLLDPGTPMDADLVASSTVVWEQDADPFAGTADDFPAFNE
EELAWLMELLPQARGGSGGLLDPGTPMDADLVASSTVVWEQDADPFAGTA
DDFPAFNEEELAWLMELLPQGGSGGLLDPGTPMDADLVASSTVVWEQDAD
PFAGTADDFPAFNEEELAWLMELLPQARGGSGGLLDPGTPMDADLVASST
VVWEQDADPFAGTADDFPAFNEEELAWLMELLPQGGSGGLLDPGTPMDAD
LVASSTVVWEQDADPFAGTADDFPAFNEEELAWLMELLPQ
(6TAD-VP128 Protein)
SEQ ID NO: 27
GGSGGLLDPGTPMDADLVASSTVVWEQDADPFAGTADDFPAFNEEELAWL
MELLPQGGSGGLLDPGTPMDADLVASSTVVWEQDADPFAGTADDFPAFNE
EELAWLMELLPQARGGSGGLLDPGTPMDADLVASSTVVWEQDADPFAGTA
DDFPAFNEEELAWLMELLPQGGSGGLLDPGTPMDADLVASSTVVWEQDAD
PFAGTADDFPAFNEEELAWLMELLPQARGGSGGLLDPGTPMDADLVASST
VVWEQDADPFAGTADDFPAFNEEELAWLMELLPQGGSGGLLDPGTPMDAD
LVASSTVVWEQDADPFAGTADDFPAFNEEELAWLMELLPQARGGSGGGGS
GGDALDDFDLDMLGSDALDDFDLDMLGSDALDDFDLDMLGSDALDDFDLD
MLARGSDALDDFDLDMLGSDALDDFDLDMLGSDALDDFDLDMLGSDALDD
FDLDML
(full map of pCLS3)
SEQ ID NO: 28
CGATTCATTAATGCAGCTGGCACGACAGGTTTCCCGACTGGAAAGCGGGC
AGTGAGCGCAACGCAATTAATACGCGTACCGCTAGCCAGGAAGAGTTTGT
AGAAACGCAAAAAGGCCATCCGTCAGGATGGCCTTCTGCTTAGTTTGATG
CCTGGCAGTTTATGGCGGGCGTCCTGCCCGCCACCCTCCGGGCCGTTGCT
TCACAACGTTCAAATCCGCTCCCGGCGGATTTGTCCTACTCAGGAGAGCG
TTCACCGACAAACAACAGATAAAACGAAAGGCCCAGTCTTCCGACTGAGC
CTTTCGTTTTATTTGATGCCTGGCAGTTCCCTACTCTCGCGTTCGAATAC
ATCTAGATCCAAGTACATGGCAAATAATGATTTTATTTTGACTGATAGTG
ACCTGTTCGTTGCAACAAATTGATGAGCAATGCTTTTTTATAATGCCAAC
TTTGTACAAAAAAGCAGGCTTAGGTACCTCGCGAATGCATCTAGATCCAA
TGATCATGAGCGGAGAATTAAGGGAGTCACGTTATGACCCCCGCCGATGA
CGCGGGACAAGCCGTTTTACGTTTGGAACTGACAGAACCGCAACGTTGAA
GGAGCCACTCAGCCGCGGGTTTCTGGAGTTTAATGAGCTAAGCACATACG
TCAGAAACCATTATTGCGCGTTCAAAAGTCGCCTAAGGTCACTATCAGCT
AGCAAATATTTCTTGTCAAAAATGCTCCACTGACGTTCCATAAATTCCCC
TCGGTATCCAATTAGAGTCTCATATTCACTCTCAATCCAAATAATCTGCA
CCGGATCTCGCCCTTACCTGCTAGTCATGGGCGATCCTAAAAAGAAACGT
AAGGTCATCGATTACCCATACGATGTTCCAGATTACGCTATGGCTCCTAA
GAAGAAGAGAAAGGTTATAACAATGGTGAGCAAGGGCGAGGAGCTGTTCA
CCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCA
CCCTCGTGACCACCTTCGGCTACGGCCTGCAGTGCTTCGCCCGCTACCCC
GACCACATGAAGCAGCACGACTTCTTCAAGTCCGCCATGCCCGAAGGCTA
CGTCCAGGAGCGCACCATCTTCTTCAAGGACGACGGCAACTACAAGACCC
GCGCCGAGGTGAAGTTCGAGGGCGACACCCTGGTGAACCGCATCGAGCTG
AAGGGCATCGACTTCAAGGAGGACGGCAACATCCTGGGGCACAAGCTGGA
GTACAACTACAACAGCCACAACGTCTATATCATGGCCGACAAGCAGAAGA
ACGGCATCAAGGTGAACTTCAAGATCCGCCACAACATCGAGGACGGCAGC
GTGCAGCTCGCCGACCACTACCAGCAGAACACCCCCATCGGCGACGGCCC
CGTGCTGCTGCCCGACAACCACTACCTGAGCTACCAGTCCGCCCTGAGCA
AAGACCCCAACGAGAAGCGCGATCACATGGTCCTGCTGGAGTTCGTGACC
GCCGCCGGGATCACTCTCGGCATGGACGAGCTGTACAAGCCGCGGTTCCC
GGGAGATCTTGGAGGGGGCGGTAGCGGCGGTGGCGGGAGCATCGATATCG
CCGATCTACGCACGCTCGGCTACAGCCAGCAGCAACAGGAGAAGATCAAA
CCGAAGGTTCGTTCGACAGTGGCGCAGCACCACGAGGCACTGGTCGGCCA
CGGGTTTACACACGCGCACATCGTTGCGTTAAGCCAACACCCGGCAGCGT
TAGGGACCGTCGCTGTCAAGTATCAGGACATGATCGCAGCGTTGCCAGAG
GCGACACACGAAGCGATCGTTGGCGTCGGCAAACAGTGGTCCGGCGCACG
CGCTCTGGAGGCCTTGCTCACGGTGGCGGGAGAGTTGAGAGGTCCACCGT
TACAGTTGGACACAGGCCAACTTCTCAAGATTGCAAAACGTGGCGGCGTG
ACCGCAGTGGAGGCAGTGCATGCATGGCGCAATGCACTGACGGGTGCCCC
GCTCAACTTGACCCCCCAGCAGGTGGTGGCCATCGCCAGCAATAATGGTG
GCAAGCAGGCGCTGGAGACGGTCCAGCGGCTGTTGCCGGTGCTGTGCCAG
GCCCACGGCTTGACCCCGGAGCAGGTGGTGGCCATCGCCAGCCACGATGG
CGGCAAGCAGGCGCTGGAGACGGTCCAGCGGCTGTTGCCGGTGCTGTGCC
AGGCCCACGGCTTGACCCCCCAGCAGGTGGTGGCCATCGCCAGCAATAAT
GGTGGCAAGCAGGCGCTGGAGACGGTCCAGCGGCTGTTGCCGGTGCTGTG
CCAGGCCCACGGCTTGACCCCGGAGCAGGTGGTGGCCATCGCCAGCAATA
TTGGTGGCAAGCAGGCGCTGGAGACGGTGCAGGCGCTGTTGCCGGTGCTG
TGCCAGGCCCACGGCTTGACCCCCCAGCAGGTGGTGGCCATCGCCAGCAA
TAATGGTGGCAAGCAGGCGCTGGAGACGGTCCAGCGGCTGTTGCCGGTGC
TGTGCCAGGCCCACGGCTTGACCCCGGAGCAGGTGGTGGCCATCGCCAGC
AATATTGGTGGCAAGCAGGCGCTGGAGACGGTGCAGGCGCTGTTGCCGGT
GCTGTGCCAGGCCCACGGCTTGACCCCGGAGCAGGTGGTGGCCATCGCCA
GCCACGATGGCGGCAAGCAGGCGCTGGAGACGGTCCAGCGGCTGTTGCCG
GTGCTGTGCCAGGCCCACGGCTTGACCCCGGAGCAGGTGGTGGCCATCGC
CAGCAATATTGGTGGCAAGCAGGCGCTGGAGACGGTGCAGGCGCTGTTGC
CGGTGCTGTGCCAGGCCCACGGCTTGACCCCGGAGCAGGTGGTGGCCATC
GCCAGCCACGATGGCGGCAAGCAGGCGCTGGAGACGGTCCAGCGGCTGTT
GCCGGTGCTGTGCCAGGCCCACGGCTTGACCCCGGAGCAGGTGGTGGCCA
TCGCCAGCCACGATGGCGGCAAGCAGGCGCTGGAGACGGTCCAGCGGCTG
TTGCCGGTGCTGTGCCAGGCCCACGGCTTGACCCCCCAGCAGGTGGTGGC
CATCGCCAGCAATAATGGTGGCAAGCAGGCGCTGGAGACGGTCCAGCGGC
TGTTGCCGGTGCTGTGCCAGGCCCACGGCTTGACCCCGGAGCAGGTGGTG
GCCATCGCCAGCCACGATGGCGGCAAGCAGGCGCTGGAGACGGTCCAGCG
GCTGTTGCCGGTGCTGTGCCAGGCCCACGGCTTGACCCCGGAGCAGGTGG
TGGCCATCGCCAGCCACGATGGCGGCAAGCAGGCGCTGGAGACGGTCCAG
CGGCTGTTGCCGGTGCTGTGCCAGGCCCACGGCTTGACCCCGGAGCAGGT
GGTGGCCATCGCCAGCCACGATGGCGGCAAGCAGGCGCTGGAGACGGTCC
AGCGGCTGTTGCCGGTGCTGTGCCAGGCCCACGGCTTGACCCCCCAGCAG
GTGGTGGCCATCGCCAGCAATGGCGGTGGCAAGCAGGCGCTGGAGACGGT
CCAGCGGCTGTTGCCGGTGCTGTGCCAGGCCCACGGCTTGACCCCTCAGC
AGGTGGTGGCCATCGCCAGCAATGGCGGCGGCAGGCCGGCGCTGGAGAGC
ATTGTTGCCCAGTTATCTCGCCCTGATCCGGCGTTGGCCGCGTTGACCAA
CGACCACCTCGTCGCCTTGGCCTGCCTCGGCGGGCGTCCTGCGCTGGATG
CAGTGAAAAAGGGATTGGGGGATCCTATCAGCCGTTCCCAGCTGGTGAAG
TCCGAGCTGGAGGAGAAGAAATCCGAGTTGAGGCACAAGCTGAAGTACGT
GCCCCACGAGTACATCGAGCTGATCGAGATCGCCCGGAACAGCACCCAGG
ACCGTATCCTGGAGATGAAGGTGATGGAGTTCTTCATGAAGGTGTACGGC
TACAGGGGCAAGCACCTGGGCGGCTCCAGGAAGCCCGACGGCGCCATCTA
CACCGTGGGCTCCCCCATCGACTACGGCGTGATCGTGGACACCAAGGCCT
ACTCCGGCGGCTACAACCTGCCCATCGGCCAGGCCGACGAAATGCAGAGG
TACGTGGAGGAGAACCAGACCAGGAACAAGCACATCAACCCCAACGAGTG
GTGGAAGGTGTACCCCTCCAGCGTGACCGAGTTCAAGTTCCTGTTCGTGT
CCGGCCACTTCAAGGGCAACTACAAGGCCCAGCTGACCAGGCTGAACCAC
ATCACCAACTGCAACGGCGCCGTGCTGTCCGTGGAGGAGCTCCTGATCGG
CGGCGAGATGATCAAGGCCGGCACCCTGACCCTGGAGGAGGTGAGGAGGA
AGTTCAACAACGGCGAGATCAACTTCGCGGCCGACTGATAACTCGAGAAG
GGCGCGATCGTTCAAACATTTGGCAATAAAGTTTCTTAAGATTGAATCCT
GTTGCCGGTCTTGCGATGATTATCATATAATTTCTGTTGAATTACGTTAA
GCATGTAATAATTAACATGTAATGCATGACGTTATTTATGAGATGGGTTT
TTATGATTAGAGTCCCGCAATTATACATTTAATACGCGATAGAAAACAAA
ATATAGCGCGCAAACTAGGATAAATTATCGCGCGCGGTGTCATCTATGTT
ACTAGATCGGGAATTCGTAATCATGGTCATAGCATTGGATCGGATCCCGG
GCCCGTCGACTGCAGAGGCCTGCATGCAACAACTTTGTATACAAAAGTTG
AACGAGAAACGTAAAATGATATAAATATCAATATATTAAATTAGATTTTG
CATAAAAAACAGACTACATAATACTGTAAAACACAACATATCCAGTCACT
ATGTCGATTGTCTTCATCGGATCCCATCCCCTATAGTGAGTCGTATTACA
TGGTCATAGCTGTTTCCTGGCAGCTCTGGCCCGTGTCTCAAAATCTCTGA
TGTTACATTGCACAAGATAAAAATATATCATCATGCCTCCTCTAGACCAG
CCAGGACAGAAATGCCTCGACTTCGCTGCTGCCCAAGGTTGCCGGGTGAC
GCACACCGTGGAAACGGATGAAGGCACGAACCCAGTGGACATAAGCCTGT
TCGGTTCGTAAGCTGTAATGCAAGTAGCGTATGCGCTCACGCAACTGGTC
CAGAACCTTGACCGAACGCAGCGGTGGTAACGGCGCAGTGGCGGTTTTCA
TGGCTTGTTATGACTGTTTTTTTGGGGTACAGTCTATGCCTCGGGCATCC
AAGCAGCAAGCGCGTTACGCCGTGGGTCGATGTTTGATGTTATGGAGCAG
CAACGATGTTACGCAGCAGGGCAGTCGCCCTAAAACAAAGTTAAACATCA
TGAGGGAAGCGGTGATCGCCGAAGTATCGACTCAACTATCAGAGGTAGTT
GGCGTCATCGAGCGCCATCTCGAACCGACGTTGCTGGCCGTACATTTGTA
CGGCTCCGCAGTGGATGGCGGCCTGAAGCCACACAGTGATATTGATTTGC
TGGTTACGGTGACCGTAAGGCTTGATGAAACAACGCGGCGAGCTTTGATC
AACGACCTTTTGGAAACTTCGGCTTCCCCTGGAGAGAGCGAGATTCTCCG
CGCTGTAGAAGTCACCATTGTTGTGCACGACGACATCATTCCGTGGCGTT
ATCCAGCTAAGCGCGAACTGCAATTTGGAGAATGGCAGCGCAATGACATT
CTTGCAGGTATCTTCGAGCCAGCCACGATCGACATTGATCTGGCTATCTT
GCTGACAAAAGCAAGAGAACATAGCGTTGCCTTGGTAGGTCCAGCGGCGG
AGGAACTCTTTGATCCGGTTCCTGAACAGGATCTATTTGAGGCGCTAAAT
GAAACCTTAACGCTATGGAACTCGCCGCCCGACTGGGCTGGCGATGAGCG
AAATGTAGTGCTTACGTTGTCCCGCATTTGGTACAGCGCAGTAACCGGCA
AAATCGCGCCGAAGGATGTCGCTGCCGACTGGGCAATGGAGCGCCTGCCG
GCCCAGTATCAGCCCGTCATACTTGAAGCTAGACAGGCTTATCTTGGACA
AGAAGAAGATCGCTTGGCCTCGCGCGCAGATCAGTTGGAAGAATTTGTCC
ACTACGTGAAAGGCGAGATCACCAAGGTAGTCGGCAAATAACCCTCGAGC
CACCCATGACCAAAATCCCTTAACGTGAGTTACGCGTCGTTCCACTGAGC
GTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGG
CTTCAGCAGAGCGCAGATACCAAATACTGTCCTTCTAGTGTAGCCGTAGT
TAGGCCACCACTTCAAGAACTCTGTAGCACCGCCTACATACCTCGCTCTG
CTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCT
GAACGGGGGGTTCGTGCACACAGCCCAGCTTGGAGCGAACGACCTACACC
GAACTGAGATACCTACAGCGTGAGCATTGAGAAAGCGCCACGCTTCCCGA
AGGGAGAAAGGCGGACAGGTATCCGGTAAGCGGCAGGGTCGGAACAGGAG
AGCGCACGAGGGAGCTTCCAGGGGGAAACGCCTGGTATCTTTATAGTCCT
GTCGGGTTTCGCCACCTCTGACTTGAGCGTCGATTTTTGTGATGCTCGTC
AGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCC
CCTGATTCTGTGGATAACCGTATTACCGCCTTTGAGTGAGCTGATACCGC
TCGCCGCAGCCGAACGACCGAGCGCAGCGAGTCAGTGAGCGAGGAAGCGG
AAGAGCGCCCAATACGCAAACCGCCTCTCCCCGCGCGTTGGC
(expression cassette from pCLS3)
SEQ ID NO: 29
GATCATGAGCGGAGAATTAAGGGAGTCACGTTATGACCCCCGCCGATGAC
GCGGGACAAGCCGTTTTACGTTTGGAACTGACAGAACCGCAACGTTGAAG
GAGCCACTCAGCCGCGGGTTTCTGGAGTTTAATGAGCTAAGCACATACGT
CAGAAACCATTATTGCGCGTTCAAAAGTCGCCTAAGGTCACTATCAGCTA
GCAAATATTTCTTGTCAAAAATGCTCCACTGACGTTCCATAAATTCCCCT
CGGTATCCAATTAGAGTCTCATATTCACTCTCAATCCAAATAATCTGCAC
CGGATCTCGCCCTTACCTGCTAGTCATGGGCGATCCTAAAAAGAAACGTA
AGGTCATCGATTACCCATACGATGTTCCAGATTACGCTATGGCTCCTAAG
AAGAAGAGAAAGGTTATAACAATGGTGAGCAAGGGCGAGGAGCTGTTCAC
CGGGGTGGTGCCCATCCTGGTCGAGCTGGACGGCGACGTAAACGGCCACA
AGTTCAGCGTGTCCGGCGAGGGCGAGGGCGATGCCACCTACGGCAAGCTG
ACCCTGAAGTTCATCTGCACCACCGGCAAGCTGCCCGTGCCCTGGCCCAC
CCTCGTGACCACCTTCGGCTACGGCCTGCAGTGCTTCGCCCGCTACCCCG
ACCACATGAAGCAGCACGACTTCTTCAAGTCCGCCATGCCCGAAGGCTAC
GTCCAGGAGCGCACCATCTTCTTCAAGGACGACGGCAACTACAAGACCCG
CGCCGAGGTGAAGTTCGAGGGCGACACCCTGGTGAACCGCATCGAGCTGA
AGGGCATCGACTTCAAGGAGGACGGCAACATCCTGGGGCACAAGCTGGAG
TACAACTACAACAGCCACAACGTCTATATCATGGCCGACAAGCAGAAGAA
CGGCATCAAGGTGAACTTCAAGATCCGCCACAACATCGAGGACGGCAGCG
TGCAGCTCGCCGACCACTACCAGCAGAACACCCCCATCGGCGACGGCCCC
GTGCTGCTGCCCGACAACCACTACCTGAGCTACCAGTCCGCCCTGAGCAA
AGACCCCAACGAGAAGCGCGATCACATGGTCCTGCTGGAGTTCGTGACCG
CCGCCGGGATCACTCTCGGCATGGACGAGCTGTACAAGCCGCGGTTCCCG
GGAGATCTTGGAGGGGGCGGTAGCGGCGGTGGCGGGAGCATCGATATCGC
CGATCTACGCACGCTCGGCTACAGCCAGCAGCAACAGGAGAAGATCAAAC
CGAAGGTTCGTTCGACAGTGGCGCAGCACCACGAGGCACTGGTCGGCCAC
GGGTTTACACACGCGCACATCGTTGCGTTAAGCCAACACCCGGCAGCGTT
AGGGACCGTCGCTGTCAAGTATCAGGACATGATCGCAGCGTTGCCAGAGG
CGACACACGAAGCGATCGTTGGCGTCGGCAAACAGTGGTCCGGCGCACGC
GCTCTGGAGGCCTTGCTCACGGTGGCGGGAGAGTTGAGAGGTCCACCGTT
ACAGTTGGACACAGGCCAACTTCTCAAGATTGCAAAACGTGGCGGCGTGA
CCGCAGTGGAGGCAGTGCATGCATGGCGCAATGCACTGACGGGTGCCCCG
CTCAACTTGACCCCCCAGCAGGTGGTGGCCATCGCCAGCAATAATGGTGG
CAAGCAGGCGCTGGAGACGGTCCAGCGGCTGTTGCCGGTGCTGTGCCAGG
CCCACGGCTTGACCCCGGAGCAGGTGGTGGCCATCGCCAGCCACGATGGC
GGCAAGCAGGCGCTGGAGACGGTCCAGCGGCTGTTGCCGGTGCTGTGCCA
GGCCCACGGCTTGACCCCCCAGCAGGTGGTGGCCATCGCCAGCAATAATG
GTGGCAAGCAGGCGCTGGAGACGGTCCAGCGGCTGTTGCCGGTGCTGTGC
CAGGCCCACGGCTTGACCCCGGAGCAGGTGGTGGCCATCGCCAGCAATAT
TGGTGGCAAGCAGGCGCTGGAGACGGTGCAGGCGCTGTTGCCGGTGCTGT
GCCAGGCCCACGGCTTGACCCCCCAGCAGGTGGTGGCCATCGCCAGCAAT
AATGGTGGCAAGCAGGCGCTGGAGACGGTCCAGCGGCTGTTGCCGGTGCT
GTGCCAGGCCCACGGCTTGACCCCGGAGCAGGTGGTGGCCATCGCCAGCA
ATATTGGTGGCAAGCAGGCGCTGGAGACGGTGCAGGCGCTGTTGCCGGTG
CTGTGCCAGGCCCACGGCTTGACCCCGGAGCAGGTGGTGGCCATCGCCAG
CCACGATGGCGGCAAGCAGGCGCTGGAGACGGTCCAGCGGCTGTTGCCGG
TGCTGTGCCAGGCCCACGGCTTGACCCCGGAGCAGGTGGTGGCCATCGCC
AGCAATATTGGTGGCAAGCAGGCGCTGGAGACGGTGCAGGCGCTGTTGCC
GGTGCTGTGCCAGGCCCACGGCTTGACCCCGGAGCAGGTGGTGGCCATCG
CCAGCCACGATGGCGGCAAGCAGGCGCTGGAGACGGTCCAGCGGCTGTTG
CCGGTGCTGTGCCAGGCCCACGGCTTGACCCCGGAGCAGGTGGTGGCCAT
CGCCAGCCACGATGGCGGCAAGCAGGCGCTGGAGACGGTCCAGCGGCTGT
TGCCGGTGCTGTGCCAGGCCCACGGCTTGACCCCCCAGCAGGTGGTGGCC
ATCGCCAGCAATAATGGTGGCAAGCAGGCGCTGGAGACGGTCCAGCGGCT
GTTGCCGGTGCTGTGCCAGGCCCACGGCTTGACCCCGGAGCAGGTGGTGG
CCATCGCCAGCCACGATGGCGGCAAGCAGGCGCTGGAGACGGTCCAGCGG
CTGTTGCCGGTGCTGTGCCAGGCCCACGGCTTGACCCCGGAGCAGGTGGT
GGCCATCGCCAGCCACGATGGCGGCAAGCAGGCGCTGGAGACGGTCCAGC
GGCTGTTGCCGGTGCTGTGCCAGGCCCACGGCTTGACCCCGGAGCAGGTG
GTGGCCATCGCCAGCCACGATGGCGGCAAGCAGGCGCTGGAGACGGTCCA
GCGGCTGTTGCCGGTGCTGTGCCAGGCCCACGGCTTGACCCCCCAGCAGG
TGGTGGCCATCGCCAGCAATGGCGGTGGCAAGCAGGCGCTGGAGACGGTC
CAGCGGCTGTTGCCGGTGCTGTGCCAGGCCCACGGCTTGACCCCTCAGCA
GGTGGTGGCCATCGCCAGCAATGGCGGCGGCAGGCCGGCGCTGGAGAGCA
TTGTTGCCCAGTTATCTCGCCCTGATCCGGCGTTGGCCGCGTTGACCAAC
GACCACCTCGTCGCCTTGGCCTGCCTCGGCGGGCGTCCTGCGCTGGATGC
AGTGAAAAAGGGATTGGGGGATCCTATCAGCCGTTCCCAGCTGGTGAAGT
CCGAGCTGGAGGAGAAGAAATCCGAGTTGAGGCACAAGCTGAAGTACGTG
CCCCACGAGTACATCGAGCTGATCGAGATCGCCCGGAACAGCACCCAGGA
CCGTATCCTGGAGATGAAGGTGATGGAGTTCTTCATGAAGGTGTACGGCT
ACAGGGGCAAGCACCTGGGCGGCTCCAGGAAGCCCGACGGCGCCATCTAC
ACCGTGGGCTCCCCCATCGACTACGGCGTGATCGTGGACACCAAGGCCTA
CTCCGGCGGCTACAACCTGCCCATCGGCCAGGCCGACGAAATGCAGAGGT
ACGTGGAGGAGAACCAGACCAGGAACAAGCACATCAACCCCAACGAGTGG
TGGAAGGTGTACCCCTCCAGCGTGACCGAGTTCAAGTTCCTGTTCGTGTC
CGGCCACTTCAAGGGCAACTACAAGGCCCAGCTGACCAGGCTGAACCACA
TCACCAACTGCAACGGCGCCGTGCTGTCCGTGGAGGAGCTCCTGATCGGC
GGCGAGATGATCAAGGCCGGCACCCTGACCCTGGAGGAGGTGAGGAGGAA
GTTCAACAACGGCGAGATCAACTTCGCGGCCGACTGATAACTCGAGAAGG
GCGCGATCGTTCAAACATTTGGCAATAAAGTTTCTTAAGATTGAATCCTG
TTGCCGGTCTTGCGATGATTATCATATAATTTCTGTTGAATTACGTTAAG
CATGTAATAATTAACATGTAATGCATGACGTTATTTATGAGATGGGTTTT
TATGATTAGAGTCCCGCAATTATACATTTAATACGCGATAGAAAACAAAA
TATAGCGCGCAAACTAGGATAAATTATCGCGCGCGGTGTCATCTATGTTA
CTAGATCGGGAATTCGTAATCATGGTCATAGC
(full map of pCLS4)
SEQ ID NO: 30
CGATTCATTAATGCAGCTGGCACGACAGGTTTCCCGACTGGAAAGCGGGC
AGTGAGCGCAACGCAATTAATACGCGTACCGCTAGCCAGGAAGAGTTTGT
AGAAACGCAAAAAGGCCATCCGTCAGGATGGCCTTCTGCTTAGTTTGATG
CCTGGCAGTTTATGGCGGGCGTCCTGCCCGCCACCCTCCGGGCCGTTGCT
TCACAACGTTCAAATCCGCTCCCGGCGGATTTGTCCTACTCAGGAGAGCG
TTCACCGACAAACAACAGATAAAACGAAAGGCCCAGTCTTCCGACTGAGC
CTTTCGTTTTATTTGATGCCTGGCAGTTCCCTACTCTCGCGTTCGAATAC
ATCTAGATCCAAGTACATGGCAAATAATGATTTTATTTTGACTGATAGTG
ACCTGTTCGTTGCAACAAATTGATGAGCAATGCTTTTTTATAATGCCAAC
TTTGTATACAAAAGTTGTAGGTACCTCGCGAATGCATCTAGATCCAATGA
TCATGAGCGGAGAATTAAGGGAGTCACGTTATGACCCCCGCCGATGACGC
GGGACAAGCCGTTTTACGTTTGGAACTGACAGAACCGCAACGTTGAAGGA
GCCACTCAGCCGCGGGTTTCTGGAGTTTAATGAGCTAAGCACATACGTCA
GAAACCATTATTGCGCGTTCAAAAGTCGCCTAAGGTCACTATCAGCTAGC
AAATATTTCTTGTCAAAAATGCTCCACTGACGTTCCATAAATTCCCCTCG
GTATCCAATTAGAGTCTCATATTCACTCTCAATCCAAATAATCTGCACCG
GATCTCGCCCTTACCTGCTAGTCATGGGCGATCCTAAAAAGAAACGTAAG
GTCATCGATAAGGAGACTGCCGCTGCCAAGTTCGAGAGACAGCACATGGA
CAGCATGGTGTCTAAGGGCGAAGAGCTGATTAAGGAGAACATGCACATGA
AGCTGTACATGGAGGGCACCGTGAACAACCACCACTTCAAGTGCACATCC
GAGGGCGAAGGCAAGCCCTACGAGGGCACCCAGACCATGAGAATCAAGGT
GGTCGAGGGCGGCCCTCTCCCCTTCGCCTTCGACATCCTGGCTACCAGCT
TCATGTACGGCAGCAGAACCTTCATCAACCACACCCAGGGCATCCCCGAC
TTCTTTAAGCAGTCCTTCCCTGAGGGCTTCACATGGGAGAGAGTCACCAC
ATACGAAGACGGGGGCGTGCTGACCGCTACCCAGGACACCAGCCTCCAGG
ACGGCTGCCTCATCTACAACGTCAAGATCAGAGGGGTGAACTTCCCATCC
AACGGCCCTGTGATGCAGAAGAAAACACTCGGCTGGGAGGCCAACACCGA
GATGCTGTACCCCGCTGACGGCGGCCTGGAAGGCAGAAGCGACATGGCCC
TGAAGCTCGTGGGCGGGGGCCACCTGATCTGCAACTTCAAGACCACATAC
AGATCCAAGAAACCCGCTAAGAACCTCAAGATGCCCGGCGTCTACTATGT
GGACCACAGACTGGAAAGAATCAAGGAGGCCGACAAAGAGACGTACGTCG
AGCAGCACGAGGTGGCTGTGGCCAGATACTGCGACCTCCCTAGCAAACTG
GGGCACAAACTTAATGGAGGGGGCGGTAGCGGCGGTGGCGGGAGCATCGA
TATCGCCGATCTACGCACGCTCGGCTACAGCCAGCAGCAACAGGAGAAGA
TCAAACCGAAGGTTCGTTCGACAGTGGCGCAGCACCACGAGGCACTGGTC
GGCCACGGGTTTACACACGCGCACATCGTTGCGTTAAGCCAACACCCGGC
AGCGTTAGGGACCGTCGCTGTCAAGTATCAGGACATGATCGCAGCGTTGC
CAGAGGCGACACACGAAGCGATCGTTGGCGTCGGCAAACAGTGGTCCGGC
GCACGCGCTCTGGAGGCCTTGCTCACGGTGGCGGGAGAGTTGAGAGGTCC
ACCGTTACAGTTGGACACAGGCCAACTTCTCAAGATTGCAAAACGTGGCG
GCGTGACCGCAGTGGAGGCAGTGCATGCATGGCGCAATGCACTGACGGGT
GCCCCGCTCAACTTGACCCCCCAGCAGGTGGTGGCCATCGCCAGCAATAA
TGGTGGCAAGCAGGCGCTGGAGACGGTCCAGCGGCTGTTGCCGGTGCTGT
GCCAGGCCCACGGCTTGACCCCCCAGCAGGTGGTGGCCATCGCCAGCAAT
AATGGTGGCAAGCAGGCGCTGGAGACGGTCCAGCGGCTGTTGCCGGTGCT
GTGCCAGGCCCACGGCTTGACCCCCCAGCAGGTGGTGGCCATCGCCAGCA
ATAATGGTGGCAAGCAGGCGCTGGAGACGGTCCAGCGGCTGTTGCCGGTG
CTGTGCCAGGCCCACGGCTTGACCCCGGAGCAGGTGGTGGCCATCGCCAG
CAATATTGGTGGCAAGCAGGCGCTGGAGACGGTGCAGGCGCTGTTGCCGG
TGCTGTGCCAGGCCCACGGCTTGACCCCCCAGCAGGTGGTGGCCATCGCC
AGCAATGGCGGTGGCAAGCAGGCGCTGGAGACGGTCCAGCGGCTGTTGCC
GGTGCTGTGCCAGGCCCACGGCTTGACCCCCCAGCAGGTGGTGGCCATCG
CCAGCAATGGCGGTGGCAAGCAGGCGCTGGAGACGGTCCAGCGGCTGTTG
CCGGTGCTGTGCCAGGCCCACGGCTTGACCCCCCAGCAGGTGGTGGCCAT
CGCCAGCAATAATGGTGGCAAGCAGGCGCTGGAGACGGTCCAGCGGCTGT
TGCCGGTGCTGTGCCAGGCCCACGGCTTGACCCCGGAGCAGGTGGTGGCC
ATCGCCAGCCACGATGGCGGCAAGCAGGCGCTGGAGACGGTCCAGCGGCT
GTTGCCGGTGCTGTGCCAGGCCCACGGCTTGACCCCCCAGCAGGTGGTGG
CCATCGCCAGCAATGGCGGTGGCAAGCAGGCGCTGGAGACGGTCCAGCGG
CTGTTGCCGGTGCTGTGCCAGGCCCACGGCTTGACCCCCCAGCAGGTGGT
GGCCATCGCCAGCAATGGCGGTGGCAAGCAGGCGCTGGAGACGGTCCAGC
GGCTGTTGCCGGTGCTGTGCCAGGCCCACGGCTTGACCCCCCAGCAGGTG
GTGGCCATCGCCAGCAATGGCGGTGGCAAGCAGGCGCTGGAGACGGTCCA
GCGGCTGTTGCCGGTGCTGTGCCAGGCCCACGGCTTGACCCCGGAGCAGG
TGGTGGCCATCGCCAGCCACGATGGCGGCAAGCAGGCGCTGGAGACGGTC
CAGCGGCTGTTGCCGGTGCTGTGCCAGGCCCACGGCTTGACCCCCCAGCA
GGTGGTGGCCATCGCCAGCAATGGCGGTGGCAAGCAGGCGCTGGAGACGG
TCCAGCGGCTGTTGCCGGTGCTGTGCCAGGCCCACGGCTTGACCCCCCAG
CAGGTGGTGGCCATCGCCAGCAATGGCGGTGGCAAGCAGGCGCTGGAGAC
GGTCCAGCGGCTGTTGCCGGTGCTGTGCCAGGCCCACGGCTTGACCCCCC
AGCAGGTGGTGGCCATCGCCAGCAATAATGGTGGCAAGCAGGCGCTGGAG
ACGGTCCAGCGGCTGTTGCCGGTGCTGTGCCAGGCCCACGGCTTGACCCC
TCAGCAGGTGGTGGCCATCGCCAGCAATGGCGGCGGCAGGCCGGCGCTGG
AGAGCATTGTTGCCCAGTTATCTCGCCCTGATCCGGCGTTGGCCGCGTTG
ACCAACGACCACCTCGTCGCCTTGGCCTGCCTCGGCGGGCGTCCTGCGCT
GGATGCAGTGAAAAAGGGATTGGGGGATCCTATCAGCCGTTCCCAGCTGG
TGAAGTCCGAGCTGGAGGAGAAGAAATCCGAGTTGAGGCACAAGCTGAAG
TACGTGCCCCACGAGTACATCGAGCTGATCGAGATCGCCCGGAACAGCAC
CCAGGACCGTATCCTGGAGATGAAGGTGATGGAGTTCTTCATGAAGGTGT
ACGGCTACAGGGGCAAGCACCTGGGCGGCTCCAGGAAGCCCGACGGCGCC
ATCTACACCGTGGGCTCCCCCATCGACTACGGCGTGATCGTGGACACCAA
GGCCTACTCCGGCGGCTACAACCTGCCCATCGGCCAGGCCGACGAAATGC
AGAGGTACGTGGAGGAGAACCAGACCAGGAACAAGCACATCAACCCCAAC
GAGTGGTGGAAGGTGTACCCCTCCAGCGTGACCGAGTTCAAGTTCCTGTT
CGTGTCCGGCCACTTCAAGGGCAACTACAAGGCCCAGCTGACCAGGCTGA
ACCACATCACCAACTGCAACGGCGCCGTGCTGTCCGTGGAGGAGCTCCTG
ATCGGCGGCGAGATGATCAAGGCCGGCACCCTGACCCTGGAGGAGGTGAG
GAGGAAGTTCAACAACGGCGAGATCAACTTCGCGGCCGACTGATAACTCG
AGAAGGGCGCGATCGTTCAAACATTTGGCAATAAAGTTTCTTAAGATTGA
ATCCTGTTGCCGGTCTTGCGATGATTATCATATAATTTCTGTTGAATTAC
GTTAAGCATGTAATAATTAACATGTAATGCATGACGTTATTTATGAGATG
GGTTTTTATGATTAGAGTCCCGCAATTATACATTTAATACGCGATAGAAA
ACAAAATATAGCGCGCAAACTAGGATAAATTATCGCGCGCGGTGTCATCT
ATGTTACTAGATCGGGAATTCGTAATCATGGTCATAGCATTGGATCGGAT
CCCGGGCCCGTCGACTGCAGAGGCCTGCATGCAAACCAGCTTTCTTGTAC
AAAGTTGGCATTATAAGAAAGCATTGCTTATCAATTTGTTGCAACGAACA
GGTCACTATCAGTCAAAATAAAATCATTATTTGTCGATTGTCTTCATCGG
ATCCCATCCCCTATAGTGAGTCGTATTACATGGTCATAGCTGTTTCCTGG
CAGCTCTGGCCCGTGTCTCAAAATCTCTGATGTTACATTGCACAAGATAA
AAATATATCATCATGCCTCCTCTAGACCAGCCAGGACAGAAATGCCTCGA
CTTCGCTGCTGCCCAAGGTTGCCGGGTGACGCACACCGTGGAAACGGATG
AAGGCACGAACCCAGTGGACATAAGCCTGTTCGGTTCGTAAGCTGTAATG
CAAGTAGCGTATGCGCTCACGCAACTGGTCCAGAACCTTGACCGAACGCA
GCGGTGGTAACGGCGCAGTGGCGGTTTTCATGGCTTGTTATGACTGTTTT
TTTGGGGTACAGTCTATGCCTCGGGCATCCAAGCAGCAAGCGCGTTACGC
CGTGGGTCGATGTTTGATGTTATGGAGCAGCAACGATGTTACGCAGCAGG
GCAGTCGCCCTAAAACAAAGTTAAACATCATGAGGGAAGCGGTGATCGCC
GAAGTATCGACTCAACTATCAGAGGTAGTTGGCGTCATCGAGCGCCATCT
CGAACCGACGTTGCTGGCCGTACATTTGTACGGCTCCGCAGTGGATGGCG
GCCTGAAGCCACACAGTGATATTGATTTGCTGGTTACGGTGACCGTAAGG
CTTGATGAAACAACGCGGCGAGCTTTGATCAACGACCTTTTGGAAACTTC
GGCTTCCCCTGGAGAGAGCGAGATTCTCCGCGCTGTAGAAGTCACCATTG
TTGTGCACGACGACATCATTCCGTGGCGTTATCCAGCTAAGCGCGAACTG
CAATTTGGAGAATGGCAGCGCAATGACATTCTTGCAGGTATCTTCGAGCC
AGCCACGATCGACATTGATCTGGCTATCTTGCTGACAAAAGCAAGAGAAC
ATAGCGTTGCCTTGGTAGGTCCAGCGGCGGAGGAACTCTTTGATCCGGTT
CCTGAACAGGATCTATTTGAGGCGCTAAATGAAACCTTAACGCTATGGAA
CTCGCCGCCCGACTGGGCTGGCGATGAGCGAAATGTAGTGCTTACGTTGT
CCCGCATTTGGTACAGCGCAGTAACCGGCAAAATCGCGCCGAAGGATGTC
GCTGCCGACTGGGCAATGGAGCGCCTGCCGGCCCAGTATCAGCCCGTCAT
ACTTGAAGCTAGACAGGCTTATCTTGGACAAGAAGAAGATCGCTTGGCCT
CGCGCGCAGATCAGTTGGAAGAATTTGTCCACTACGTGAAAGGCGAGATC
ACCAAGGTAGTCGGCAAATAACCCTCGAGCCACCCATGACCAAAATCCCT
TAACGTGAGTTACGCGTCGTTCCACTGAGCGTCAGACCCCGTAGAAAAGA
TCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGA
GCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAAC
TCTGTAGCACCGCCTACATACCTCGCTCTGCTAATCCTGTTACCAGTGGC
TGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACA
CAGCCCAGCTTGGAGCGAACGACCTACACCGAACTGAGATACCTACAGCG
TGAGCATTGAGAAAGCGCCACGCTTCCCGAAGGGAGAAAGGCGGACAGGT
ATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCT
TTTGCTCACATGTTCTTTCCTGCGTTATCCCCTGATTCTGTGGATAACCG
TATTACCGCCTTTGAGTGAGCTGATACCGCTCGCCGCAGCCGAACGACCG
AGCGCAGCGAGTCAGTGAGCGAGGAAGCGGAAGAGCGCCCAATACGCAAA
CCGCCTCTCCCCGCGCGTTGGC
(expression cassette from pCLS4)
SEQ ID NO: 31
GATCATGAGCGGAGAATTAAGGGAGTCACGTTATGACCCCCGCCGATGAC
GCGGGACAAGCCGTTTTACGTTTGGAACTGACAGAACCGCAACGTTGAAG
GAGCCACTCAGCCGCGGGTTTCTGGAGTTTAATGAGCTAAGCACATACGT
CAGAAACCATTATTGCGCGTTCAAAAGTCGCCTAAGGTCACTATCAGCTA
GCAAATATTTCTTGTCAAAAATGCTCCACTGACGTTCCATAAATTCCCCT
CGGTATCCAATTAGAGTCTCATATTCACTCTCAATCCAAATAATCTGCAC
CGGATCTCGCCCTTACCTGCTAGTCATGGGCGATCCTAAAAAGAAACGTA
AGGTCATCGATAAGGAGACTGCCGCTGCCAAGTTCGAGAGACAGCACATG
GACAGCATGGTGTCTAAGGGCGAAGAGCTGATTAAGGAGAACATGCACAT
GAAGCTGTACATGGAGGGCACCGTGAACAACCACCACTTCAAGTGCACAT
CCGAGGGCGAAGGCAAGCCCTACGAGGGCACCCAGACCATGAGAATCAAG
GTGGTCGAGGGCGGCCCTCTCCCCTTCGCCTTCGACATCCTGGCTACCAG
CTTCATGTACGGCAGCAGAACCTTCATCAACCACACCCAGGGCATCCCCG
ACTTCTTTAAGCAGTCCTTCCCTGAGGGCTTCACATGGGAGAGAGTCACC
ACATACGAAGACGGGGGCGTGCTGACCGCTACCCAGGACACCAGCCTCCA
GGACGGCTGCCTCATCTACAACGTCAAGATCAGAGGGGTGAACTTCCCAT
CCAACGGCCCTGTGATGCAGAAGAAAACACTCGGCTGGGAGGCCAACACC
GAGATGCTGTACCCCGCTGACGGCGGCCTGGAAGGCAGAAGCGACATGGC
CCTGAAGCTCGTGGGCGGGGGCCACCTGATCTGCAACTTCAAGACCACAT
ACAGATCCAAGAAACCCGCTAAGAACCTCAAGATGCCCGGCGTCTACTAT
GTGGACCACAGACTGGAAAGAATCAAGGAGGCCGACAAAGAGACGTACGT
CGAGCAGCACGAGGTGGCTGTGGCCAGATACTGCGACCTCCCTAGCAAAC
TGGGGCACAAACTTAATGGAGGGGGCGGTAGCGGCGGTGGCGGGAGCATC
GATATCGCCGATCTACGCACGCTCGGCTACAGCCAGCAGCAACAGGAGAA
GATCAAACCGAAGGTTCGTTCGACAGTGGCGCAGCACCACGAGGCACTGG
TCGGCCACGGGTTTACACACGCGCACATCGTTGCGTTAAGCCAACACCCG
GCAGCGTTAGGGACCGTCGCTGTCAAGTATCAGGACATGATCGCAGCGTT
GCCAGAGGCGACACACGAAGCGATCGTTGGCGTCGGCAAACAGTGGTCCG
GCGCACGCGCTCTGGAGGCCTTGCTCACGGTGGCGGGAGAGTTGAGAGGT
CCACCGTTACAGTTGGACACAGGCCAACTTCTCAAGATTGCAAAACGTGG
CGGCGTGACCGCAGTGGAGGCAGTGCATGCATGGCGCAATGCACTGACGG
GTGCCCCGCTCAACTTGACCCCCCAGCAGGTGGTGGCCATCGCCAGCAAT
AATGGTGGCAAGCAGGCGCTGGAGACGGTCCAGCGGCTGTTGCCGGTGCT
GTGCCAGGCCCACGGCTTGACCCCCCAGCAGGTGGTGGCCATCGCCAGCA
ATAATGGTGGCAAGCAGGCGCTGGAGACGGTCCAGCGGCTGTTGCCGGTG
CTGTGCCAGGCCCACGGCTTGACCCCCCAGCAGGTGGTGGCCATCGCCAG
CAATAATGGTGGCAAGCAGGCGCTGGAGACGGTCCAGCGGCTGTTGCCGG
TGCTGTGCCAGGCCCACGGCTTGACCCCGGAGCAGGTGGTGGCCATCGCC
AGCAATATTGGTGGCAAGCAGGCGCTGGAGACGGTGCAGGCGCTGTTGCC
GGTGCTGTGCCAGGCCCACGGCTTGACCCCCCAGCAGGTGGTGGCCATCG
CCAGCAATGGCGGTGGCAAGCAGGCGCTGGAGACGGTCCAGCGGCTGTTG
CCGGTGCTGTGCCAGGCCCACGGCTTGACCCCCCAGCAGGTGGTGGCCAT
CGCCAGCAATGGCGGTGGCAAGCAGGCGCTGGAGACGGTCCAGCGGCTGT
TGCCGGTGCTGTGCCAGGCCCACGGCTTGACCCCCCAGCAGGTGGTGGCC
ATCGCCAGCAATAATGGTGGCAAGCAGGCGCTGGAGACGGTCCAGCGGCT
GTTGCCGGTGCTGTGCCAGGCCCACGGCTTGACCCCGGAGCAGGTGGTGG
CCATCGCCAGCCACGATGGCGGCAAGCAGGCGCTGGAGACGGTCCAGCGG
CTGTTGCCGGTGCTGTGCCAGGCCCACGGCTTGACCCCCCAGCAGGTGGT
GGCCATCGCCAGCAATGGCGGTGGCAAGCAGGCGCTGGAGACGGTCCAGC
GGCTGTTGCCGGTGCTGTGCCAGGCCCACGGCTTGACCCCCCAGCAGGTG
GTGGCCATCGCCAGCAATGGCGGTGGCAAGCAGGCGCTGGAGACGGTCCA
GCGGCTGTTGCCGGTGCTGTGCCAGGCCCACGGCTTGACCCCCCAGCAGG
TGGTGGCCATCGCCAGCAATGGCGGTGGCAAGCAGGCGCTGGAGACGGTC
CAGCGGCTGTTGCCGGTGCTGTGCCAGGCCCACGGCTTGACCCCGGAGCA
GGTGGTGGCCATCGCCAGCCACGATGGCGGCAAGCAGGCGCTGGAGACGG
TCCAGCGGCTGTTGCCGGTGCTGTGCCAGGCCCACGGCTTGACCCCCCAG
CAGGTGGTGGCCATCGCCAGCAATGGCGGTGGCAAGCAGGCGCTGGAGAC
GGTCCAGCGGCTGTTGCCGGTGCTGTGCCAGGCCCACGGCTTGACCCCCC
AGCAGGTGGTGGCCATCGCCAGCAATGGCGGTGGCAAGCAGGCGCTGGAG
ACGGTCCAGCGGCTGTTGCCGGTGCTGTGCCAGGCCCACGGCTTGACCCC
CCAGCAGGTGGTGGCCATCGCCAGCAATAATGGTGGCAAGCAGGCGCTGG
AGACGGTCCAGCGGCTGTTGCCGGTGCTGTGCCAGGCCCACGGCTTGACC
CCTCAGCAGGTGGTGGCCATCGCCAGCAATGGCGGCGGCAGGCCGGCGCT
GGAGAGCATTGTTGCCCAGTTATCTCGCCCTGATCCGGCGTTGGCCGCGT
TGACCAACGACCACCTCGTCGCCTTGGCCTGCCTCGGCGGGCGTCCTGCG
CTGGATGCAGTGAAAAAGGGATTGGGGGATCCTATCAGCCGTTCCCAGCT
GGTGAAGTCCGAGCTGGAGGAGAAGAAATCCGAGTTGAGGCACAAGCTGA
AGTACGTGCCCCACGAGTACATCGAGCTGATCGAGATCGCCCGGAACAGC
ACCCAGGACCGTATCCTGGAGATGAAGGTGATGGAGTTCTTCATGAAGGT
GTACGGCTACAGGGGCAAGCACCTGGGCGGCTCCAGGAAGCCCGACGGCG
CCATCTACACCGTGGGCTCCCCCATCGACTACGGCGTGATCGTGGACACC
AAGGCCTACTCCGGCGGCTACAACCTGCCCATCGGCCAGGCCGACGAAAT
GCAGAGGTACGTGGAGGAGAACCAGACCAGGAACAAGCACATCAACCCCA
ACGAGTGGTGGAAGGTGTACCCCTCCAGCGTGACCGAGTTCAAGTTCCTG
TTCGTGTCCGGCCACTTCAAGGGCAACTACAAGGCCCAGCTGACCAGGCT
GAACCACATCACCAACTGCAACGGCGCCGTGCTGTCCGTGGAGGAGCTCC
TGATCGGCGGCGAGATGATCAAGGCCGGCACCCTGACCCTGGAGGAGGTG
AGGAGGAAGTTCAACAACGGCGAGATCAACTTCGCGGCCGACTGATAACT
CGAGAAGGGCGCGATCGTTCAAACATTTGGCAATAAAGTTTCTTAAGATT
GAATCCTGTTGCCGGTCTTGCGATGATTATCATATAATTTCTGTTGAATT
ACGTTAAGCATGTAATAATTAACATGTAATGCATGACGTTATTTATGAGA
TGGGTTTTTATGATTAGAGTCCCGCAATTATACATTTAATACGCGATAGA
AAACAAAATATAGCGCGCAAACTAGGATAAATTATCGCGCGCGGTGTCAT
CTATGTTACTAGATCGGGAATTCGTAATCATGGTCATAGC
(full map of pCLS14)
SEQ ID NO: 32
TGATAATCTCATGACCAAAATCCCTTAACGTGAGTTTTCGTTCCACTGAG
CGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTG
GCTTCAGCAGAGCGCAGATACCAAATACTGTCCTTCTAGTGTAGCCGTAG
TTAGGCCACCACTTCAAGAACTCTGTAGCACCGCCTACATACCTCGCTCT
GCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGC
TGAACGGGGGGTTCGTGCACACAGCCCAGCTTGGAGCGAACGACCTACAC
CGAACTGAGATACCTACAGCGTGAGCATTGAGAAAGCGCCACGCTTCCCG
AAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGG
TTCCTGGCCTTTTGCTGGCCTTTTGCTCACATGTTCTTTCCTGCGTTATC
CCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCA
TTAATGCAGGTTAACCTGGCTTATCGAAATTAATACGACTCACTATAGGG
AGCCCGGCAGATCTGATCTCTTGAACTTTCCAAGAGTTGAAGAAAATCAC
AGAAAGCCTTAGCACAGAGAAGAGAGATTGAAGAAGTCGACGGCCATCGC
CAGCAATAATGGTGGCAAGCAGGCGCTGGAGACGGTCCAGCGGCTGTTGC
CGGTGCTGTGCCAGGCCCACGGCTTGACCCCGGAGCAGGTGGTGGCCATC
GCCAGCCACGATGGCGGCAAGCAGGCGCTGGAGACGGTCCAGCGGCTGTT
GCCGGTGCTGTGCCAGGCCCACGGCTTGACCCCCCAGCAGGTGGTGGCCA
TCGCCAGCAATAATGGTGGCAAGCAGGCGCTGGAGACGGTCCAGCGGCTG
TTGCCGGTGCTGTGCCAGGCCCACGGCTTGACCCCGGAGCAGGTGGTGGC
CATCGCCAGCAATATTGGTGGCAAGCAGGCGCTGGAGACGGTGCAGGCGC
TGTTGCCGGTGCTGTGCCAGGCCCACGGCTTGACCCCCCAGCAGGTGGTG
GCCATCGCCAGCAATAATGGTGGCAAGCAGGCGCTGGAGACGGTCCAGCG
GCTGTTGCCGGTGCTGTGCCAGGCCCACGGCTTGACCCCGGAGCAGGTGG
TGGCCATCGCCAGCAATATTGGTGGCAAGCAGGCGCTGGAGACGGTGCAG
GCGCTGTTGCCGGTGCTGTGCCAGGCCCACGGCTTGACCCCGGAGCAGGT
GGTGGCCATCGCCAGCCACGATGGCGGCAAGCAGGCGCTGGAGACGGTCC
AGCGGCTGTTGCCGGTGCTGTGCCAGGCCCACGGCTTGACCCCGGAGCAG
GTGGTGGCCATCGCCAGCAATATTGGTGGCAAGCAGGCGCTGGAGACGGT
GCAGGCGCTGTTGCCGGTGCTGTGCCAGGCCCACGGCTTGACCCCGGAGC
AGGTGGTGGCCATCGCCAGCCACGATGGCGGCAAGCAGGCGCTGGAGACG
GTCCAGCGGCTGTTGCCGGTGCTGTGCCAGGCCCACGGCTTGACCCCGGA
GCAGGTGGTGGCCATCGCCAGCCACGATGGCGGCAAGCAGGCGCTGGAGA
CGGTCCAGCGGCTGTTGCCGGTGCTGTGCCAGGCCCACGGCTTGACCCCC
CAGCAGGTGGTGGCCATCGCCAGCAATAATGGTGGCAAGCAGGCGCTGGA
GACGGTCCAGCGGCTGTTGCCGGTGCTGTGCCAGGCCCACGGCTTGACCC
CGGAGCAGGTGGTGGCCATCGCCAGCCACGATGGCGGCAAGCAGGCGCTG
GAGACGGTCCAGCGGCTGTTGCCGGTGCTGTGCCAGGCCCACGGCTTGAC
CCCGGAGCAGGTGGTGGCCATCGCCAGCCACGATGGCGGCAAGCAGGCGC
TGGAGACGGTCCAGCGGCTGTTGCCGGTGCTGTGCCAGGCCCACGGCTTG
ACCCCGGAGCAGGTGGTGGCCATCGCCAGCCACGATGGCGGCAAGCAGGC
GCTGGAGACGGTCCAGCGGCTGTTGCCGGTGCTGTGCCAGGCCCACGGCT
TGACCCCCCAGCAGGTGGTGGCCATCGCCAGCAATGGCGGTGGCAAGCAG
GCGCTGGAGACGGTCCAGCGGCTGTTGCCGGTGCTGTGCCAGGCCCACGG
CTTGACCCCTCAGCAGGTGGTGGCCATCGCCAGCAATGGCGGCGGCAGGC
CGGCGCTGGAGAGCATTGTTGCCCAGTTATCTCGCCCTGATCCGGCGTTG
GCCGCGTTGACCAACGACCACCTCGTCGCCTTGGCCTGCCTCGGCGGGCG
TCCTGCGCTGGATGCAGTGAAAAAGGGATTGGGGGATCCTATCAGCCGTT
CCCAGCTGGTGAAGTCCGAGCTGGAGGAGAAGAAATCCGAGTTGAGGCAC
AAGCTGAAGTACGTGCCCCACGAGTACATCGAGCTGATCGAGATCGCCCG
GAACAGCACCCAGGACCGTATCCTGGAGATGAAGGTGATGGAGTTCTTCA
TGAAGGTGTACGGCTACAGGGGCAAGCACCTGGGCGGCTCCAGGAAGCCC
GACGGCGCCATCTACACCGTGGGCTCCCCCATCGACTACGGCGTGATCGT
GGACACCAAGGCCTACTCCGGCGGCTACAACCTGCCCATCGGCCAGGCCG
ACGAAATGCAGAGGTACGTGGAGGAGAACCAGACCAGGAACAAGCACATC
AACCCCAACGAGTGGTGGAAGGTGTACCCCTCCAGCGTGACCGAGTTCAA
GTTCCTGTTCGTGTCCGGCCACTTCAAGGGCAACTACAAGGCCCAGCTGA
CCAGGCTGAACCACATCACCAACTGCAACGGCGCCGTGCTGTCCGTGGAG
GAGCTCCTGATCGGCGGCGAGATGATCAAGGCCGGCACCCTGACCCTGGA
GGAGGTGAGGAGGAAGTTCAACAACGGCGAGATCAACTTCGCGGCCGACT
GATAACCATGGAGAGGATATATATGTACATATGCAAAGGGATATCAAGAC
CATCTGTAATCTTTTGAAGTTTTGTGAAGCTATAGAAGCCAAGCAAGAAT
TCTACCAGATTACTTCCCAAATAAGTGGTGTGAATGTAAATTAATAAGAG
CTACAGAAACATTGATTGGCTCAGTGTATGTGTTGTATTCATATTCGTTG
TTTTATTTTATACGGTTGAGAATTGAATAATGTTGTTGCATCAAATCACT
ATGAAGGACATTTACAGTCAGCTGCTCGATCGAGGCGGCCAACAACAAAA
AAAAAAAAAAAAAAAAAAAAAAAAAAAAAAAAAAAAAAAAAAAAAAAAAA
AAAAAAAAAAGAAAAGCCAATTGGGATCNNAGTTCTATAGTGTCACCTAA
ATCGTATGTGTATGATACATAAGGTTATGTATTAATTGTAGCCGCGTTCT
AACGACAATATGTCCATATGGTGCACTCTCAGTACAATCTGCTCTGATGC
CGCATAGTTAAGCCAGCCCCGACACCCGCCAACACCCGCTGACGCGCCCT
GACGGGCTTGTCTGCTCCCGGCATCCGCTTACAGACAAGCTGTGACCGTC
TCCGGGAGCTGCATGTGTCAGAGGTTTTCACCGTCATCACCGAAACGCGC
GAGACGAAAGGGCCTCGTGATACGCCTATTTTTATAGGTTAATGTCATGA
TAATAATGGTTTCTTAGACGTCAGGTGGCACTTTTCGGGGAAATGTGCGC
GGAACCCCTATTTGTTTATTTTTCTAAATACATTCAAATATGTATCCGCT
CATGAGACAATAACCCTGATAAATGCTTCAATAATATTGAAAAAGGAAGA
GTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGA
TGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATG
ATGAGCACTTTTAAAGTTCTGCTATGTGGCGCGGTATTATCCCGTATTGA
CGCCGGGCAAGAGCAACTCGGTCGCCGCATACACTATTCTCAGAATGACT
TGGTTGAGTACTCACCAGTCACAGAAAAGCATCTTACGGATGGCATGACA
GTAAGAGAATTATGCAGTGCTGCCATAACCATGAGTGATAACACTGCGGC
CAACTTACTTCTGACAACGATCGGAGGACCGAAGGAGCTAACCGCTTTTT
TGCACAACATGGGGGATCATGTAACTCGCCTTGATCGTTGGGAACCGGAG
CTGAATGAAGCCATACCAAACGACGAGCGTGACACCACGATGCCTGTAGC
AATGGCAACAACGTTGCGCAAACTATTAACTGGCGAACTACTTACTCTAG
CTTCCCGGCAACAATTAATAGACTGGATGGAGGCGGATAAAGTTGCAGGA
CCACTTCTGCGCTCGGCCCTTCCGGCTGGCTGGTTTATTGCTGATAAATC
TGGAGCCGGTGAGCGTGGATCTCGCGGTATCATTGCAGCACTGGGGCCAG
ATGGTAAGCCCTCCCGTATCGTAGTTATCTACACGACGGGGAGTCAGGCA
ACTATGGATGAACGAAATAGACAGATCGCTGAGATAGGTGCCTCACTGAT
TAAGCATTGGTAACTGTCAGACCAAGTTTACTCATATATACTTTAGATTG
ATTTAAAACTTCATTTTTAATTTAAAAGGATCTAGGTGAAGATCCTTTT
(expression cassette from pCLS14)
SEQ ID NO: 33
TAATACGACTCACTATAGGGAGCCCGGCAGATCTGATCTCTTGAACTTTC
CAAGAGTTGAAGAAAATCACAGAAAGCCTTAGCACAGAGAAGAGAGATTG
AAGAAGTCGACGGCCATCGCCAGCAATAATGGTGGCAAGCAGGCGCTGGA
GACGGTCCAGCGGCTGTTGCCGGTGCTGTGCCAGGCCCACGGCTTGACCC
CGGAGCAGGTGGTGGCCATCGCCAGCCACGATGGCGGCAAGCAGGCGCTG
GAGACGGTCCAGCGGCTGTTGCCGGTGCTGTGCCAGGCCCACGGCTTGAC
CCCCCAGCAGGTGGTGGCCATCGCCAGCAATAATGGTGGCAAGCAGGCGC
TGGAGACGGTCCAGCGGCTGTTGCCGGTGCTGTGCCAGGCCCACGGCTTG
ACCCCGGAGCAGGTGGTGGCCATCGCCAGCAATATTGGTGGCAAGCAGGC
GCTGGAGACGGTGCAGGCGCTGTTGCCGGTGCTGTGCCAGGCCCACGGCT
TGACCCCCCAGCAGGTGGTGGCCATCGCCAGCAATAATGGTGGCAAGCAG
GCGCTGGAGACGGTCCAGCGGCTGTTGCCGGTGCTGTGCCAGGCCCACGG
CTTGACCCCGGAGCAGGTGGTGGCCATCGCCAGCAATATTGGTGGCAAGC
AGGCGCTGGAGACGGTGCAGGCGCTGTTGCCGGTGCTGTGCCAGGCCCAC
GGCTTGACCCCGGAGCAGGTGGTGGCCATCGCCAGCCACGATGGCGGCAA
GCAGGCGCTGGAGACGGTCCAGCGGCTGTTGCCGGTGCTGTGCCAGGCCC
ACGGCTTGACCCCGGAGCAGGTGGTGGCCATCGCCAGCAATATTGGTGGC
AAGCAGGCGCTGGAGACGGTGCAGGCGCTGTTGCCGGTGCTGTGCCAGGC
CCACGGCTTGACCCCGGAGCAGGTGGTGGCCATCGCCAGCCACGATGGCG
GCAAGCAGGCGCTGGAGACGGTCCAGCGGCTGTTGCCGGTGCTGTGCCAG
GCCCACGGCTTGACCCCGGAGCAGGTGGTGGCCATCGCCAGCCACGATGG
CGGCAAGCAGGCGCTGGAGACGGTCCAGCGGCTGTTGCCGGTGCTGTGCC
AGGCCCACGGCTTGACCCCCCAGCAGGTGGTGGCCATCGCCAGCAATAAT
GGTGGCAAGCAGGCGCTGGAGACGGTCCAGCGGCTGTTGCCGGTGCTGTG
CCAGGCCCACGGCTTGACCCCGGAGCAGGTGGTGGCCATCGCCAGCCACG
ATGGCGGCAAGCAGGCGCTGGAGACGGTCCAGCGGCTGTTGCCGGTGCTG
TGCCAGGCCCACGGCTTGACCCCGGAGCAGGTGGTGGCCATCGCCAGCCA
CGATGGCGGCAAGCAGGCGCTGGAGACGGTCCAGCGGCTGTTGCCGGTGC
TGTGCCAGGCCCACGGCTTGACCCCGGAGCAGGTGGTGGCCATCGCCAGC
CACGATGGCGGCAAGCAGGCGCTGGAGACGGTCCAGCGGCTGTTGCCGGT
GCTGTGCCAGGCCCACGGCTTGACCCCCCAGCAGGTGGTGGCCATCGCCA
GCAATGGCGGTGGCAAGCAGGCGCTGGAGACGGTCCAGCGGCTGTTGCCG
GTGCTGTGCCAGGCCCACGGCTTGACCCCTCAGCAGGTGGTGGCCATCGC
CAGCAATGGCGGCGGCAGGCCGGCGCTGGAGAGCATTGTTGCCCAGTTAT
CTCGCCCTGATCCGGCGTTGGCCGCGTTGACCAACGACCACCTCGTCGCC
TTGGCCTGCCTCGGCGGGCGTCCTGCGCTGGATGCAGTGAAAAAGGGATT
GGGGGATCCTATCAGCCGTTCCCAGCTGGTGAAGTCCGAGCTGGAGGAGA
AGAAATCCGAGTTGAGGCACAAGCTGAAGTACGTGCCCCACGAGTACATC
GAGCTGATCGAGATCGCCCGGAACAGCACCCAGGACCGTATCCTGGAGAT
GAAGGTGATGGAGTTCTTCATGAAGGTGTACGGCTACAGGGGCAAGCACC
TGGGCGGCTCCAGGAAGCCCGACGGCGCCATCTACACCGTGGGCTCCCCC
ATCGACTACGGCGTGATCGTGGACACCAAGGCCTACTCCGGCGGCTACAA
CCTGCCCATCGGCCAGGCCGACGAAATGCAGAGGTACGTGGAGGAGAACC
AGACCAGGAACAAGCACATCAACCCCAACGAGTGGTGGAAGGTGTACCCC
TCCAGCGTGACCGAGTTCAAGTTCCTGTTCGTGTCCGGCCACTTCAAGGG
CAACTACAAGGCCCAGCTGACCAGGCTGAACCACATCACCAACTGCAACG
GCGCCGTGCTGTCCGTGGAGGAGCTCCTGATCGGCGGCGAGATGATCAAG
GCCGGCACCCTGACCCTGGAGGAGGTGAGGAGGAAGTTCAACAACGGCGA
GATCAACTTCGCGGCCGACTGATAACCATGGAGAGGATATATATGTACAT
ATGCAAAGGGATATCAAGACCATCTGTAATCTTTTGAAGTTTTGTGAAGC
TATAGAAGCCAAGCAAGAATTCTACCAGATTACTTCCCAAATAAGTGGTG
TGAATGTAAATTAATAAGAGCTACAGAAACATTGATTGGCTCAGTGTATG
TGTTGTATTCATATTCGTTGTTTTATTTTATACGGTTGAGAATTGAATAA
TGTTGTTGCATCAAATCACTATGAAGGACATTTACAGTCAGCTGCTCGAT
CGAGGCGGCCAACAACAAAAAAAAAAAAAAAAAAAAAAAAAAAAAAAAAA
AAAAAAAAAAAAAAAAAAAAAAAAAAAAAAGAAAAGCCAATTGGGATCNN
AGTTCTATAGTGTCACCTAAATCGTATGTGTATGATACATAAGGTTATGT
ATTAATTGTAGCCGCGTTCTAACGACAATATGTCCATATGGTGCACTCTC
AGTACAATCTGCTCTGATGCCGCATAGTTAAGCCAGCCCCGACACCCGCC
AACACCCGCTGACGCGCCCTGACGGGCTTGTCTGCTCCCGGCATCCGCTT
A
(full map of pCLS15)
SEQ ID NO: 34
TGATAATCTCATGACCAAAATCCCTTAACGTGAGTTTTCGTTCCACTGAG
CGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGT
GGTTTGTTTGCCGGATCAAGAGCTACCAACTCTTTTTCCGAAGGTAACTG
GCTTCAGCAGAGCGCAGATACCAAATACTGTCCTTCTAGTGTAGCCGTAG
TTAGGCCACCACTTCAAGAACTCTGTAGCACCGCCTACATACCTCGCTCT
GCTAATCCTGTTACCAGTGGCTGCTGCCAGTGGCGATAAGTCGTGTCTTA
CCGGGTTGGACTCAAGACGATAGTTACCGGATAAGGCGCAGCGGTCGGGC
TGAACGGGGGGTTCGTGCACACAGCCCAGCTTGGAGCGAACGACCTACAC
CGAACTGAGATACCTACAGCGTGAGCATTGAGAAAGCGCCACGCTTCCCG
AAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGG
TTCCTGGCCTTTTGCTGGCCTTTTGCTCACATGTTCTTTCCTGCGTTATC
CCCTGATTCTGTGGATAACCGTATTACCGCCTTTGAGTGAGCTGATACCG
CTCGCCGCAGCCGAACGACCGAGCGCAGCGAGTCAGTGAGCGAGGAAGCG
GAAGAGCGCCCAATACGCAAACCGCCTCTCCCCGCGCGTTGGCCGATTCA
TTAATGCAGGTTAACCTGGCTTATCGAAATTAATACGACTCACTATAGGG
AGCCCGGCAGATCTGATCTCTTGAACTTTCCAAGAGTTGAAGAAAATCAC
AGAAAGCCTTAGCACAGAGAAGAGAGATTGAAGAAGTCGACATGGGCGAT
CCTAAAAAGAAACGTAAGGTCATCGATAAGGAGACTGCCGCTGCCAAGTT
CGAGAGACAGCACATGGACAGCATGGTGTCTAAGGGCGAAGAGCTGATTA
AGGAGAACATGCACATGAAGCTGTACATGGAGGGCACCGTGAACAACCAC
CACTTCAAGTGCACATCCGAGGGCGAAGGCAAGCCCTACGAGGGCACCCA
GACCATGAGAATCAAGGTGGTCGAGGGCGGCCCTCTCCCCTTCGCCTTCG
ACATCCTGGCTACCAGCTTCATGTACGGCAGCAGAACCTTCATCAACCAC
ACCCAGGGCATCCCCGACTTCTTTAAGCAGTCCTTCCCTGAGGGCTTCAC
ATGGGAGAGAGTCACCACATACGAAGACGGGGGCGTGCTGACCGCTACCC
AGGACACCAGCCTCCAGGACGGCTGCCTCATCTACAACGTCAAGATCAGA
GGGGTGAACTTCCCATCCAACGGCCCTGTGATGCAGAAGAAAACACTCGG
CTGGGAGGCCAACACCGAGATGCTGTACCCCGCTGACGGCGGCCTGGAAG
GCAGAAGCGACATGGCCCTGAAGCTCGTGGGCGGGGGCCACCTGATCTGC
AACTTCAAGACCACATACAGATCCAAGAAACCCGCTAAGAACCTCAAGAT
GCCCGGCGTCTACTATGTGGACCACAGACTGGAAAGAATCAAGGAGGCCG
ACAAAGAGACGTACGTCGAGCAGCACGAGGTGGCTGTGGCCAGATACTGC
GACCTCCCTAGCAAACTGGGGCACAAACTTAATGGAGGGGGCGGTAGCGG
CGGTGGCGGGAGCATCGATATCGCCGATCTACGCACGCTCGGCTACAGCC
AGCAGCAACAGGAGAAGATCAAACCGAAGGTTCGTTCGACAGTGGCGCAG
CACCACGAGGCACTGGTCGGCCACGGGTTTACACACGCGCACATCGTTGC
GTTAAGCCAACACCCGGCAGCGTTAGGGACCGTCGCTGTCAAGTATCAGG
ACATGATCGCAGCGTTGCCAGAGGCGACACACGAAGCGATCGTTGGCGTC
GGCAAACAGTGGTCCGGCGCACGCGCTCTGGAGGCCTTGCTCACGGTGGC
GGGAGAGTTGAGAGGTCCACCGTTACAGTTGGACACAGGCCAACTTCTCA
AGATTGCAAAACGTGGCGGCGTGACCGCAGTGGAGGCAGTGCATGCATGG
CGCAATGCACTGACGGGTGCCCCGCTCAACTTGACCCCCCAGCAGGTGGT
GGCCATCGCCAGCAATAATGGTGGCAAGCAGGCGCTGGAGACGGTCCAGC
GGCTGTTGCCGGTGCTGTGCCAGGCCCACGGCTTGACCCCCCAGCAGGTG
GTGGCCATCGCCAGCAATAATGGTGGCAAGCAGGCGCTGGAGACGGTCCA
GCGGCTGTTGCCGGTGCTGTGCCAGGCCCACGGCTTGACCCCCCAGCAGG
TGGTGGCCATCGCCAGCAATAATGGTGGCAAGCAGGCGCTGGAGACGGTC
CAGCGGCTGTTGCCGGTGCTGTGCCAGGCCCACGGCTTGACCCCGGAGCA
GGTGGTGGCCATCGCCAGCAATATTGGTGGCAAGCAGGCGCTGGAGACGG
TGCAGGCGCTGTTGCCGGTGCTGTGCCAGGCCCACGGCTTGACCCCCCAG
CAGGTGGTGGCCATCGCCAGCAATGGCGGTGGCAAGCAGGCGCTGGAGAC
GGTCCAGCGGCTGTTGCCGGTGCTGTGCCAGGCCCACGGCTTGACCCCCC
AGCAGGTGGTGGCCATCGCCAGCAATGGCGGTGGCAAGCAGGCGCTGGAG
ACGGTCCAGCGGCTGTTGCCGGTGCTGTGCCAGGCCCACGGCTTGACCCC
CCAGCAGGTGGTGGCCATCGCCAGCAATAATGGTGGCAAGCAGGCGCTGG
AGACGGTCCAGCGGCTGTTGCCGGTGCTGTGCCAGGCCCACGGCTTGACC
CCGGAGCAGGTGGTGGCCATCGCCAGCCACGATGGCGGCAAGCAGGCGCT
GGAGACGGTCCAGCGGCTGTTGCCGGTGCTGTGCCAGGCCCACGGCTTGA
CCCCCCAGCAGGTGGTGGCCATCGCCAGCAATGGCGGTGGCAAGCAGGCG
CTGGAGACGGTCCAGCGGCTGTTGCCGGTGCTGTGCCAGGCCCACGGCTT
GACCCCCCAGCAGGTGGTGGCCATCGCCAGCAATGGCGGTGGCAAGCAGG
CGCTGGAGACGGTCCAGCGGCTGTTGCCGGTGCTGTGCCAGGCCCACGGC
TTGACCCCCCAGCAGGTGGTGGCCATCGCCAGCAATGGCGGTGGCAAGCA
GGCGCTGGAGACGGTCCAGCGGCTGTTGCCGGTGCTGTGCCAGGCCCACG
GCTTGACCCCGGAGCAGGTGGTGGCCATCGCCAGCCACGATGGCGGCAAG
CAGGCGCTGGAGACGGTCCAGCGGCTGTTGCCGGTGCTGTGCCAGGCCCA
CGGCTTGACCCCCCAGCAGGTGGTGGCCATCGCCAGCAATGGCGGTGGCA
AGCAGGCGCTGGAGACGGTCCAGCGGCTGTTGCCGGTGCTGTGCCAGGCC
CACGGCTTGACCCCCCAGCAGGTGGTGGCCATCGCCAGCAATGGCGGTGG
CAAGCAGGCGCTGGAGACGGTCCAGCGGCTGTTGCCGGTGCTGTGCCAGG
CCCACGGCTTGACCCCCCAGCAGGTGGTGGCCATCGCCAGCAATAATGGT
GGCAAGCAGGCGCTGGAGACGGTCCAGCGGCTGTTGCCGGTGCTGTGCCA
GGCCCACGGCTTGACCCCTCAGCAGGTGGTGGCCATCGCCAGCAATGGCG
GCGGCAGGCCGGCGCTGGAGAGCATTGTTGCCCAGTTATCTCGCCCTGAT
CCGGCGTTGGCCGCGTTGACCAACGACCACCTCGTCGCCTTGGCCTGCCT
CGGCGGGCGTCCTGCGCTGGATGCAGTGAAAAAGGGATTGGGGGATCCTA
TCAGCCGTTCCCAGCTGGTGAAGTCCGAGCTGGAGGAGAAGAAATCCGAG
TTGAGGCACAAGCTGAAGTACGTGCCCCACGAGTACATCGAGCTGATCGA
GATCGCCCGGAACAGCACCCAGGACCGTATCCTGGAGATGAAGGTGATGG
AGTTCTTCATGAAGGTGTACGGCTACAGGGGCAAGCACCTGGGCGGCTCC
AGGAAGCCCGACGGCGCCATCTACACCGTGGGCTCCCCCATCGACTACGG
CGTGATCGTGGACACCAAGGCCTACTCCGGCGGCTACAACCTGCCCATCG
GCCAGGCCGACGAAATGCAGAGGTACGTGGAGGAGAACCAGACCAGGAAC
AAGCACATCAACCCCAACGAGTGGTGGAAGGTGTACCCCTCCAGCGTGAC
CGAGTTCAAGTTCCTGTTCGTGTCCGGCCACTTCAAGGGCAACTACAAGG
CCCAGCTGACCAGGCTGAACCACATCACCAACTGCAACGGCGCCGTGCTG
TCCGTGGAGGAGCTCCTGATCGGCGGCGAGATGATCAAGGCCGGCACCCT
GACCCTGGAGGAGGTGAGGAGGAAGTTCAACAACGGCGAGATCAACTTCG
CGGCCGACTGATAAAGAGGATATATATGTACATATGCAAAGGGATATCAA
GACCATCTGTAATCTTTTGAAGTTTTGTGAAGCTATAGAAGCCAAGCAAG
AATTCTACCAGATTACTTCCCAAATAAGTGGTGTGAATGTAAATTAATAA
GAGCTACGAAACATTGATTGGCTCAGTGTATGTGTTGTATTCATATTCGT
TGTTTTATTTTATACGGTTGAGAATTGAATAATGTTGTTGCATCAAATCA
CTATGAAGGACATTTACAGTCAGCTGCTCGATCGAGGCGGCCAACAACAA
AAAAAAAAAAAAAAAAAAAAAAAAAAAAAAAAAAAAAAAAAAGAAGAGCC
AATTGGGATCNNAGTTCTATAGTGTCACCTAAATCGTATGTGTATGATAC
ATAAGGTTATGTATTAATTGTAGCCGCGTTCTAACGACAATATGTCCATA
TGGTGCACTCTCAGTACAATCTGCTCTGATGCCGCATAGTTAAGCCAGCC
CCGACACCCGCCAACACCCGCTGACGCGCCCTGACGGGCTTGTCTGCTCC
CGGCATCCGCTTACAGACAAGCTGTGACCGTCTCCGGGAGCTGCATGTGT
CAGAGGTTTTCACCGTCATCACCGAAACGCGCGAGACGAAAGGGCCTCGT
GATACGCCTATTTTTATAGGTTAATGTCATGATAATAATGGTTTCTTAGA
CGTCAGGTGGCACTTTTCGGGGAAATGTGCGCGGAACCCCTATTTGTTTA
TTTTTCTAAATACATTCAAATATGTATCCGCTCATGAGACAATAACCCTG
ATAAATGCTTCAATAATATTGAAAAAGGAAGAGTATGAGTATTCAACATT
TCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGG
TGCACGAGTGGGTTACATCGAACTGGATCTCAACAGCGGTAAGATCCTTG
AGAGTTTTCGCCCCGAAGAACGTTTTCCAATGATGAGCACTTTTAAAGTT
CTGCTATGTGGCGCGGTATTATCCCGTATTGACGCCGGGCAAGAGCAACT
CGGTCGCCGCATACACTATTCTCAGAATGACTTGGTTGAGTACTCACCAG
TCACAGAAAAGCATCTTACGGATGGCATGACAGTAAGAGAATTATGCAGT
GCTGCCATAACCATGAGTGATAACACTGCGGCCAACTTACTTCTGACAAC
GATCGGAGGACCGAAGGAGCTAACCGCTTTTTTGCACAACATGGGGGATC
ATGTAACTCGCCTTGATCGTTGGGAACCGGAGCTGAATGAAGCCATACCA
AACGACGAGCGTGACACCACGATGCCTGTAGCAATGGCAACAACGTTGCG
CAAACTATTAACTGGCGAACTACTTACTCTAGCTTCCCGGCAACAATTAA
TAGACTGGATGGAGGCGGATAAAGTTGCAGGACCACTTCTGCGCTCGGCC
CTTCCGGCTGGCTGGTTTATTGCTGATAAATCTGGAGCCGGTGAGCGTGG
ATCTCGCGGTATCATTGCAGCACTGGGGCCAGATGGTAAGCCCTCCCGTA
TCGTAGTTATCTACACGACGGGGAGTCAGGCAACTATGGATGAACGAAAT
AGACAGATCGCTGAGATAGGTGCCTCACTGATTAAGCATTGGTAACTGTC
AGACCAAGTTTACTCATATATACTTTAGATTGATTTAAAACTTCATTTTT
AATTTAAAAGGATCTAGGTGAAGATCCTTTT
(expression cassette from pCLS15)
SEQ ID NO: 35
TAATACGACTCACTATAGGGAGCCCGGCAGATCTGATCTCTTGAACTTTC
CAAGAGTTGAAGAAAATCACAGAAAGCCTTAGCACAGAGAAGAGAGATTG
AAGAAGTCGACATGGGCGATCCTAAAAAGAAACGTAAGGTCATCGATAAG
GAGACTGCCGCTGCCAAGTTCGAGAGACAGCACATGGACAGCATGGTGTC
TAAGGGCGAAGAGCTGATTAAGGAGAACATGCACATGAAGCTGTACATGG
AGGGCACCGTGAACAACCACCACTTCAAGTGCACATCCGAGGGCGAAGGC
AAGCCCTACGAGGGCACCCAGACCATGAGAATCAAGGTGGTCGAGGGCGG
CCCTCTCCCCTTCGCCTTCGACATCCTGGCTACCAGCTTCATGTACGGCA
GCAGAACCTTCATCAACCACACCCAGGGCATCCCCGACTTCTTTAAGCAG
TCCTTCCCTGAGGGCTTCACATGGGAGAGAGTCACCACATACGAAGACGG
GGGCGTGCTGACCGCTACCCAGGACACCAGCCTCCAGGACGGCTGCCTCA
TCTACAACGTCAAGATCAGAGGGGTGAACTTCCCATCCAACGGCCCTGTG
ATGCAGAAGAAAACACTCGGCTGGGAGGCCAACACCGAGATGCTGTACCC
CGCTGACGGCGGCCTGGAAGGCAGAAGCGACATGGCCCTGAAGCTCGTGG
GCGGGGGCCACCTGATCTGCAACTTCAAGACCACATACAGATCCAAGAAA
CCCGCTAAGAACCTCAAGATGCCCGGCGTCTACTATGTGGACCACAGACT
GGAAAGAATCAAGGAGGCCGACAAAGAGACGTACGTCGAGCAGCACGAGG
TGGCTGTGGCCAGATACTGCGACCTCCCTAGCAAACTGGGGCACAAACTT
AATGGAGGGGGCGGTAGCGGCGGTGGCGGGAGCATCGATATCGCCGATCT
ACGCACGCTCGGCTACAGCCAGCAGCAACAGGAGAAGATCAAACCGAAGG
TTCGTTCGACAGTGGCGCAGCACCACGAGGCACTGGTCGGCCACGGGTTT
ACACACGCGCACATCGTTGCGTTAAGCCAACACCCGGCAGCGTTAGGGAC
CGTCGCTGTCAAGTATCAGGACATGATCGCAGCGTTGCCAGAGGCGACAC
ACGAAGCGATCGTTGGCGTCGGCAAACAGTGGTCCGGCGCACGCGCTCTG
GAGGCCTTGCTCACGGTGGCGGGAGAGTTGAGAGGTCCACCGTTACAGTT
GGACACAGGCCAACTTCTCAAGATTGCAAAACGTGGCGGCGTGACCGCAG
TGGAGGCAGTGCATGCATGGCGCAATGCACTGACGGGTGCCCCGCTCAAC
TTGACCCCCCAGCAGGTGGTGGCCATCGCCAGCAATAATGGTGGCAAGCA
GGCGCTGGAGACGGTCCAGCGGCTGTTGCCGGTGCTGTGCCAGGCCCACG
GCTTGACCCCCCAGCAGGTGGTGGCCATCGCCAGCAATAATGGTGGCAAG
CAGGCGCTGGAGACGGTCCAGCGGCTGTTGCCGGTGCTGTGCCAGGCCCA
CGGCTTGACCCCCCAGCAGGTGGTGGCCATCGCCAGCAATAATGGTGGCA
AGCAGGCGCTGGAGACGGTCCAGCGGCTGTTGCCGGTGCTGTGCCAGGCC
CACGGCTTGACCCCGGAGCAGGTGGTGGCCATCGCCAGCAATATTGGTGG
CAAGCAGGCGCTGGAGACGGTGCAGGCGCTGTTGCCGGTGCTGTGCCAGG
CCCACGGCTTGACCCCCCAGCAGGTGGTGGCCATCGCCAGCAATGGCGGT
GGCAAGCAGGCGCTGGAGACGGTCCAGCGGCTGTTGCCGGTGCTGTGCCA
GGCCCACGGCTTGACCCCCCAGCAGGTGGTGGCCATCGCCAGCAATGGCG
GTGGCAAGCAGGCGCTGGAGACGGTCCAGCGGCTGTTGCCGGTGCTGTGC
CAGGCCCACGGCTTGACCCCCCAGCAGGTGGTGGCCATCGCCAGCAATAA
TGGTGGCAAGCAGGCGCTGGAGACGGTCCAGCGGCTGTTGCCGGTGCTGT
GCCAGGCCCACGGCTTGACCCCGGAGCAGGTGGTGGCCATCGCCAGCCAC
GATGGCGGCAAGCAGGCGCTGGAGACGGTCCAGCGGCTGTTGCCGGTGCT
GTGCCAGGCCCACGGCTTGACCCCCCAGCAGGTGGTGGCCATCGCCAGCA
ATGGCGGTGGCAAGCAGGCGCTGGAGACGGTCCAGCGGCTGTTGCCGGTG
CTGTGCCAGGCCCACGGCTTGACCCCCCAGCAGGTGGTGGCCATCGCCAG
CAATGGCGGTGGCAAGCAGGCGCTGGAGACGGTCCAGCGGCTGTTGCCGG
TGCTGTGCCAGGCCCACGGCTTGACCCCCCAGCAGGTGGTGGCCATCGCC
AGCAATGGCGGTGGCAAGCAGGCGCTGGAGACGGTCCAGCGGCTGTTGCC
GGTGCTGTGCCAGGCCCACGGCTTGACCCCGGAGCAGGTGGTGGCCATCG
CCAGCCACGATGGCGGCAAGCAGGCGCTGGAGACGGTCCAGCGGCTGTTG
CCGGTGCTGTGCCAGGCCCACGGCTTGACCCCCCAGCAGGTGGTGGCCAT
CGCCAGCAATGGCGGTGGCAAGCAGGCGCTGGAGACGGTCCAGCGGCTGT
TGCCGGTGCTGTGCCAGGCCCACGGCTTGACCCCCCAGCAGGTGGTGGCC
ATCGCCAGCAATGGCGGTGGCAAGCAGGCGCTGGAGACGGTCCAGCGGCT
GTTGCCGGTGCTGTGCCAGGCCCACGGCTTGACCCCCCAGCAGGTGGTGG
CCATCGCCAGCAATAATGGTGGCAAGCAGGCGCTGGAGACGGTCCAGCGG
CTGTTGCCGGTGCTGTGCCAGGCCCACGGCTTGACCCCTCAGCAGGTGGT
GGCCATCGCCAGCAATGGCGGCGGCAGGCCGGCGCTGGAGAGCATTGTTG
CCCAGTTATCTCGCCCTGATCCGGCGTTGGCCGCGTTGACCAACGACCAC
CTCGTCGCCTTGGCCTGCCTCGGCGGGCGTCCTGCGCTGGATGCAGTGAA
AAAGGGATTGGGGGATCCTATCAGCCGTTCCCAGCTGGTGAAGTCCGAGC
TGGAGGAGAAGAAATCCGAGTTGAGGCACAAGCTGAAGTACGTGCCCCAC
GAGTACATCGAGCTGATCGAGATCGCCCGGAACAGCACCCAGGACCGTAT
CCTGGAGATGAAGGTGATGGAGTTCTTCATGAAGGTGTACGGCTACAGGG
GCAAGCACCTGGGCGGCTCCAGGAAGCCCGACGGCGCCATCTACACCGTG
GGCTCCCCCATCGACTACGGCGTGATCGTGGACACCAAGGCCTACTCCGG
CGGCTACAACCTGCCCATCGGCCAGGCCGACGAAATGCAGAGGTACGTGG
AGGAGAACCAGACCAGGAACAAGCACATCAACCCCAACGAGTGGTGGAAG
GTGTACCCCTCCAGCGTGACCGAGTTCAAGTTCCTGTTCGTGTCCGGCCA
CTTCAAGGGCAACTACAAGGCCCAGCTGACCAGGCTGAACCACATCACCA
ACTGCAACGGCGCCGTGCTGTCCGTGGAGGAGCTCCTGATCGGCGGCGAG
ATGATCAAGGCCGGCACCCTGACCCTGGAGGAGGTGAGGAGGAAGTTCAA
CAACGGCGAGATCAACTTCGCGGCCGACTGATAAAGAGGATATATATGTA
CATATGCAAAGGGATATCAAGACCATCTGTAATCTTTTGAAGTTTTGTGA
AGCTATAGAAGCCAAGCAAGAATTCTACCAGATTACTTCCCAAATAAGTG
GTGTGAATGTAAATTAATAAGAGCTACGAAACATTGATTGGCTCAGTGTA
TGTGTTGTATTCATATTCGTTGTTTTATTTTATACGGTTGAGAATTGAAT
AATGTTGTTGCATCAAATCACTATGAAGGACATTTACAGTCAGCTGCTCG
ATCGAGGCGGCCAACAACAAAAAAAAAAAAAAAAAAAAAAAAAAAAAAAA
AAAAAAAAAAAAGAAGA

1. A method, comprising:
contacting a population of plant cells with a messenger ribonucleic acid (mRNA) construct including a sequence encoding a rare-cutting endonuclease and a detectable label, wherein the rare-cutting endonuclease is configured to induce a mutation at a target genomic locus; and
screening the population of plant cells for the detectable label to identify target plant cells that are genetically transformed with the mRNA construct.

2. The method of claim 1, wherein contacting the population of plant cells includes delivering the mRNA construct into the population of plant cells derived using at least one of polyethylene glycol (PEG)-mediated transformation, electroporation, particle bombardment, and microinjection mediated protoplast transformation.

3. The method of claim 1, further including preparing the mRNA construct using in-vitro transcription, wherein the mRNA construct includes a transcription activator like effector nuclease (TALEN) mRNA including the sequence encoding the rare-cutting endonuclease and the detectable label.

4. The method of claim 1, wherein the rare-cutting endonuclease is conjugated to the detectable label or is a fusion protein including the rare-cutting endonuclease and the detectable label.

5. The method of one of claim 1, wherein screening the population of plant cells for the detectable label includes isolating the target plant cells that have the detectable label from a remainder of the population of plant cells.

6. The method of claim 5, wherein the detectable label includes a first detectable label and a second detectable label and wherein the rare-cutting endonuclease includes a first half-transcription activator like effector nuclease (TALEN) that is labeled with the first detectable label and a second half-TALEN that is labeled with the second detectable label, and wherein isolating the target plant cells from the remainder includes isolating the target plant cells that have the first detectable label and the second detectable label.

7. The method of claim 5, wherein isolating the target plant cells includes using fluorescence activated cell sorting (FACS) with a nozzle having a diameter of at least 100 um and up to 200 um.

8. The method of claim 1, wherein the plant cells are plant protoplasts and the method further includes:
culturing the target plant cells that are transformed with the mRNA construct; and
regenerating plants from the cultured target plant cells, wherein the regenerated plants express the mRNA construct.

9. A non-naturally occurring plant, generated by a genomic editing technique, wherein the genomic editing technique comprises:
contacting a population of plant cells with a messenger ribonucleic acid (mRNA) construct that includes a sequence encoding a rare-cutting endonuclease and a detectable label, wherein the rare-cutting endonuclease is configured to induce a mutation at a target genomic locus;
screening the population of plant cells for the detectable label to identify target plant cells that are transformed with the mRNA construct; and
regenerating a non-naturally occurring plant from the target plant cells.

10. The non-naturally occurring plant of claim 9, wherein the mRNA construct comprises an mRNA coding sequence including:
a rare-cutting endonuclease sequence encoding the rare-cutting endonuclease; and
a detectable label sequence encoding the detectable label.

11. A messenger ribonucleic acid (mRNA) construct, comprising:
an mRNA coding sequence including:
a rare-cutting endonuclease sequence; and
a detectable label sequence; and
a promoter sequence upstream from the mRNA coding sequence.

12. The mRNA construct of claim 11, further including a first untranslated region (UTR) upstream from the mRNA coding sequence and downstream from the promoter sequence.

13. The mRNA construct of claim 12, further include a second UTR that is downstream from the mRNA coding sequence.

14. The mRNA construct of claim 11, wherein the rare-cutting endonuclease sequence includes a sequence encoding a transcription activator like effector nuclease (TALEN).

15. The mRNA construct of claim 14, wherein the detectable label sequence encodes a first detectable label and a second detectable label, and the rare-cutting endonuclease encodes a first half-TALEN that is labeled with the first detectable label and a second half-TALEN that is labeled with the second detectable label.

16. The mRNA construct of claim 15, wherein the first detectable label and the second detectable label are different.

17. The mRNA construct of claim 15, wherein:
the first half-TALEN includes a first binding domain and a first endonuclease domain that forms a first fusion protein with the first detectable label; and
the second half-TALEN includes a second binding domain and a second endonuclease domain that forms a second fusion protein a second detectable label.

18. The mRNA construct of claim 15, wherein the first detectable label and the second detectable label each include a fluorescent protein.

19. The mRNA construct of claim 11, wherein the mRNA construct encodes the rare-cutting endonuclease sequence and the detectable label sequence separated by a flexible linker sequence.

20. The mRNA construct of claim 11, wherein the detectable label sequence includes a detectably labeled nucleotide.
